(19) United States
(12) Patent Application Publication
(10) Pub. No.: US 2025/0049928 A1
(43) Pub. Date: Feb. 13, 2025

(54) ANESTHETIC PRODRUGS AND DERIVATIVES THEREOF FOR PROLONGED SENSORY SELECTIVE NERVE BLOCKAGE

(71) Applicant: The Board of Trustees of The University of Alabama, Tuscaloosa, AL (US)

(72) Inventor: Chao Zhao, Tuscaloosa, AL (US)

(21) Appl. No.: 18/675,534

(22) Filed: May 28, 2024

Related U.S. Application Data
(60) Provisional application No. 63/528,732, filed on Jul. 25, 2023.

(51) Int. Cl. A61K 47/55; A61K 47/54; A61P 23/00

(52) U.S. Cl. CPC A61K 47/55; A61K 47/549; A61P 23/00

(57) ABSTRACT

Prodrugs and derivatives of anesthetics (e.g., capsaicin, bupivacaine) and methods of using the same.

### CROSS REFERENCE TO RELATED APPLICATIONS

This application claims the benefit of priority to U.S. Provisional Application No. 63/528,732, filed Jul. 25, 2023, which is incorporated by reference herein in its entirety.

### GOVERNMENT SUPPORT CLAUSE

This invention was made with government support under Grant No. R61 NS123196 awarded by the National Institutes of Health. The government has certain rights in the invention.

### BACKGROUND

Injection of conventional amino-amide and amino-ester local anesthetics around nerves can effectively inhibit peripheral nerve signal transduction and relieve pain. Duration of local anesthetic nerve blockade can be extended by perineural catheters, addition of adjuvant drug, or drug formulation into sustained release systems, such as liposomes or nanoparticles. However, these approaches produce unwanted motor nerve blockade of approximately the same duration as the intended sensory blockade, rendering them clinically unsuitable for post-operative or chronic pain control involving weightbearing extremities. As a consequence, patients have to deal with varying degrees of muscle weakness for adequate pain relief. This is particularly problematic for postoperative pain management that typically lasts for 5-7 days and long-term chronic pain management for as long as 12 weeks.

Capsaicin is a highly selective non-opioid agonist for the transient receptor potential vanilloid-receptor type 1 (TRPV1) channel. It can bind to residue Tyr511 to open the TRPV1 receptor that facilitates electrical impulse transmission to the central nervous system. Intradermal administration of capsaicin results in a nearly complete degeneration of epidermal nerve fibers and the subepidermal neural plexus suggesting that sensory dysfunction after capsaicin application to the skin results from rapid degeneration of intracutaneous nerve fibers. Since TRPV1 is expressed primarily on the central and peripheral terminals of nociceptive neurons in axons and the dorsal root ganglion cells (DRGs) [12], capsaicin selectively acts on nociceptive signaling without motor nerve blockade or disruption of proprioception and touch sensation. As a consequence, capsaicin is a potential sensory-selective blocking agent for nociceptive signals. The use of capsaicin to inhibit nociceptive signaling without impairing motor function has recently been reported [13]. Perineural injection of capsaicin with QX-314, a quaternary lidocaine derivative with obligate positive charges, produces a nociceptive-selective axon blockade lasting 2 hours in rats. However, 2 hours is relatively short, making this formulation clinically inadequate for post-operative pain and chronic pain syndromes lasting days to weeks.

Applying capsaicin for prolonged duration nociceptive-selective axon blockade poses two major challenges: i) Insufficient drug permeation due to peripheral nerve barriers (PNBs). In order to act on nociceptive sensory neurons, the perineurally injected capsaicin must first cross the tight junction forming restrictive perineurium composed of multiple concentric layers of specialized epithelioid myofibroblasts. It is well established that a very small percentage (<1%) of local anesthetics injected subcutaneously penetrate the perineurium to subsequently modulate axonal signal transduction [14]. These drugs are significantly adsorbed into adjacent tissues and/or taken up into the systemic circulation. ii) Dose limitation due to the potential local and systemic drug toxicity. Local toxic effects include irritation, redness, and burning sensation at the injection site, while systemic adverse effects can lead to dizziness, nausea, vomiting, hypotension, tachycardia, seizures, and respiratory depression [15]. Moreover, capsaicin toxicity can cause myotoxicity and neurotoxicity, leading to muscle weakness, poor stamina, and lack of muscle control, particularly with prolonged use or high doses [16].

Thus, there is a need for compounds, compositions, and methods effective for administering long duration sensory blockade without muscle weakness or paralysis (e.g., sensory-selective nerve blockade). These needs and others are at least partially satisfied by the present invention.

### SUMMARY

Disclosed herein, in one aspect, are sugar-capsaicin prodrugs, in which capsaicin or a derivative thereof act only on nociceptive sensory neurons and not on motor neurons, producing a nociceptive-selective axon blockade (i.e. a specific type of sensory-selective nerve blockade that blocks nociceptive signaling without motor nerve blockade and disruption of proprioception and touch sensation). The prodrug's sugar moiety can enhance permeability across the tight junction forming restrictive peripheral nerve barriers (PNBs) via carrier-mediated transport by facilitative glucose transporter 1 (GLUT1). The sugar-capsaicin prodrugs can enhance capsaicin's bioavailability to nociceptive sensory neurons, leading to reduced capsaicin absorbed by neighboring tissues or taken up by systemic circulation. This process can enhance analgesic drug effect with reduced adverse effects. Furthermore, the prodrug can be gradually converted to active drug, capsaicin, through linker hydrolysis in the endoneurium after crossing the PNBs, resulting in sustained drug release. The compounds disclosed herein can support safe administration of higher capsaicin doses and extend the duration of nociceptive-selective axon blockade.

The disclosed compounds can have a structure represented by Formula I:

Z-L-X (I)

or pharmaceutically acceptable salt thereof, wherein: Z is an anesthetic having a functionalizable o or N atom; L is an optional divalent linker; X is a monosaccharide, a disaccharide, or H; L, if present, is covalently bonded to X by —O—; and L, if present, is covalently bonded to Z through the functionalizable O or N atom.

In specific aspects, the disclosed subject matter relates to methods for inducing an analgesic drug effect. In other specific aspects, the disclosed subject matter relates to methods of treating and preventing pain.

In other aspects, disclosed are compounds comprising Formula I:

Z—Y (II)

or a pharmaceutically acceptable salt thereof, wherein: Z is an anesthetic having a functionalizable O or N atom; and Y is —C(O)—(CH₂)₁₋₆CO₂H, wherein Y is covalently bounded to Z through the functionalizable O or N atom.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIG. 1 depicts FITC, fluorescein-PEG₇₅₀, and glucose-UDP-fluorescein permeability across an in vitro blood-brain barrier model.

FIGS. 2A-2B depict in vivo peripheral nerve permeability. FIG. 2A shows representative fluorescent photomicrographs of sciatic nerves and surrounding epineurium 4 hours after perineural injection of FITC and glucose-UDP-fluorescein conjugate. FIG. 2B shows the relationship between normalized mean fluorescence intensity and normalized distance from the surface of the nerve in rats injected with FITC and glucose-UDP-fluorescein conjugate. n=3.

FIG. 3 depicts the synthesis of a galactose-capsaicin prodrug.

FIGS. 4A-4F depict sciatic nerve blockade with different dosages of free capsaicin and galactose-capsaicin prodrugs. FIG. 4A shows the effects of dose on frequency of successful nociceptive axon and motor axon blockade. FIG. 4B shows the effects of dose on duration of nociceptive axon blockade (i.e. thermal nociception blockade). FIGS. 4C-4F show the effects of dose on the frequency of side effects, including acute respiratory distress, seizure, contralateral nociceptive axon block, and irreversible nociceptive axon blockade. n=4. Data are mean±SD.

FIG. 5 shows co-administration of increasing dose of glucose with 2 mg prodrug results in a decrease in nociceptive axon block duration.

FIG. 6 depicts tissue reaction to capsaicin and prodrug 14 days after perineural injection. Representative photographs of the dissected injection site, followed by representative H&E-stained thick sections of muscles and adjacent loose connective tissue, and toluidine blue-stained semi-thin epon-embedded sciatic nerve sections are shown. N: epimysial connective tissue, M: muscle. Scale bar: 200 m.

FIG. 7 depicts the size of aggregates formed by GalA-CAP prodrug formulated with 2.5% (w/v) Tween 20 in the PBS buffer.

FIGS. 8A-8P depict an assessment of nociceptive axon blockade (FIGS. 8A-8H) and motor function impairment (FIGS. 8I-8P) in rats post-administration with 3 mg of GalA-CAP prodrug formulated with 2.5% (w/v) Tween 20 in 0.3 mL PBS buffer. Data in FIGS. 8A-8H are means±SD.

FIGS. 9A-9D depict a time course of the nociceptive axon blockade of rats injected with 2 mg capsaicin formulated with 2.5% (w/v) Tween 20 in 0.3 mL PBS buffer.

FIGS. 10A-10D depict a time course of the nociceptive axon blockade of rats injected with 3 mg capsaicin formulated with 4% (w/v) Tween 20 in 0.3 mL PBS buffer.

FIG. 11 depicts representative photos of injection sites. Red arrow denotes the sciatic nerve.

FIGS. 12A-12B depict representative histology of dissected sciatic nerves and surrounding muscle tissue. H&E-stained sections of muscle tissue (FIG. 12A) and toluidine blue-stained sections of sciatic nerves (FIG. 12B). Tissues harvested at 4- and 14-days post-injection were injected 2 mg capsaicin and GalA-CAP prodrug, whereas those harvested at 28 days post-injection were injected 3 mg capsaicin and GalA-CAP prodrug. Red arrow denotes perifascicular internalization of nucleus. M: Muscle; N: Nerve region; Inf: Inflammation. Scale bar: 200 m.

FIG. 13 depicts representative TEM images of dissected sciatic nerves and the diameter of axons. Red arrows denote unmyelinated fibers. The nerve tissues were harvested at 28 days post-injection, and the nerve from rats without injections were harvested at the same time. Scale bar: 2 m. n=200 fibers for diameter measurement. Data shown mean±SD. *P<0.05, ns-not significant, analysis of variance (ANOVA), n=4.

FIG. 14 depicts the distribution of C-fiber diameters. Red arrows denote that the rats had an irreversible nociceptive nerve blockade. n=200 fibers for each group. Data are means±SD.

FIGS. 15A-15F depict inhibition of the prodrug uptake by glucose and KL-11743. FIG. 15A shows representative time courses of thermal latency in rats co-injected with 2 mg GalA-CAP prodrug and different doses of glucose. FIG. 15B shows time courses of thermal latency of the ipsilateral side of rats co-injected with 2 mg GalA-CAP prodrug and different doses of KL-11743. FIG. 15C shows the average duration of nociceptive axon blockade in rats co-injected with 2 mg GalA-CAP prodrug or capsaicin with different doses of inhibitors. FIG. 15D shows structures of GalA-CAP prodrug and Gly-CAP prodrug. FIGS. 15E-15F show a comparison on frequency of successful axon blockade (FIG. 15E) and duration of nociceptive axon blockade (FIG. 15F) between 3 mg GalA-CAP prodrug and 3 mg Gly-CAP prodrug. Data shown are mean±SD. *P<0.05, **P<0.01, ***P<0.001, ****P<0.0001, ns: not significant, analysis of variance (ANOVA).

FIG. 16 depicts a time course of the nociceptive axon blockade of rats injected with 100 μM KL-11743 formulated with 2.5% (w/v) Tween 20 in 0.3 mL PBS buffer.

FIG. 17 depicts a time course of the nociceptive axon blockade of rats injected with 2 mg capsaicin with 100 μM KL-11743 formulated with 2.5% (w/v) Tween 20 in 0.3 mL PBS buffer.

FIG. 18 depicts hydrolysis kinetics of Gly-CAP prodrug in DI water at 37° C. determined by UPLC. The concentration of Gly-CAP prodrug is 1 mg/mL. The solution was stirred at 200 rpm and kept at 37° C.

FIG. 19 depicts a time course of the nerve blockade of rats injected with 3 mg Gly-CAP prodrug (formulated with 2.5% (w/v) Tween 20 in 0.3 mL PBS buffer) on the injected side (left) and contralateral side (right). Data are mean±SD.

FIGS. 20A-20D depict in vivo nerve penetration of FITC, Gal-FL, Glc-UDP-FL, and PEG750-FL. FIG. 20A shows representative confocal images of sciatic nerves and surrounding tissues 4 h after injecting FITC, Gal-FL, Gal-FL® 100 μM KL-11743, Glc-UDP-FL, Glc-UDP-FL@ 100 μM KL-11743, and PEG₇₅₀-FL. The image taken from higher laser intensity were inserted on the left top for groups of Gal-FL@ 100 μM KL-11743, Glc-UDP-FL@ 100 μM KL-11743, and PEG₇₅₀-FL, due to poor signals collected using the same camera settings. The contour of nerves was circled in red dash lines. Scale bar: 100 μm. FIG. 20B shows mean fluorescent intensity in epineurium and perineurium. FIG. 20C shows mean fluorescent intensity in nerves. FIG. 20D shows the relationship between mean fluorescent intensity and normalized distance from the epineurium/perineurium of the nerve. n=3, *P<0.05, **P<0.01, ***P<0.001, ****P<0.0001. Data are means±SD.

FIGS. 21A-21J depict enhanced efficacy and reduced side effects due to sustained release of capsaicin from the less active prodrug. FIG. 21A shows structures of GalA-CAP prodrug, Gly-CAP prodrug, and Gal-CAP prodrug. FIGS. 21B-21C show a comparison on frequency of successful axon blockade (FIG. 21B) and duration of nociceptive axon blockade (FIG. 21C) between 3 mg GalA-CAP prodrug, 3 mg Gly-CAP prodrug, 3 mg Gal-CAP prodrug, and 3 mg capsaicin. Data shown are mean±SD. *P<0.05, **P<0.01, ***P<0.001, ****P<0.0001, analysis of variance (ANOVA). FIGS. 21D-21J show molecular docking of capsaicin and prodrugs. Molecular docking of capsaicin and prodrugs. FIG. 21D shows published binding mode of capsaicin with TRPV1 (generated from PDB code 7LPE). The atoms of capsaicin are colored as follows: carbon, green; oxygen, red; nitrogen, blue. The cartoon shows the protein and key residues are colored as follows: carbon, grey; oxygen, red; nitrogen, blue. Yellow dotted lines indicate H-bonds, and the red dotted lines represent π-π stacking interactions. FIG. 21E, FIG. 21G, and FIG. 21I show proposed binding modes of GalA-CAP, Gly-CAP, and Gal-CAP prodrug with TRPV1. FIG. 21F, FIG. 21H, and FIG. 21J show superposed docking poses of capsaicin and GalA-CAP, Gly-CAP, or Gal-CAP prodrug. The atoms of prodrugs are colored as follows. GalA-CAP: carbon, cyan; oxygen, red; nitrogen, blue. Gly-CAP: carbon, purple; oxygen, red; nitrogen, blue. Gal-CAP: carbon, pink; oxygen, red; nitrogen, blue.

FIGS. 22A-22H depict a time course of the nerve blockade of rats injected with 3 mg Gal-CAP prodrug (formulated with 2.5% (w/v) Tween 20 in 0.3 mL PBS buffer) on the injected side and contralateral side. Data are mean±SD.

FIGS. 23A-23B depict Licking frequency (FIG. 23A) and licking duration (FIG. 23B) during the first 5 min after intraplantar application of 0.1% (w/v) capsaicin, 0.05% (w/v) capsaicin, 0.1% (w/v) GalA-CAP prodrug, 0.1% (w/v) Gal-CAP prodrug and 1×PBS buffer. *P<0.05, ***P<0.001 analysis of variance (ANOVA), n=4.

FIGS. 24A-24C depict sciatic nerve blockade with different dosages of free bupivacaine and glucuronic acid-linked bupivacaine (i.e., GluA-Bup). FIGS. 24A-24C show the effects of dose on frequency of successful nociceptive axon and motor axon blockade (FIG. 24A), duration of nociceptive axon blockade (i.e., thermal nociception blockade) (FIG. 24B), and duration of motor axon blockade (FIG. 24C). n=4. Data are mean±SD.

FIG. 25 depicts tissue reaction to 0.3 mL 17.3 mM of glucuronic acid-linked bupivacaine 4 days after perineural injection. Representative photographs of the dissected injection site, followed by representative H&E-stained thick sections of muscles and adjacent loose connective tissue. N: Nerve, M: muscle. Scale bar: 500 μm.

FIGS. 26A-26B depict in vivo peripheral nerve permeability. FIG. 26A shows representative fluorescent photomicrographs of sciatic nerves and surrounding epineurium 4 hours after perineural injection of FITC and 5 FAM. FIG. 26B shows the relationship between normalized mean fluorescence intensity and normalized distance from the surface of the nerve in rats injected with FITC and 5 FAM. n=3.

FIGS. 27A-27B depict sciatic nerve blockade with different dosages of free capsaicin and carboxylic acid derivative of capsaicin (i.e., capsaicin-COOH). FIGS. 27A-27B show the effects of dose on frequency of successful nociceptive axon and motor axon blockade (FIG. 27A) and duration of nociceptive axon blockade (i.e., thermal nociception blockade) (FIG. 27B). n=4. Data are mean±SD.

FIG. 28 depicts tissue reaction to 3.28 μmol of carboxylic acid derivative of capsaicin 14 days after perineural injection. Representative photographs of the dissected injection site, followed by representative H&E-stained thick sections of muscles and adjacent loose connective tissue, and toluidine blue-stained semi-thin epon-embedded sciatic nerve sections are shown. N: epimysial connective tissue, M: muscle. Scale bar: 200 μm.

### DETAILED DESCRIPTION

The materials, compounds, compositions, and methods described herein may be understood more readily by reference to the following detailed description of specific aspects of the disclosed subject matter, the Figures, and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### Definitions

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an inhibitor” includes mixtures of two or more such inhibitors, and the like.

“Optional” or “optionally” means that the subsequently described event or circumstance can or cannot occur, and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

By “reduce” or other forms of the word, such as “reducing” or “reduction,” is meant lowering of an event or characteristic (e.g., pain). It is understood that this is typically in relation to some standard or expected value, in other words it is relative, but that it is not always necessary for the standard or relative value to be referred to. For example, “reduces pain” means decreasing the amount of pain in a subject relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “treatment” refers to the medical management of a patient with the intent to cure, ameliorate, stabilize a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aldehyde” as used herein is represented by the formula C(O)H. Throughout this specification “C(O)” is a short hand notation for C═O.

The terms “amine” and “amino” are art-recognized and refer to both unsubstituted and substituted amines, e.g., a moiety that can be represented by the general formula:

—NR⁹R¹⁰ or N^{R9}R¹⁰R′¹⁰, wherein R⁹, R¹⁰, and R′¹⁰ each independently represent a hydrogen, an alkyl, an alkenyl, -(CH₂)m-R′⁸ or R⁹ and R¹⁰ taken together with the N atom to which they are attached complete a heterocycle having from 4 to 8 atoms in the ring structure; R′⁸ represents an aryl, a cycloalkyl, a cycloalkenyl, a heterocycle or a polycycle; and m is zero or an integer in the range of 1 to 8. In some embodiments, only one of R⁹ or R¹⁰ can be a carbonyl, e.g., R⁹, R¹⁰ and the nitrogen together do not form an imide. In some embodiments, the term “amine” does not encompass amides, e.g., wherein one of R⁹ and R¹⁰ represents a carbonyl. In some embodiments, R⁹ and R¹⁰ (and optionally R′¹⁰) each independently represent a hydrogen, an alkyl or cycloakly, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R⁹ and R¹⁰ is an alkyl group.

The term “amido” is art-recognized as an amino-substituted carbonyl and includes a moiety that can be represented by the general formula —CONR⁹R¹⁰ wherein R⁹ and R¹⁰ are as defined above.

The term “carboxylic acid” as used herein is represented by the formula —C(O)OH. A “carboxylate” as used herein is represented by the formula —C(O)O—.

The term “ester” as used herein is represented by the formula OC(O)A¹ or C(O)OA1, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A1 and A2 can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “hydroxyl” as used herein is represented by the formula —OH.

The term “nitro” as used herein is represented by the formula —NO₂.

The term “cyano” as used herein is represented by the formula —CN

The term “azido” as used herein is represented by the formula —N₃.

The term “sulfonyl” is used herein to refer to the sulfo-oxo group represented by the formula —S(O)₂A¹, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH₂.

The term “thiol” as used herein is represented by the formula —SH.

It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

The term “anesthetic” refers to a compound capable of altering perception of pain in a subject or patient. Anesthetics can include, but are not limited to: Acetaminophen; Alfentanil Hydrochloride; Aminobenzoate Potassium; Aminobenzoate Sodium; Anidoxime; Anileridine; Anileridine Hydrochloride; Anilopam Hydrochloride; Anirolac; Antipyrine; Aspirin; Benoxaprofen; Benzydamine Hydrochloride; Bicifadine Hydrochloride; Brifentanil Hydrochloride; Bromadoline Maleate; Bromfenac Sodium; Buprenorphine Hydrochloride; Butacetin; Butixirate; Butorphanol; Butorphanol Tartrate; Carbamazepine; Carbaspirin Calcium; Carbiphene Hydrochloride; Carfentanil Citrate; Ciprefadol Succinate; Ciramadol; Ciramadol Hydrochloride; Clonixeril; Clonixin; Codeine; Codeine Phosphate; Codeine Sulfate; Conorphone Hydrochloride; Cyclazocine; Dexoxadrol Hydrochloride; Dexpemedolac; Dezocine; Diflunisal; Dihydrocodeine Bitartrate; Dimefadane; Dipyrone; Doxpicomine Hydrochloride; Drinidene; Enadoline Hydrochloride; Epirizole; Ergotamine Tartrate; Ethoxazene Hydrochloride; Etofenamate; Eugenol; Fenoprofen; Fenoprofen Calcium; Fentanyl Citrate; Floctafenine; Flufenisal; Flunixin; Flunixin Meglumine; Flupirtine Maleate; Fluproquazone; Fluradoline Hydrochloride; Flurbiprofen; Hydromorphone Hydrochloride; Ibufenac; Indoprofen; Ketazocine; Ketorfanol; Ketorolac Tromethamine; Letimide Hydrochloride; Levomethadyl Acetate; Levomethadyl Acetate Hydrochloride; Levonantradol Hydrochloride; Levorphanol Tartrate; Lofemizole Hydrochloride; Lofentanil Oxalate; Lorcinadol; Lomoxicam; Magnesium Salicylate; Mefenamic Acid; Menabitan Hydrochloride; Meperidine Hydrochloride; Meptazinol Hydrochloride; Methadone Hydrochloride; Methadyl Acetate; Methopholine; Methotrimeprazine; Metkephamid Acetate; Mimbane Hydrochloride; Mirfentanil Hydrochloride; Molinazone; Morphine Sulfate; Moxazocine; Nabitan Hydrochloride; Nalbuphine Hydrochloride; Nalmexone Hydrochloride; Namoxyrate; Nantradol Hydrochloride; Naproxen; Naproxen Sodium; Naproxol; Nefopam Hydrochloride; Nexeridine Hydrochloride; Noracymethadol Hydrochloride; Ocfentanil Hydrochloride; Octazamide; Olvanil; Oxetorone Fumarate; Oxycodone; Oxycodone Hydrochloride; Oxycodone Terephthalate; Oxymorphone Hydrochloride; Pemedolac; Pentamorphone; Pentazocine; Pentazocine Hydrochloride; Pentazocine Lactate; Phenazopyridine Hydrochloride; Phenyramidol Hydrochloride; Picenadol Hydrochloride; Pinadoline; Pirfenidone; Piroxicam Olamine; Pravadoline Maleate; Prodilidine Hydrochloride; Profadol Hydrochloride; Propirarn Fumarate; Propoxyphene Hydrochloride; Propoxyphene Napsylate; Proxazole; Proxazole Citrate; Proxorphan Tartrate; Pyrroliphene Hydrochloride; Remifentanil Hydrochloride; Salcolex; Salethamide Maleate; Salicylamide; Salicylate Meglumine; Salsalate; Sodium Salicylate; Spiradoline Mesylate; Sufentanil; Sufentanil Citrate; Talmetacin; Talniflumate; Talosalate; Tazadolene Succinate; Tebufelone; Tetrydamine; Tifurac Sodium; Tilidine Hydrochloride; Tiopinac; Tonazocine Mesylate; Tramadol Hydrochloride; Trefentanil Hydrochloride; Trolamine; Veradoline Hydrochloride; Verilopam Hydrochloride; Volazocine; Xorphanol Mesylate; Xylazine Hydrochloride; Zenazocine Mesylate; Zomepirac Sodium; Zucapsaicin.

A “pharmaceutically acceptable” component is one that is suitable for use with humans and/or animals without undue adverse side effects (such as toxicity, irritation, and allergic response) commensurate with a reasonable benefit/risk ratio.

“Pharmaceutically acceptable salt” refers to a salt that is pharmaceutically acceptable and has the desired pharmacological properties. Such salts include those that may be formed where acidic protons present in the compounds are capable of reacting with inorganic or organic bases. Suitable inorganic salts include those formed with the alkali metals, e.g., sodium, potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). When two acidic groups are present, a pharmaceutically acceptable salt may be a mono-acid-mono-salt or a di-salt; similarly, where there are more than two acidic groups present, some or all of such groups can be converted into salts.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to pain, an effective amount comprises an amount sufficient to reduce inflammation, reduce discomfort, and/or otherwise provide pain relief to a patient. In some embodiments, an effective amount is an amount sufficient to prevent or delay occurrence and/or recurrence. An effective amount can be administered in one or more doses.

Effective amounts of a compound or composition described herein for treating a mammalian subject can include about 0.1 to about 1000 mg/Kg of body weight of the subject/day, such as from about 1 to about 100 mg/Kg/day, especially from about 10 to about 100 mg/Kg/day. The doses can be acute or chronic. A broad range of disclosed composition dosages are believed to be both safe and effective.

Reference will now be made in detail to specific aspects of the disclosed materials, compounds, compositions, articles, and methods, examples of which are illustrated in the accompanying Examples and Figures.

### Compounds

In one aspect, provided is a compound of Formula I:

Z-L-X (I)

or pharmaceutically acceptable salt thereof, wherein: Z is an anesthetic having a functionalizable o or N atom; L is an optional divalent linker; X is a monosaccharide, a disaccharide, or H; L is covalently bonded to X by —O—; and L is covalently bonded to Z through the functionalizable 0 or N atom.

In other aspects, disclosed are compounds comprising Formula I:

Z-Y (II)

or a pharmaceutically acceptable salt thereof, wherein: Z is an anesthetic having a functionalizable o or N atom; and Y is —C(O)—(CH₂)₁₋₆CO₂H, wherein Y is covalently bounded to Z through the functionalizable O or N atom.

GLUT1 facilitates the transport of various sugars with different binding affinities, including glucose, galactose, mannose, glucosamine, and dehydroascorbic acid (DHA) [19-21]. Among these, DHA exhibits the highest affinity (Km: 1.1 mmol/L), followed by glucosamine (Km: 2.5 mmol/L), glucose (Km: 5 mmol/L), mannose (Km: 5 mmol/L), and galactose (Km: 15 mmol/L) (Km (mmol/L) is an indicator of transporter protein affinity for sugar molecules; a low Km value suggests a high affinity). In some aspects, sugar-capsaicin prodrugs can be synthesized using any of the described sugars or other suitable sugars.

In some aspects, X is a monosaccharide. In some aspects, X is a disaccharide. In some aspects, X is hydrogen. It is considered that, when X is hydrogen, L must be present.

In some aspects, X is selected from the group consisting of glucose, galactose, mannose, glucosamine, dehydroascorbic acid, glucuronic acid, N-acetyl-glycosamine, L-iduronic acid, N-sulfo-D-glucosamine, glyceraldehyde, xylose, ribose, sucrose, lactose, trehalose, maltose, cellobiose, and raffinose. In some aspects, X is glucose. In some aspects, X is galactose. In some aspects, X is mannose. In some aspects, X is glucosamine. In some aspects, X Is dehydroascorbic acid. In some aspects, X is N-acetyl-glycosamine. In some aspects, X is L-iduronic acid. In some aspects, X is N-sulfo-D-glucosamine. In some aspects, X is glyceraldehyde. In some aspects, X is xylose. In some aspects, X is ribose. In some aspects, X is sucrose. In some aspects, X is lactose. In some aspects, X is trehalose. In some aspects, X is maltose. In some aspects, X is cellobiose. In some aspects, X is raffinose.

The linker moiety (L) plays a role in determining prodrug properties, such as stability, pharmacokinetics, bioavailability to target sites, organ distribution, and ultimately affects therapeutic efficacy and potential side effects [28]. Chronic pain is often associated with persistent inflammation, creating a pathological microenvironment determined by pro-inflammatory mediators (e.g., oxidative stress, acidic pH, and overexpressed enzymes) [29, 30]. To optimize therapeutic outcomes and minimize adverse effects, sugar-capsaicin prodrugs can be synthesized with different linkers, and the effects of linkers on nerve signaling blockade and associated side effects can be evaluated. Different linkers exhibit different responses to the pathological environments of inflamed tissues [31]. Specifically, carbonate, ester, and amide linkers are enzymatically degradable, while ester and ketal linkers are acid-labile. The thioketal linker is sensitive to reactive oxygen species (ROS) [32].

In some aspects, L is a hydrolysable divalent linker. In some aspects, L is a linker selected from the group consisting of —SO₂, —SO₂R′; SO₂R′R″, —SO₂NR′R″; —SO₂NR′R″C(═O); —NR′SO₂R″; —R′SO₂NR′R′; —C(═O); —C(═O)R′; —OC(═O)R′; —OC(═O)R″″C(═O)O—; —C(═O)R′C(═O)—; —C(═O)NR′R″; —NR′C(═O)R″; —NR′C(═O)R″″C(═O); —OR′; —NR′R″; -SR′; —N₃—C(═O)OR′; —O(CR′R″)ᵣC(═O)R′; —O(CR′R″)ᵣNR″C(═O)R′; —O(CR′R″)ᵣNR″SO₂R′; —OC(═O)NR′R″; —NR′C(═O)OR″; and substituted or unsubstituted C₁-C₆ aliphatic alkyl; wherein R′, R″, and R′″ are individually selected from hydrogen; substituted or unsubstituted alkyl; substituted or unsubstituted alkenyl; substituted or unsubstituted ether; substituted or unsubstituted cycloalkyl; substituted or unsubstituted heterocyclyl; substituted or unsubstituted cycloalkenyl, substituted or unsubstituted aryl, substituted or unsubstituted heteroaryl, substituted or unsubstituted arylalkyl, substituted or unsubstituted heteroalkyl, substituted or unsubstituted alkylheteroaryl, or substituted or unsubstituted amine; R″″ is selected from substituted or unsubstituted alkyl; substituted or unsubstituted alkenyl; substituted or unsubstituted ether; substituted or unsubstituted cycloalkyl; substituted or unsubstituted heterocyclyl; substituted or unsubstituted cycloalkenyl, substituted or unsubstituted aryl, substituted or unsubstituted heteroaryl, substituted or unsubstituted arylalkyl, substituted or unsubstituted heteroalkyl, substituted or unsubstituted alkylheteroaryl, or substituted or unsubstituted amine; and r is an integer from 1 to 6.

In some aspects, L is —O—C(═O)—. In some aspects, L is —OC(═O)R″″C(═O)O—. In some aspects, L is —SO₂. In some aspects, L is —SO₂R′. In some aspects, L is SO₂R′R″. In some aspects, L is —SO₂NR′R″. In some aspects, L is —SO₂NR′R″C(═O). In some aspects, L is —NR′SO₂R″. In some aspects, L is —R′SO₂NR′R′″. In some aspects, L is —C(═O). In some aspects, L is —C(═O)R′. In some aspects, L is —OC(═O)R′. In some aspects, L is —C(═O)NR′R″. In some aspects, L is —NR′C(═O)R″. In some aspects, L is —NR′C(═O)R″″C(═O). In some aspects, L is —OR′. In some aspects, L is —NR′R″. In some aspects, L is —SR′. In some aspects, L is —N₃—C(═O)OR′. In some aspects, L is —O(CR′R″)ᵣC(═O)R′. In some aspects, L is —O(CR′R″)ᵣNR″C(═O)R′. In some aspects, L is —O(CR′R″)ᵣNR″SO₂R′. In some aspects, L is —OC(═O)NR′R″. In some aspects, L is —NR′C(═O)OR″. In some aspects, L is substituted or unsubstituted C₁-C₆ aliphatic alkyl. In some aspects, L is succinic acid. In some aspects, L is —C(═O)R′C(═O)—, and R′ is C₁₋₄ alkyl.

In some aspects, R′ is hydrogen. In some aspects, R′ is substituted or unsubstituted alkyl. In some aspects, R′ is substituted or unsubstituted alkenyl. In some aspects, R′ is substituted or unsubstituted ether. In some aspects, R′ is substituted or unsubstituted cycloalkyl. In some aspects, R′ is substituted or unsubstituted heterocyclyl. In some aspects, R′ is substituted or unsubstituted cycloalkenyl. In some aspects, R′ is substituted or unsubstituted aryl. In some aspects, R′ is substituted or unsubstituted heteroaryl. In some aspects, R′ is substituted or unsubstituted arylalkyl. In some aspects, R′ is substituted or unsubstituted heteroalkyl. In some aspects, R′ is substituted or unsubstituted alkylheteroaryl. In some aspects, R′ is substituted or unsubstituted amine.

In some aspects, R″ is hydrogen. In some aspects, R″ is substituted or unsubstituted alkyl. In some aspects, R″ is substituted or unsubstituted alkenyl. In some aspects, R″ is substituted or unsubstituted ether. In some aspects, R″ is substituted or unsubstituted cycloalkyl. In some aspects, R″ is substituted or unsubstituted heterocyclyl. In some aspects, R″ is substituted or unsubstituted cycloalkenyl. In some aspects, R″ is substituted or unsubstituted aryl. In some aspects, R″ is substituted or unsubstituted heteroaryl. In some aspects, R″ is substituted or unsubstituted arylalkyl. In some aspects, R″ is substituted or unsubstituted heteroalkyl. In some aspects, R″ is substituted or unsubstituted alkylheteroaryl. In some aspects, R″ is substituted or unsubstituted amine.

In some aspects, R′″ is hydrogen. In some aspects, R′″ is substituted or unsubstituted alkyl. In some aspects, R′″ is substituted or unsubstituted alkenyl. In some aspects, R′ is substituted or unsubstituted ether. In some aspects, R′ is substituted or unsubstituted cycloalkyl. In some aspects, R′ is substituted or unsubstituted heterocyclyl. In some aspects, R′ is substituted or unsubstituted cycloalkenyl. In some aspects, R′ is substituted or unsubstituted aryl. In some aspects, R′″ is substituted or unsubstituted heteroaryl. In some aspects, R′″ is substituted or unsubstituted arylalkyl. In some aspects, R′″ is substituted or unsubstituted heteroalkyl. In some aspects, R′ is substituted or unsubstituted alkylheteroaryl. In some aspects, R′ is substituted or unsubstituted amine.

In some aspects, R″″ is substituted or unsubstituted alkyl. In some aspects, R″″ is substituted or unsubstituted alkenyl. In some aspects, R″″ is substituted or unsubstituted ether. In some aspects, R″″ is substituted or unsubstituted cycloalkyl. In some aspects, R″″ is substituted or unsubstituted heterocyclyl. In some aspects, R″″ is substituted or unsubstituted cycloalkenyl. In some aspects, R″″ is substituted or unsubstituted aryl. In some aspects, R″″ is substituted or unsubstituted heteroaryl. In some aspects, R″″ is substituted or unsubstituted arylalkyl. In some aspects, R″″ is substituted or unsubstituted heteroalkyl. In some aspects, R″″ is substituted or unsubstituted alkylheteroaryl. In some aspects, R″″ is substituted or unsubstituted amine.

In some aspects, r is 1. In some aspects, r is 2. In some aspects, r is 3. In some aspects, r is 4. In some aspects, r is 5. In some aspects, r is 6.

In some aspects, Y is —C(O)—(CH₂)_{I}CO₂H. In some aspects, Y is —C(O)—(CH₂)₂CO₂H. In some aspects, Y is —C(O)—(CH₂)₃CO₂H. In some aspects, Y is —C(O)—(CH₂)₄CO₂H. In some aspects, Y is —C(O)—(CH₂)₅CO₂H. In some aspects, Y is —C(O)—(CH₂)₆CO₂H. It is considered that Y can be linear or branched.

In some aspects, Z is selected from the group consisting of capsaicin, bupivacaine, tetracaine, lidocaine, benzocaine, procaine, prilocaine, cinchocaine, ropivacaine, tetrodotoxin, saxitoxin, resiniferatoxin, botulinum toxin, a TRPV1 activator (e.g., gingerol, shogaol, zingerone, eugenol, N-arachidonoyl-dopamine, anandamide) and analogs thereof. In some aspects, Z is capsaicin. In some aspects, Z is bupivacaine. In some aspects, Z is tetracaine. In some aspects, Z is lidocaine. In some aspects, Z is benzocaine. In some aspects, Z is procaine. In some aspects, Z is prilocaine. In some aspects, Z is cinchocaine. In some aspects, Z is ropivacaine. In some aspects, Z is tetrodotoxin. In some aspects, Z is saxitoxin. In some aspects, Z is resiniferatoxin. In some aspects, Z is botulinum toxin. In some aspects, Z is a TRPV1 activator. In some such aspects, Z is gingerol. In some such aspects, Z is shogaol. In some such aspects, Z is zingerone. In some such aspects, Z is eugenol. In some such aspects, Z is N-arachidonoyl-dopamine. In some such aspects, Z is anandamide.

In some aspects, Z has the formula:

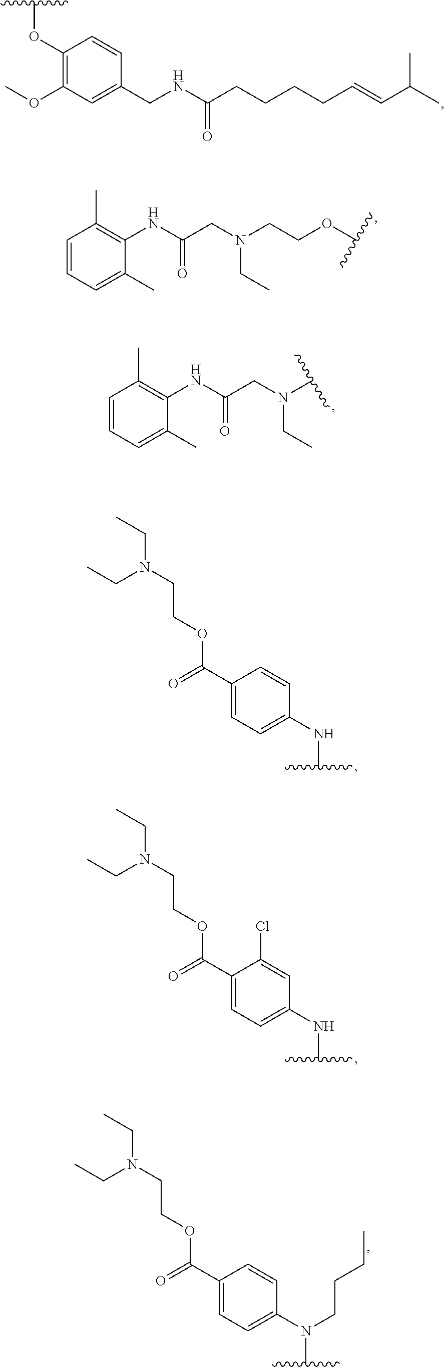

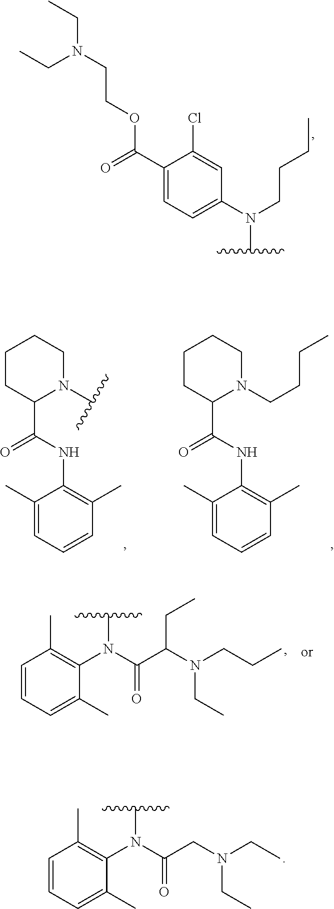 [SMILES: *N(CCCC)c1ccc(C(=O)OCCN(CC)CC)c(Cl)c1 ; *N1CCCCC1C(=O)Nc1c(C)cccc1C ; CCCCN1CCCCC1C(=O)Nc1c(C)cccc1C ; *N(C(=O)C(CC)N(CC)CCC)c1c(C)cccc1C ; *N(C(=O)CN(CC)CC)c1c(C)cccc1C]

In some aspects, the compound has the formula:

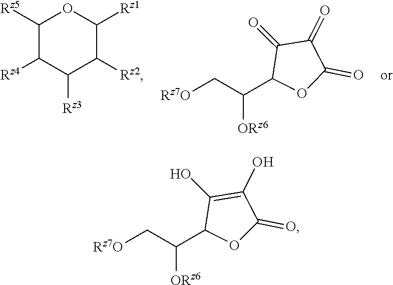

wherein: R^{z1} is OH, OC₁₋₃alkyl, NH-L-Z, or O-L-Z; R^{z}z is H, OH, OR^{d}, OC₁₋₃alkyl, NHC(═O)CH₃, NHC(═O)CH₂-L-Z, NH-L-Z, O-L-Z, or -L-Z; R^{z3} is H, OH, OR^{d}, OC₁₋₃alkyl, NHC(═O)CH₃, NHC(═O)CH₂-L-Z, NH-L-Z, O-L-Z, or -L-Z; R^{z4} is H, OH, OR^{d}, OC₁₋₃alkyl, NHC(═O)CH₃, NHC(═O)CH₂-L-Z, NH-L-Z, O-L-Z, or -L-Z; R^{z}s is H, CH₃, CH₂OR^{d}, CH₂OH, CH₂OC₁₋₃alkyl, COOH, or -L-Z; R^{z6} is H or -L-Z; R^{z7} is H or -L-Z; R^{d} has the formula:

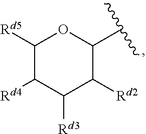

R^{d2} is H, OH, OC₁₋₃alkyl, NHC(═O)CH₃, NHC(═O)CH₂-L-Z, NH-L-Z, O-L-Z, or -L-Z; R^{d3} is H, OH, OC₁₋₃alkyl, NHC(═O)CH₃, NHC(═O)CH₂-L-Z, NH-L-Z, O-L-Z, or -L-Z; R^{d4} is H, OH, OC₁₋₃ alkyl, NHC(═O)CH₃, NHC(═O)CH₂-L-Z, NH-L-Z, O-L-Z, or -L-Z; R^{d5} is H, CH₃, CH₂OH, CH₂OC₁₋₃alkyl, COOH, or -L-Z; with the proviso that only one -L-Z group is present.

In some aspects, the compound has the formula:

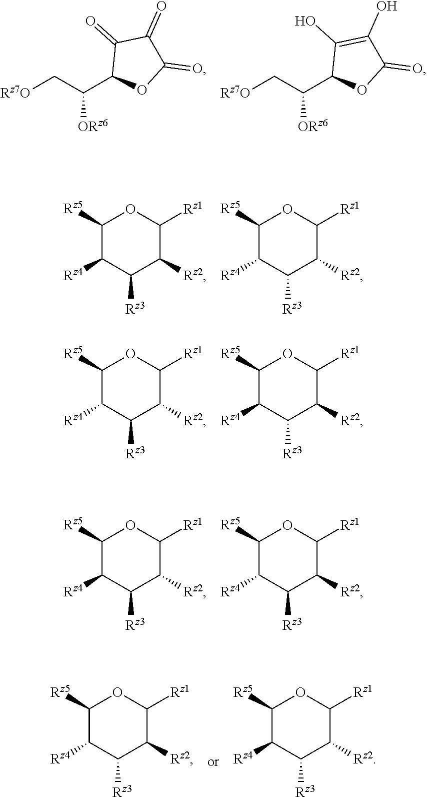

In some aspects, R^{d} has the formula:

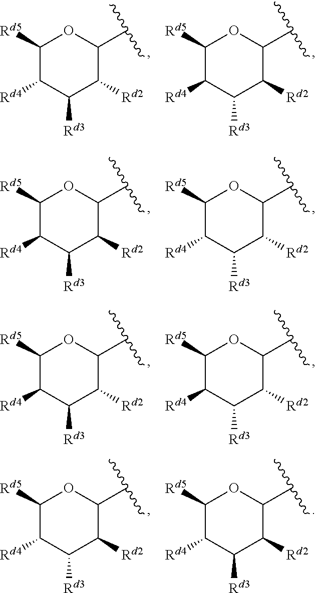

In some aspects, R^{Z1} is OH; R^{Z2} is OH; R^{Z3} is OH; R^{Z} is OH; and R^{Z5} is CH₂O-L-Z.

In some aspects, R^{Z1} is OH; R^{Z2} is OH; R^{Z3} is OH; R¹ is OH; and R^{Z5} is -L-Z.

In some aspects, R^{Z1} is OH; R^{Z2} is OH; R^{Z3} is OH; R¹ is OH; R^{Z5} is -L-Z.

In some aspects, R^{Z7} is OH; and R^{Z6} is -L-Z.

In some aspects, R^{Z1} is OH; R^{Z2} is NH-L-Z; R^{Z3} is OH; R^{Z4} is OH; and R^{Z5} is CH₂OH.

In some aspects, R^{Z1} is OH; R^{Z2} is OH; R^{Z3} is OH; R^{Z4} is NH-L-Z; and R^{Z5} is CH₂OH.

In some aspects, -L-X is —C(═O)[CH₂]ₙCOOH or —C(═O)[CH₂]ₙCONH₂, wherein n is 1-6, preferably 2-4, more preferably 2-3.

In some aspects, R^{d2} is OH; R^{d3} is OH; R^{d4} is OH; and R^{d5} is CH₂OH.

In some aspects, the compound is selected from:

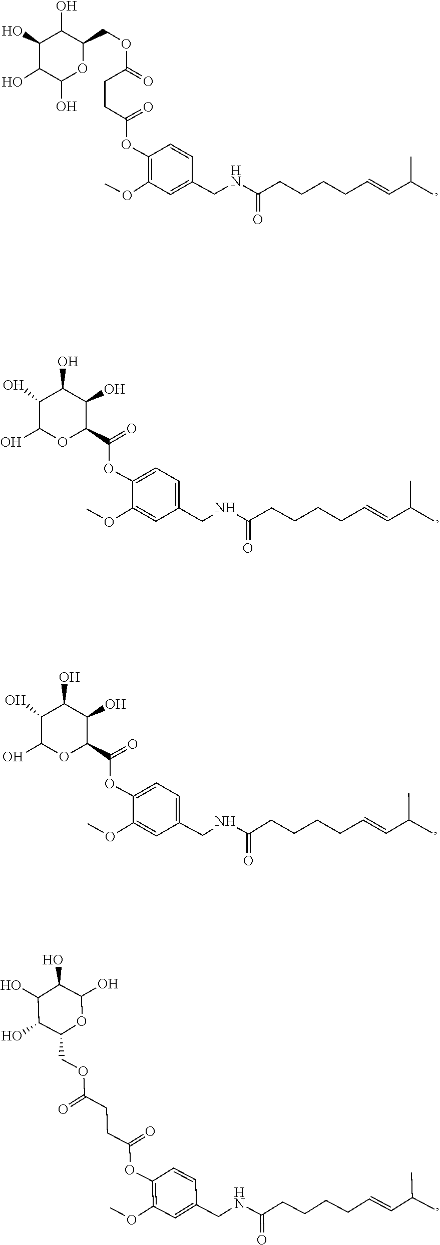 [SMILES: COc1cc(CNC(=O)CCCC/C=C/C(C)C)ccc1OC(=O)CCC(=O)OC[C@H]1OC(O)C(O)[C@@H](O)C1O ; COc1cc(CNC(=O)CCCC/C=C/C(C)C)ccc1OC(=O)[C@H]1OC(O)[C@H](O)[C@@H](O)[C@H]1O ; COc1cc(CNC(=O)CCCC/C=C/C(C)C)ccc1OC(=O)[C@H]1OC(O)[C@H](O)[C@@H](O)[C@H]1O ; COc1cc(CNC(=O)CCCC/C=C/C(C)C)ccc1OC(=O)CCC(=O)OC[C@H]1OC(O)[C@H](O)C(O)[C@H]1O]

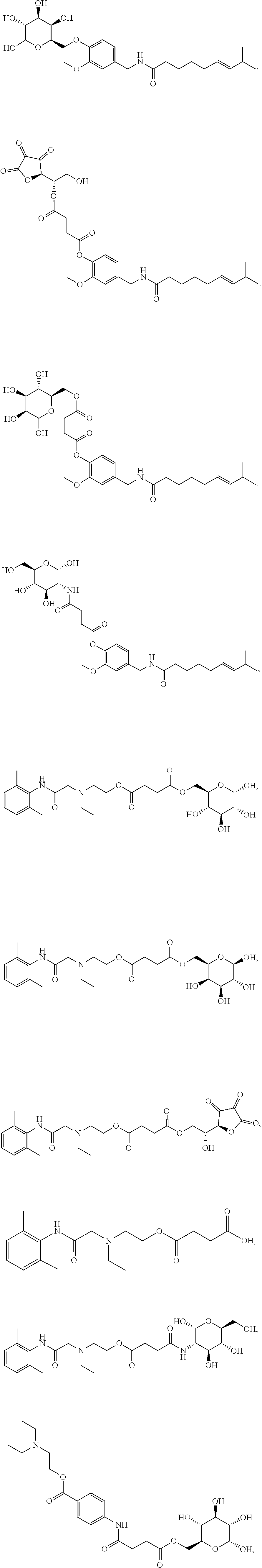

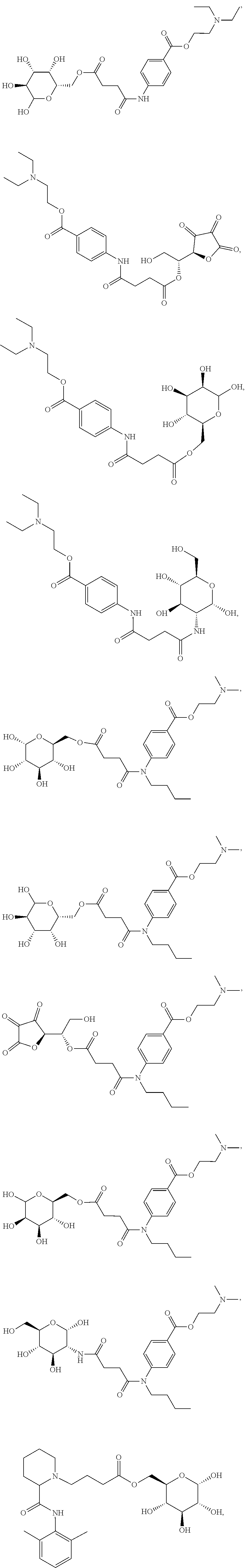

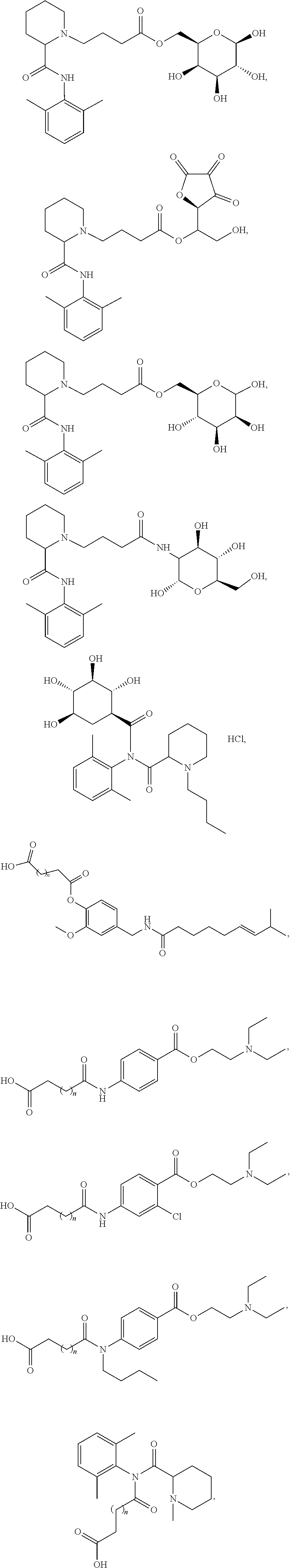

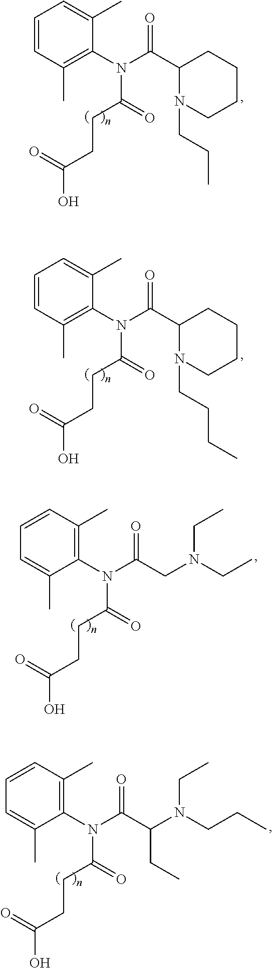

or pharmaceutically acceptable salts thereof.

In some aspects, the compound is

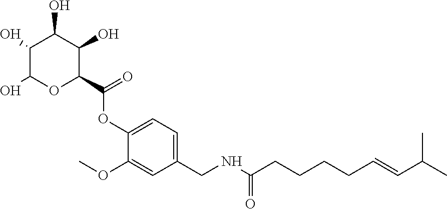 [SMILES: COc1cc(CNC(=O)CCCC/C=C/C(C)C)ccc1OC(=O)[C@H]1OC(O)[C@H](O)[C@@H](O)[C@H]1O]

or a pharmaceutically acceptable salt thereof.

In some aspects, the compound is

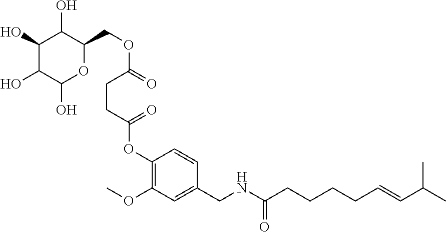 [SMILES: COc1cc(CNC(=O)CCCC/C=C/C(C)C)ccc1OC(=O)CCC(=O)OC[C@H]1OC(O)C(O)[C@@H](O)C1O]

or pharmaceutically acceptable salt thereof.

In some aspects, the compound has a high binding affinity to the GLUT1 transporter.

### Methods

In one aspect, provided is a method of inducing an analgesic drug effect in a subject in need thereof by administering any of the compounds disclosed herein. In some aspects, the compound induces a nociceptive-selective nerve blockade. In some aspects, the compound does not induce a motor nerve blockade.

In another aspect, provided is a method of treating and preventing pain in a subject in need thereof by using any of the disclosed compounds. In some aspects, the compound induces a nociceptive-selective nerve blockade. In some aspects, the compound does not induce a motor nerve blockade.

In yet another aspect, provided is a method for treating pain in a subject in need thereof comprising administering a composition comprising at least 1 wt. %, based upon the total weight of the composition, of any of the disclosed compounds, or a pharmaceutically acceptable salt or derivative thereof.

In yet still another aspect, provided is a method for preventing pain in a subject in need thereof comprising administering a composition comprising at least 1 wt. %, based upon the total weight of the composition, of any of the disclosed compounds, or a pharmaceutically acceptable salt thereof.

In some aspects, the methods described herein can be used to treat pain caused by: a physical cause such as burns, frostbite, physical injury (either blunt or penetrating), foreign bodies (including splinters, dirt, or debris), trauma, or ionizing radiation; a biological cause such as infection by a pathogen, an immune reaction due to hypersensitivity, or stress; or a chemical cause such as a chemical irritant, a toxin, surgical procedure.

In some aspects, the composition may comprise the compound of Formula I in an amount of at least 1 wt. %, at least 5 wt. %, at least 10 wt. %, at least 20 wt. %, at least 30 wt. %, at least 40 wt. %, at least 50 wt. %, at least 60 wt. %, at least 70 wt. %, or more based upon the total weight of the composition. In some embodiments, the composition may comprise the compound of Formula (I) in an amount from 1 to 70 wt. %, from 5 to 70 wt. %, from 10 to 70 wt. %, from 20 to 70 wt. %, from 30 to 70 wt. %, from 40 to 70 wt. %, from 50 to 70 wt. %, from 60 to 70 wt. %, from 1 to 60 wt. %, from 5 to 60 wt. %, from 10 to 60 wt. %, from 20 to 60 wt. %, from 30 to 60 wt. %, from 40 to 60 wt. %, from 50 to 60 wt. %, from 1 to 50 wt. %, from 5 to 50 wt. %, from 10 to 50 wt. %, from 20 to 50 wt. %, from 30 to 50 wt. %, from 40 to 50 wt. %, from 1 to 40 wt. %, from 5 to 40 wt. %, from 10 to 40 wt. %, from 20 to 40 wt. %, from 30 to 40 wt. %, from 1 to 30 wt. %, from 5 to 30 wt. %, from 10 to 30 wt. %, from 20 to 30 wt. %, from 1 to 20 wt. %, from 5 to 20 wt. %, from 10 to 20 wt. %, from 1 to 10 wt. %, from 5 to 10 wt. %, or 1 to 5 wt. %, based upon the total weight of the composition.

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and an excipient of some sort may be useful in a variety of medical and non-medical applications. For example, pharmaceutical compositions comprising an active compound and an excipient may be useful for the treatment or prevention of inflammation in a subject in need thereof.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, varoius gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar- agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard- filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

In some aspects, the disclosed methods comprise administering to a subject in need a compound of Formula I:

Z-L-X (I)

or pharmaceutically acceptable salt thereof, wherein: Z is an anesthetic having a functionalizable O or N atom; L is an optional divalent linker; X is a monosaccharide, a disaccharide, or H; L is covalently bonded to X by —O—; and L is covalently bonded to Z through the functionalizable O or N atom.

In some aspects, X is a monosaccharide. In some aspects, X is a disaccharide. In some aspects, X is hydrogen. It is considered that, when X is hydrogen, L must be present.

In some aspects, X is selected from the group consisting of glucose, galactose, mannose, glucosamine, dehydroascorbic acid glucuronic acid, N-acetyl-glycosamine, L-iduronic acid, N-sulfo-D-glucosamine, glyceraldehyde, xylose, ribose, sucrose, lactose, trehalose, maltose, cellobiose, and raffinose. In some aspects, X is glucose. In some aspects, X is galactose. In some aspects, X is mannose. In some aspects, X is glucosamine. In some aspects, X Is dehydroascorbic acid. In some aspects, X is N-acetyl-glycosamine. In some aspects, X is L-iduronic acid. In some aspects, X is N-sulfo-D-glucosamine. In some aspects, X is glyceraldehyde. In some aspects, X is xylose. In some aspects, X is ribose. In some aspects, X is sucrose. In some aspects, X is lactose. In some aspects, X is trehalose. In some aspects, X is maltose. In some aspects, X is cellobiose. In some aspects, X is raffinose

The linker moiety (L) plays a role in determining prodrug properties, such as stability, pharmacokinetics, bioavailability to target sites, organ distribution, and ultimately affects therapeutic efficacy and potential side effects [28]. Chronic pain is often associated with persistent inflammation, creating a pathological microenvironment determined by pro-inflammatory mediators (e.g., oxidative stress, acidic pH, and overexpressed enzymes) [29, 30]. To optimize therapeutic outcomes and minimize adverse effects, sugar-capsaicin prodrugs can be synthesized with different linkers, and the effects of linkers on nerve signaling blockade and associated side effects can be evaluated. Different linkers exhibit different responses to the pathological environments of inflamed tissues [31]. Specifically, carbonate, ester, and amide linkers are enzymatically degradable, while ester and ketal linkers are acid-labile. The thioketal linker is sensitive to reactive oxygen species (ROS) [32].

In some aspects, L is a hydrolysable divalent linker. In some aspects, L is a linker selected from the group consisting of —SO₂, —SO₂R′; SO₂R′R″, —SO₂NR′R″; —SO₂NR′R″C(═O); —NR′SO₂R″; —R′SO₂NR′R′; —C(═O); —C(═O)R′; —OC(═O)R′; —OC(═O)R″″C(═O)O—; —C(═O)R′C(═O)—; —C(═O)NR′R″; —NR′C(═O)R″; —NR′C(═O)R″″C(═O); —OR′; —NR′R″; -SR′; —N₃—C(═O)OR′; —O(CR′R″)ᵣC(═O)R′; —O(CR′R″)ᵣNR″C(═O)R′; —O(CR′R″)ᵣNR″SO₂R′; —OC(═O)NR′R″; —NR′C(═O)OR″; and substituted or unsubstituted C₁-C₆ aliphatic alkyl; wherein R′, R″, and R′″ are individually selected from hydrogen; substituted or unsubstituted alkyl; substituted or unsubstituted alkenyl; substituted or unsubstituted ether; substituted or unsubstituted cycloalkyl; substituted or unsubstituted heterocyclyl; substituted or unsubstituted cycloalkenyl, substituted or unsubstituted aryl, substituted or unsubstituted heteroaryl, substituted or unsubstituted arylalkyl, substituted or unsubstituted heteroalkyl, substituted or unsubstituted alkylheteroaryl, or substituted or unsubstituted amine; R″″ is selected from substituted or unsubstituted alkyl; substituted or unsubstituted alkenyl; substituted or unsubstituted ether; substituted or unsubstituted cycloalkyl; substituted or unsubstituted heterocyclyl; substituted or unsubstituted cycloalkenyl, substituted or unsubstituted aryl, substituted or unsubstituted heteroaryl, substituted or unsubstituted arylalkyl, substituted or unsubstituted heteroalkyl, substituted or unsubstituted alkylheteroaryl, or substituted or unsubstituted amine; and r is an integer from 1 to 6.

In some aspects, L is —O—C(═O)—. In some aspects, L is —OC(═O)R″″C(═O)O—. In some aspects, L is —SO₂. In some aspects, L is —SO₂R′. In some aspects, L is SO₂R′R″. In some aspects, L is —SO₂NR′R″. In some aspects, L is —SO₂NR′R″C(═O). In some aspects, L is —NR′SO₂R″. In some aspects, L is —R′SO₂NR′R′″. In some aspects, L is —C(═O). In some aspects, L is —C(═O)R′. In some aspects, L is —OC(═O)R′. In some aspects, L is —C(═O)NR′R″. In some aspects, L is —NR′C(═O)R″. In some aspects, L is —NR′C(═O)R″″C(═O). In some aspects, L is —OR′. In some aspects, L is —NR′R″. In some aspects, L is —SR′. In some aspects, L is —N₃—C(═O)OR′. In some aspects, L is —O(CR′R″)ᵣC(═O)R′. In some aspects, L is —O(CR′R″)ᵣNR″C(═O)R′. In some aspects, L is —O(CR′R″)ᵣNR″SO₂R′. In some aspects, L is —OC(═O)NR′R″. In some aspects, L is —NR′C(═O)OR″. In some aspects, L is substituted or unsubstituted C₁-C₆ aliphatic alkyl. In some aspects, L is succinic acid. In some aspects, L is —C(═O)R′C(═O)—, and R′ is C₁₋₄ alkyl.

In some aspects, R′ is hydrogen. In some aspects, R′ is substituted or unsubstituted alkyl. In some aspects, R′ is substituted or unsubstituted alkenyl. In some aspects, R′ is substituted or unsubstituted ether. In some aspects, R′ is substituted or unsubstituted cycloalkyl. In some aspects, R′ is substituted or unsubstituted heterocyclyl. In some aspects, R′ is substituted or unsubstituted cycloalkenyl. In some aspects, R′ is substituted or unsubstituted aryl. In some aspects, R′ is substituted or unsubstituted heteroaryl. In some aspects, R′ is substituted or unsubstituted arylalkyl. In some aspects, R′ is substituted or unsubstituted heteroalkyl. In some aspects, R′ is substituted or unsubstituted alkylheteroaryl. In some aspects, R′ is substituted or unsubstituted amine.

In some aspects, R″ is hydrogen. In some aspects, R″ is substituted or unsubstituted alkyl. In some aspects, R″ is substituted or unsubstituted alkenyl. In some aspects, R″ is substituted or unsubstituted ether. In some aspects, R″ is substituted or unsubstituted cycloalkyl. In some aspects, R″ is substituted or unsubstituted heterocyclyl. In some aspects, R″ is substituted or unsubstituted cycloalkenyl. In some aspects, R″ is substituted or unsubstituted aryl. In some aspects, R″ is substituted or unsubstituted heteroaryl. In some aspects, R″ is substituted or unsubstituted arylalkyl. In some aspects, R″ is substituted or unsubstituted heteroalkyl. In some aspects, R″ is substituted or unsubstituted alkylheteroaryl. In some aspects, R″ is substituted or unsubstituted amine.

In some aspects, R′″ is hydrogen. In some aspects, R′″ is substituted or unsubstituted alkyl. In some aspects, R′″ is substituted or unsubstituted alkenyl. In some aspects, R′″ is substituted or unsubstituted ether. In some aspects, R′″ is substituted or unsubstituted cycloalkyl. In some aspects, R′″ is substituted or unsubstituted heterocyclyl. In some aspects, R′″ is substituted or unsubstituted cycloalkenyl. In some aspects, R′″ is substituted or unsubstituted aryl. In some aspects, R′″ is substituted or unsubstituted heteroaryl. In some aspects, R′″ is substituted or unsubstituted arylalkyl. In some aspects, R′″ is substituted or unsubstituted heteroalkyl. In some aspects, R′″ is substituted or unsubstituted alkylheteroaryl. In some aspects, R′″ is substituted or unsubstituted amine.

In some aspects, R″″ is substituted or unsubstituted alkyl. In some aspects, R″″ is substituted or unsubstituted alkenyl. In some aspects, R″″ is substituted or unsubstituted ether. In some aspects, R″″ is substituted or unsubstituted cycloalkyl. In some aspects, R″″ is substituted or unsubstituted heterocyclyl. In some aspects, R″″ is substituted or unsubstituted cycloalkenyl. In some aspects, R″″ is substituted or unsubstituted aryl. In some aspects, R″″ is substituted or unsubstituted heteroaryl. In some aspects, R″″ is substituted or unsubstituted arylalkyl. In some aspects, R″″ is substituted or unsubstituted heteroalkyl. In some aspects, R″″ is substituted or unsubstituted alkylheteroaryl. In some aspects, R″″ is substituted or unsubstituted amine.

In some aspects, r is 1. In some aspects, r is 2. In some aspects, r is 3. In some aspects, r is 4. In some aspects, r is 5. In some aspects, r is 6.

In some aspects, Z is selected from the group consisting of capsaicin, bupivacaine, tetracaine, lidocaine, benzocaine, procaine, prilocaine, cinchocaine, ropivacaine, tetrodotoxin, saxitoxin, resiniferatoxin, botulinum toxin, a TRPV1 activator (e.g., gingerol, shogaol, zingerone, eugenol, N-arachidonoyl-dopamine, anandamide) and analogs thereof. In some aspects, Z is capsaicin. In some aspects, Z is bupivacaine. In some aspects, Z is tetracaine. In some aspects, Z is lidocaine. In some aspects, Z is benzocaine. In some aspects, Z is procaine. In some aspects, Z is prilocaine. In some aspects, Z is cinchocaine. In some aspects, Z is ropivacaine. In some aspects, Z is tetrodotoxin. In some aspects, Z is saxitoxin. In some aspects, Z is resiniferatoxin. In some aspects, Z is botulinum toxin. In some aspects, Z is a TRPV1 activator. In some such aspects, Z is gingerol. In some such aspects, Z is shogaol. In some such aspects, Z is zingerone. In some such aspects, Z is eugenol. In some such aspects, Z is N-arachidonoyl-dopamine. In some such aspects, Z is anandamide.

In some aspects, Z has the formula:

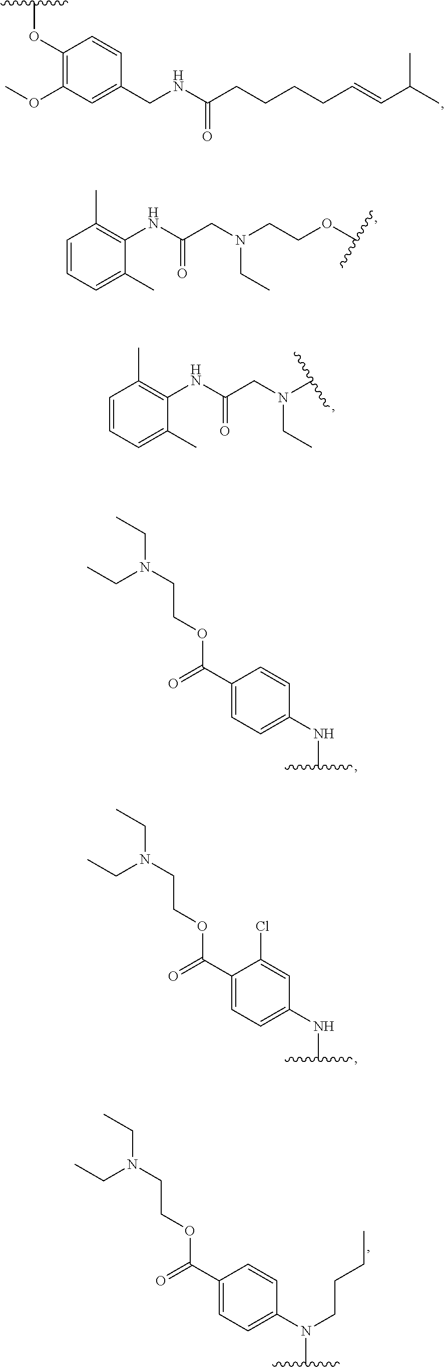

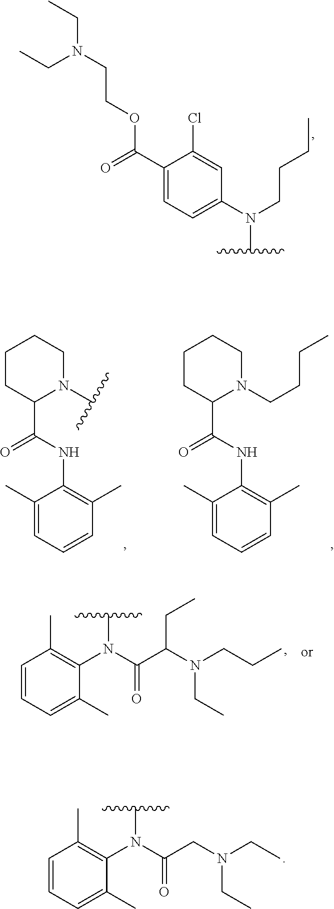 [SMILES: *N(CCCC)c1ccc(C(=O)OCCN(CC)CC)c(Cl)c1 ; *N1CCCCC1C(=O)Nc1c(C)cccc1C ; CCCCN1CCCCC1C(=O)Nc1c(C)cccc1C ; *N(C(=O)C(CC)N(CC)CCC)c1c(C)cccc1C ; *N(C(=O)CN(CC)CC)c1c(C)cccc1C]

In some aspects, the compound has the formula:

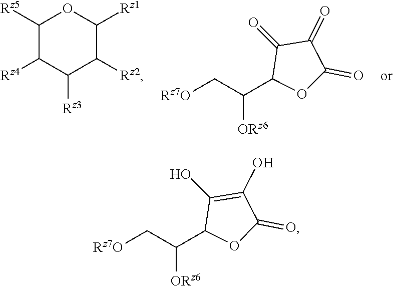

wherein: R^{Z1} is OH, OC₁₋₃alkyl, NH-L-Z, or O-L-Z; R^{z2} is H, OH, OR^{d}, OC₁₋₃alkyl, NHC(═O)CH₃, NHC(═O)CH₂-L-Z, NH-L-Z, O-L-Z, or -L-Z; R^{z3} is H, OH, OR^{d}, OC₁₋₃alkyl, NHC(═O)CH₃, NHC(═O)CH₂-L-Z, NH-L-Z, O-L-Z, or -L-Z; R^{z4} is H, OH, OR^{d}, OC₁₋₃alkyl, NHC(═O)CH₃, NHC(═O)CH₂-L-Z, NH-L-Z, O-L-Z, or -L-Z; R^{z}s is H, CH₃, CH₂OR^{d}, CH₂OH, CH₂OC₁₋₃alkyl, COOH, or -L-Z; R^{z6} is H or -L-Z; R^{z7} is H or -L-Z; R^{d} has the formula:

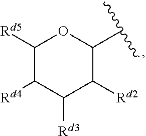

R^{d2} is H, OH, OC₁₋₃alkyl, NHC(═O)CH₃, NHC(═O)CH₂-L-Z, NH-L-Z, O-L-Z, or -L-Z; R^{d3} is H, OH, OC₁₋₃alkyl, NHC(═O)CH₃, NHC(═O)CH₂-L-Z, NH-L-Z, O-L-Z, or -L-Z; R^{d4} is H, OH, OC₁₋₃ alkyl, NHC(═O)CH₃, NHC(═O)CH₂-L-Z, NH-L-Z, O-L-Z, or -L-Z; R^{d5} is H, CH₃, CH₂OH, CH₂OC₁₋₃alkyl, COOH, or -L-Z; with the proviso that only one -L-Z group is present.

In some aspects, the compound has the formula:

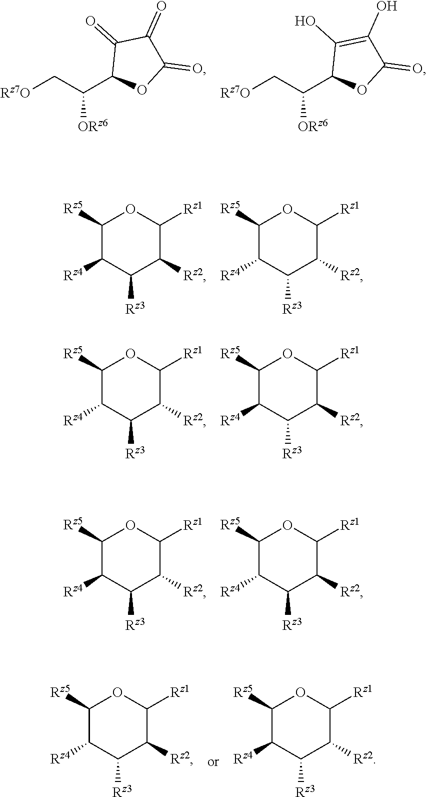

In some aspects, R^{d} has the formula:

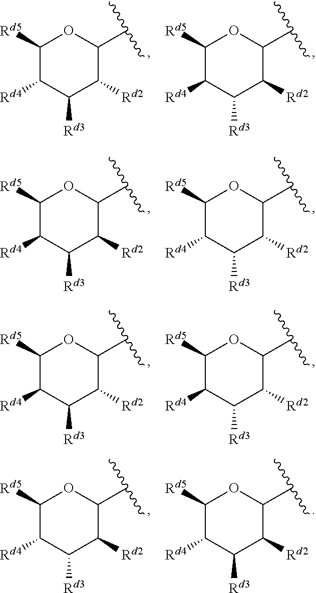

In some aspects, R^{Z1} is OH; R^{Z2} is OH; R^{Z3} is OH; R^{Z4} is OH; and R^{Z5} is CH₂O-L-Z.

In some aspects, R^{Z1} is OH; R^{Z2} is OH; R^{Z3} is OH; R^{Z4} is OH; and R^{Z5} is -L-Z.

In some aspects, R^{Z1} is OH; R^{Z2} is OH; R^{Z3} is OH; R^{Z4} is OH; R^{Z5} is -L-Z.

In some aspects, R^{Z7} is OH; and R^{Z6} is -L-Z.

In some aspects, R^{Z1} is OH; R^{Z2} is NH-L-Z; R^{Z3} is OH; R^{Z4} is OH; and R^{Z5} is CH₂OH.

In some aspects, R^{Z1} is OH; R^{Z2} is OH; R^{Z3} is OH; R^{Z4} is NH-L-Z; and R^{Z5} is CH₂OH.

In some aspects, -L-X is —C(═O)[CH₂].COOH or —C(═O)[CH₂]ₙCONH₂, wherein n is 1-6, preferably 2-4, more preferably 2-3.

In some aspects, R^{d2} is OH; R^{d3} is OH; R^{d4} is OH; and R^{d5} is CH₂OH.

In some aspects, the compound is selected from:

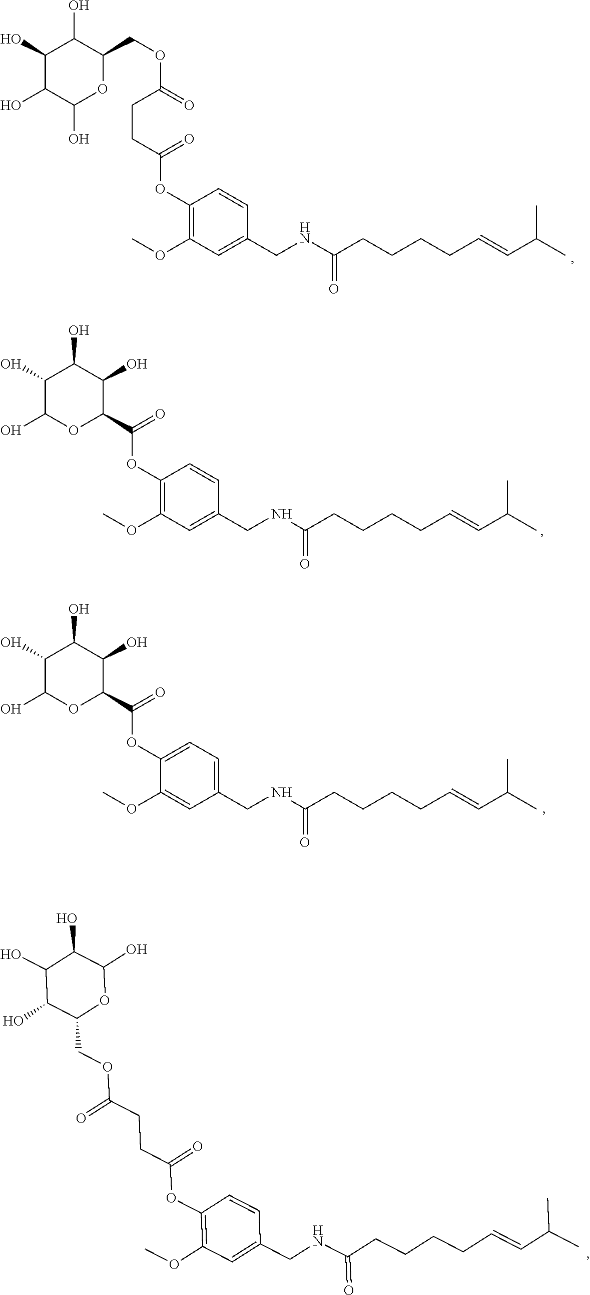 [SMILES: COc1cc(CNC(=O)CCCC/C=C/C(C)C)ccc1OC(=O)CCC(=O)OC[C@H]1OC(O)C(O)[C@@H](O)C1O ; COc1cc(CNC(=O)CCCC/C=C/C(C)C)ccc1OC(=O)[C@H]1OC(O)[C@H](O)[C@@H](O)[C@H]1O ; COc1cc(CNC(=O)CCCC/C=C/C(C)C)ccc1OC(=O)[C@H]1OC(O)[C@H](O)[C@@H](O)[C@H]1O ; COc1cc(CNC(=O)CCCC/C=C/C(C)C)ccc1OC(=O)CCC(=O)OC[C@H]1OC(O)[C@H](O)C(O)[C@H]1O]

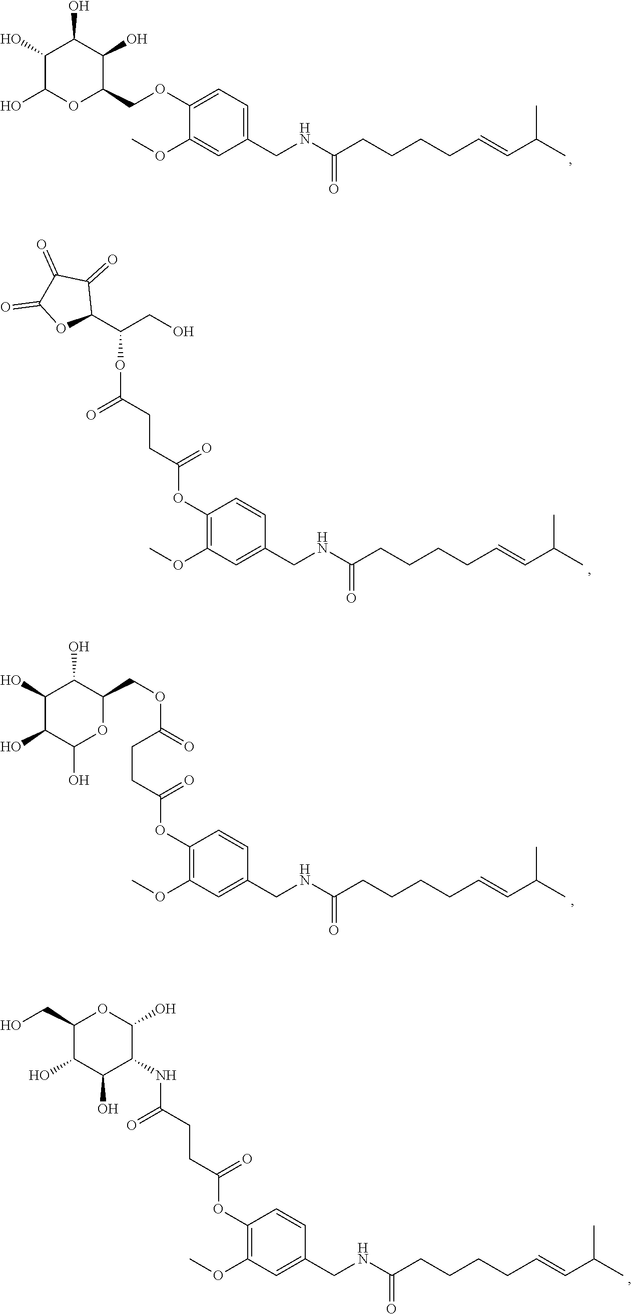 [SMILES: COc1cc(CNC(=O)CCCC/C=C/C(C)C)ccc1OC[C@H]1OC(O)[C@H](O)[C@@H](O)[C@H]1O ; COc1cc(CNC(=O)CCCC/C=C/C(C)C)ccc1OC(=O)CCC(=O)O[C@@H](CO)[C@H]1OC(=O)C(=O)C1=O ; COc1cc(CNC(=O)CCCC/C=C/C(C)C)ccc1OC(=O)CCC(=O)OC[C@H]1OC(O)[C@@H](O)[C@@H](O)[C@@H]1O ; COc1cc(CNC(=O)CCCC/C=C/C(C)C)ccc1OC(=O)CCC(=O)N[C@@H]1[C@@H](O)[C@H](O)[C@@H](CO)O[C@@H]1O]

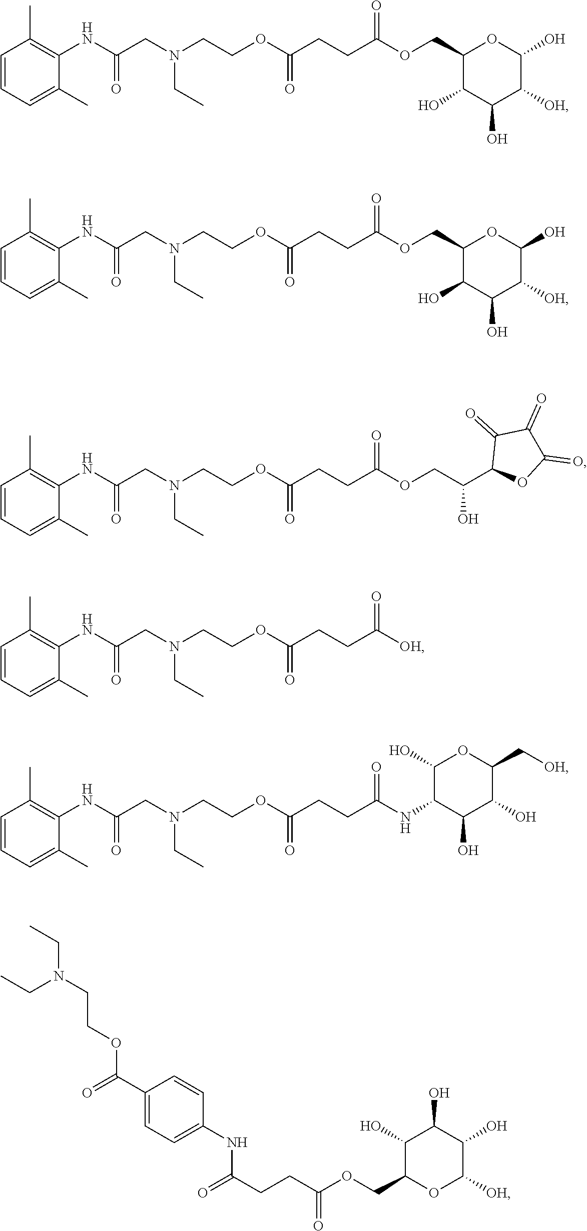 [SMILES: CCN(CCOC(=O)CCC(=O)OC[C@H]1O[C@H](O)[C@H](O)[C@@H](O)[C@@H]1O)CC(=O)Nc1c(C)cccc1C ; CCN(CCOC(=O)CCC(=O)OC[C@H]1O[C@@H](O)[C@H](O)[C@@H](O)[C@H]1O)CC(=O)Nc1c(C)cccc1C ; CCN(CCOC(=O)CCC(=O)OC[C@@H](O)[C@@H]1OC(=O)C(=O)C1=O)CC(=O)Nc1c(C)cccc1C ; CCN(CCOC(=O)CCC(=O)O)CC(=O)Nc1c(C)cccc1C ; CCN(CCOC(=O)CCC(=O)N[C@H]1[C@H](O)[C@@H](O)[C@H](CO)O[C@H]1O)CC(=O)Nc1c(C)cccc1C ; CCN(CC)CCOC(=O)c1ccc(NC(=O)CCC(=O)OC[C@@H]2O[C@@H](O)[C@@H](O)[C@H](O)[C@H]2O)cc1]

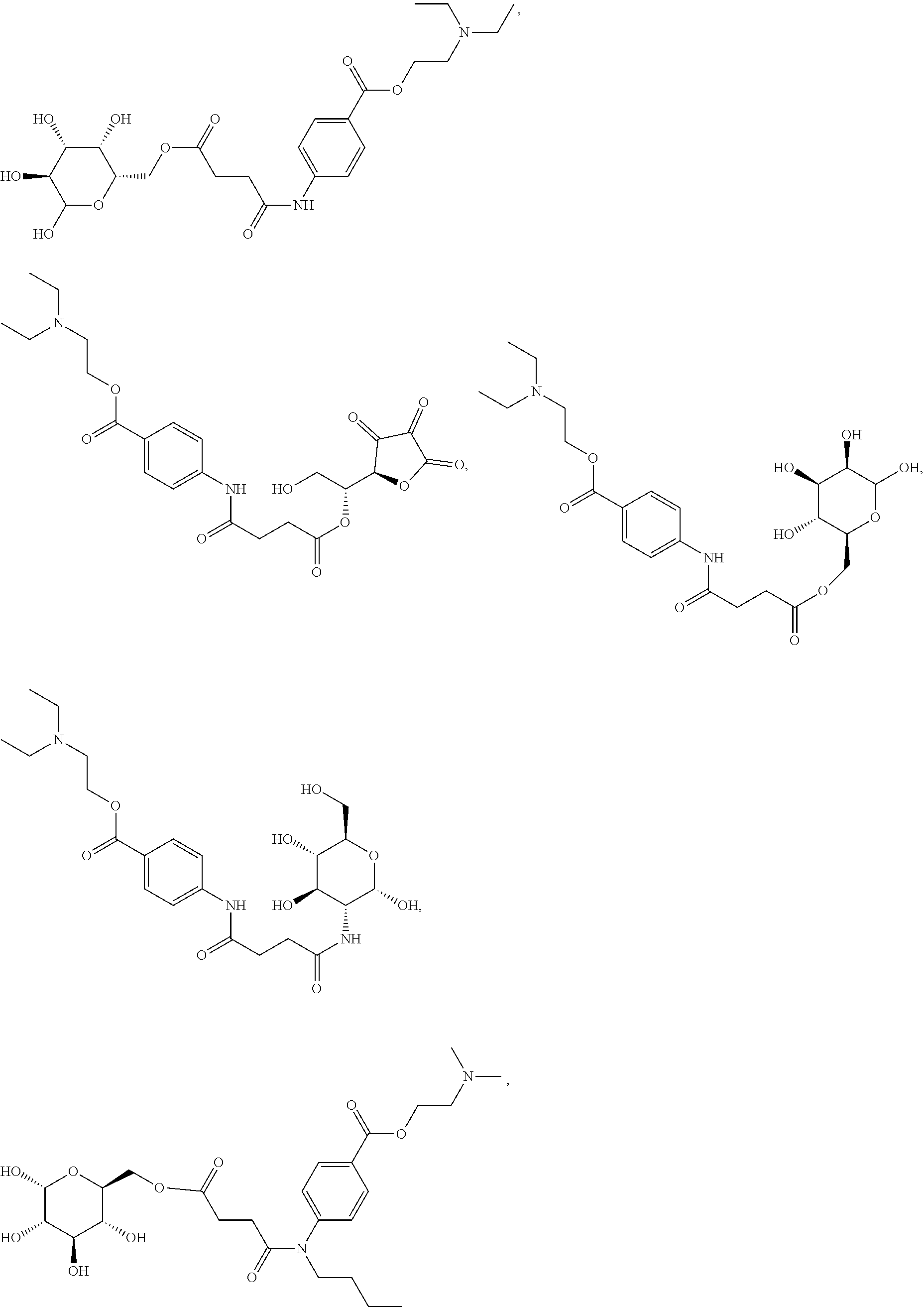

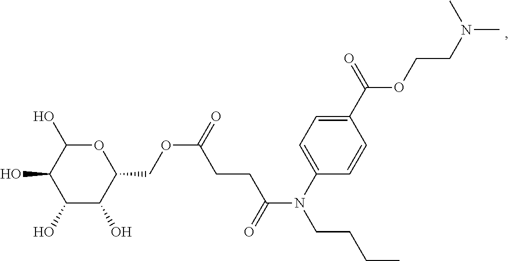 [SMILES: CCCCN(C(=O)CCC(=O)OC[C@H]1OC(O)[C@H](O)[C@@H](O)[C@H]1O)c1ccc(C(=O)OCCN(C)C)cc1]

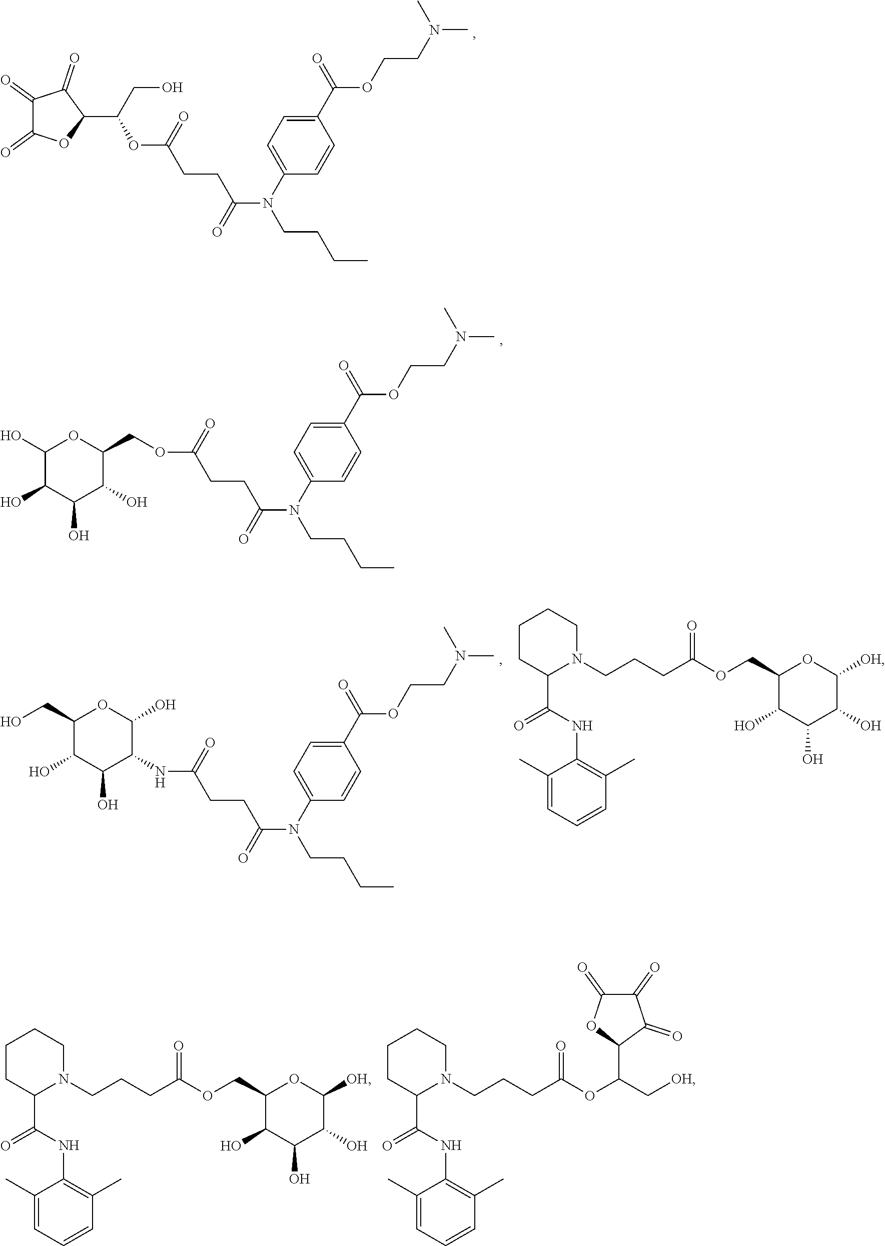

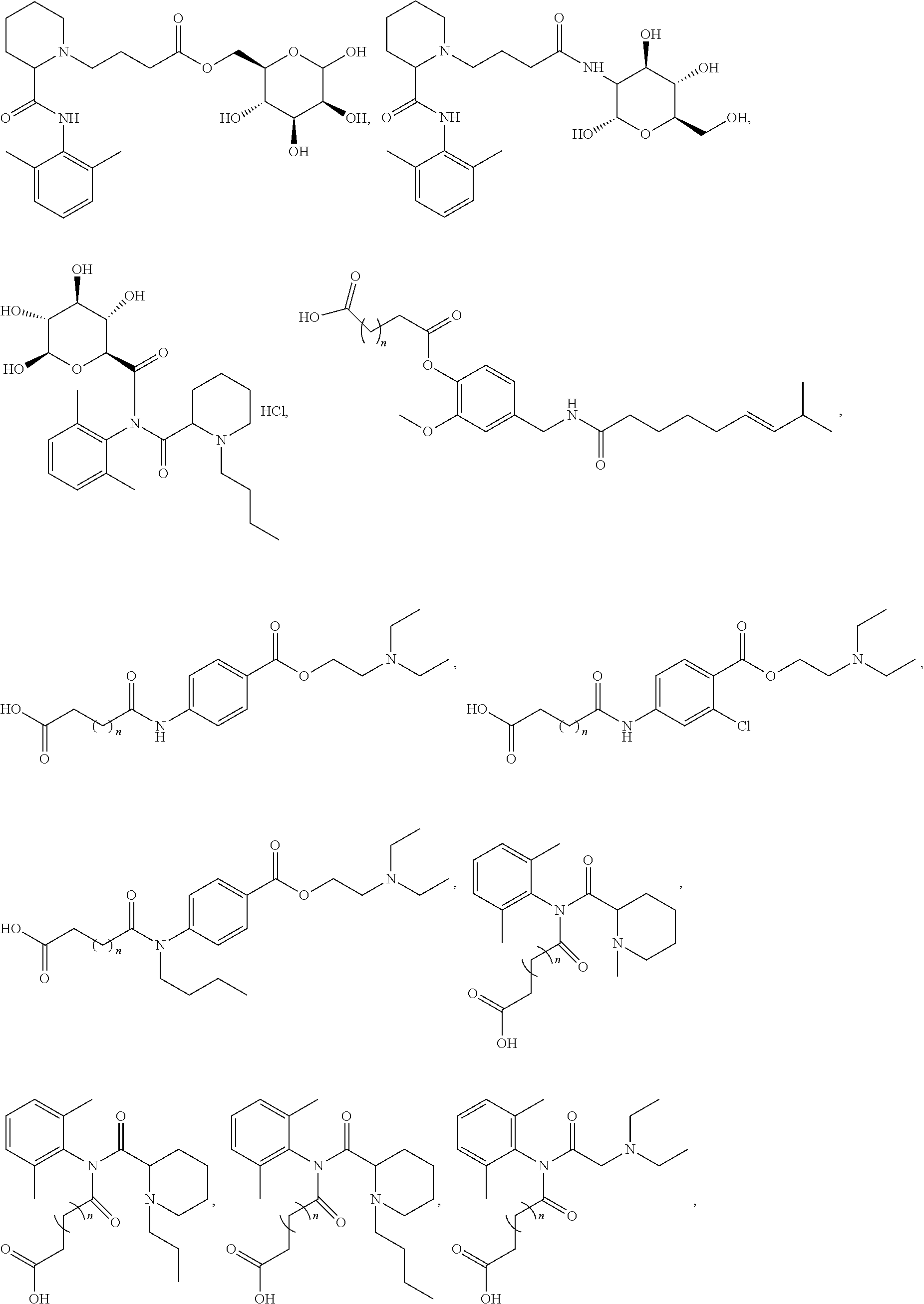

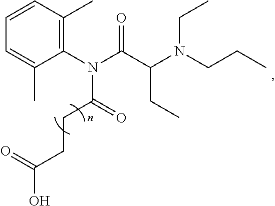

or pharmaceutically acceptable salts thereof.

In some aspects, the compound is

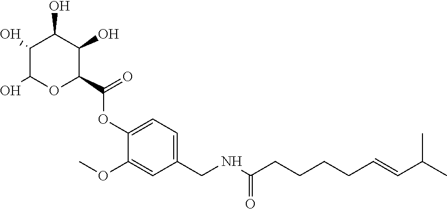 [SMILES: COc1cc(CNC(=O)CCCC/C=C/C(C)C)ccc1OC(=O)[C@H]1OC(O)[C@H](O)[C@@H](O)[C@H]1O]

or a pharmaceutically acceptable salt thereof.

In some aspects, the compound is

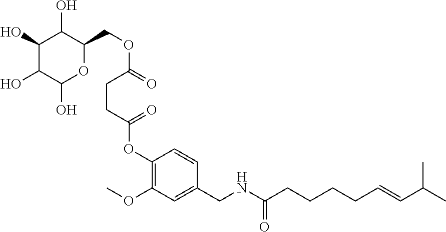 [SMILES: COc1cc(CNC(=O)CCCC/C=C/C(C)C)ccc1OC(=O)CCC(=O)OC[C@H]1OC(O)C(O)[C@@H](O)C1O]

or pharmaceutically acceptable salt thereof.

In some aspects, the compound has a high binding affinity to the GLUT1 transporter.

### EXAMPLES

### Example 1: Sugar Capsaicin Prodrugs for Prolonged Nociceptive-Selective Axon Blockade

### Prodrug Synthesis and Characterization

Since all sugars have similar molecular formulas (glucose, galactose, and fructose have the same chemical formula (C₆H₁₂O₆)) and the identical reactive primary hydroxyl (—CH₂OH) group, similar reactions can be applied to the synthesis of different sugar-capsaicin prodrugs. Sugar-capsaicin prodrugs can be synthesized linked with carbonate [38], amide [39], disulfide [40, 41], thioketal [42], ketal [43], and carbon chain [41], and chemical structure can be confirmed by NMR, FTIR, and LC-MS.

The chemical structure and composition of synthesized prodrugs can be analyzed using a proton nuclear magnetic resonance spectrometer (¹H NMR, Bruker 500) and a Fourier transform infrared spectrometer (FTIR, Alpha Bruker). Molecular weights can be ascertained using liquid chromatography-mass spectrometry (LC-MS). In addition, basic prodrug properties can be determined, including i) Water solubility: This is a critical parameter necessary to achieve the desired pharmacological response [33]. Poor water solubility is a common reason for low drug bioavailability. Moreover, water solubility is a key factor that guides injectable prodrug formulation development; ii) Differential environment prodrug chemical hydrolysis: Prodrug stability can be studied in a pro-inflammatory pain microenvironment. Specifically, the effect of pH on prodrug stability can be determined because chronic pain is commonly associated with an acidic tissue environment [30, 34] and ester/ketal/carbonate bonds are susceptible to acidic hydrolysis. Since inflamed tissues may overexpress enzymes and ester/amide/carbonate bonds are susceptible to enzymatic hydrolysis, prodrug degradation kinetics can be measured in PBS containing rat brain homogenate or rat liver homogenate, both of which contain esterase enzymes. Inflamed tissues are typically associated with elevated ROS levels that support progressive tissue injury [35, 36]. In addition, elevated ROS level have commonly been implicated in neurodegenerative diseases [37]. As a result, prodrug degradation kinetics can also be measured in PBS solutions containing ROS; iii) Cytotoxicity: Validated cytotoxicity assays can be performed to determine prodrug myotoxicity and neurotoxicity potential.

### Water Solubility and Partition Coefficients of Prodrug in Octanol/Water:

Water solubility can be determined by sonicating excess prodrug in water for 15 min at room temperature, followed by centrifugation at 10,000 g for 2 min. The supernatant can be analyzed by HPLC and its partition coefficient (octanol/water) determined by vortexing the prodrug and octanol/water mixture for 10 min. Prodrug concentrations in the octanol and water phases can be ascertained by HPLC. Prodrug partition coefficient is calculated as follows: [c]_{octanol}/[c]_{water}.

### Fabrication of Injectable Prodrugs:

If the drug concentration to be tested falls below prodrug water solubility, an injectable formulation can be developed by dissolving the sugar-capsaicin prodrug directly in a 1×PBS buffer (pH 7.4). If the drug concentration exceeds prodrug water solubility, an injectable formulation can be developed with Tween 80 as a surfactant to generate a uniform prodrug suspension [45].

### Chemical Hydrolysis of Prodrugs:

The rate of prodrug chemical hydrolysis in 1×PBS at different pH, enzyme concentrations, and oxidative stress levels can be quantified. Specifically, a predefined injectable prodrug amount can be added to 10 mL of PBS at pH=6.0, 7.0, 7.4 and 7.8, or PBS with rat brain homogenate (20% v/v) [46], or PBS with H₂O₂(30% w/w) and Fenton (2 mg/mL) [42]. All solutions can be placed in a thermostatically controlled water bath at 37° C. At pre-defined intervals, samples can be aliquoted and analyzed for remaining prodrug by HPLC and LC-MS. Pseudo-first-order half-time (t_{1/2}) for the prodrug hydrolysis can be calculated from the slope of the linear portion of the remaining prodrug plotted logarithm against time.

### Cell Viability:

C2C12 mouse myoblasts, PC12 rat adrenal gland pheochromocytoma cells (a cell line frequently used in neurotoxicity studies [47, 48]), and rat DRG neurons [49] can be used to evaluate prodrug in vitro muscle cell and neuronal cell cytotoxicity, respectively. Briefly, cells can be incubated (1×10⁴ per well) with different prodrug concentrations (0.01, 0.1, 0.5, 1 mg/mL) for 24 hours in an incubator at 37° C. in 5% CO₂. After incubation, cells can be washed 5 times with warmed 1×PBS to remove any remaining prodrug, and cell viability can be determined by the 3-(4,5-dimethylthiazol-2-yl)-2,5-diphenyltetrazolium bromide (MTT) assay and live/dead assay [50]. Pure capsaicin can be administered as a positive control in such experiments.

### Synthesis and Characterization of a Galactose-Capsaicin Prodrug:

A galactose-capsaicin prodrug was synthesized by linking capsaicin to galactose through an ester bond (FIG. 3). The chemical structure of the synthesized prodrug was confirmed by proton nuclear magnetic resonance spectroscopy (¹H NMR) and matrix-assisted laser desorption/ionization-time of flight (MALDI-TOF) mass spectrometry (MS). Based on the integrated intensity analysis of ¹H NMR spectrum, a 98% prodrug purity was ascertained.

Selecting Prodrugs with High In Vitro BNB/Perineurium Permeability

Peripheral nerves are metabolically active. Glucose, a polar hydrophilic molecule, can gain access into peripheral nerve endoneurium from the blood circulation or interstitial fluid across restrictive cellular barriers. Perineurial cells and endothelial cells that form the perineurium and blood-nerve barrier (BNB) express multiple glucose transporters (GLUTs), with glucose transporter protein type 1 (GLUT1) being the most prevalent [17, 18]. GLUT1 facilitates the transport of glucose and a variety of other sugar molecules across these PNBs by carrier-mediated mechanisms [19-22].

GLUT1 is highly expressed by endoneurial microvascular endothelial cells that form the human BNB and perineurium in vitro and in situ [55-57], as well as microvascular endothelial cells that form the blood-brain barrier (BBB). GLUT1 facilitates glucose transport across the BBB into the brain parenchyma [58, 59]. In vitro solute permeability assays provide a high throughput avenue to study molecular permeability kinetics across endothelial and epithelial barrier systems.

Static transwell insert solute permeability studies can be performed using a simian virus long T antigen immortalized human endoneurial endothelial cell (THEndEC) line that retains essential characteristics of the human BNB [55-57]. Briefly, 80,000 THEndECs can be seeded and grown to confluence on glutaraldehyde-crosslinked, rat tail collagen-coated 6.5 mm diameter Coring polyester membrane Transwell© inserts (0.33 cm² surface area, 0.4 m pore size) placed in 24-well tissue culture plates for 5 days in growth medium, and serum withdrawal can be performed for 2 days to inhibit growth prior to the permeability assays. Transendothelial electrical resistance (TEER) can be routinely monitored using a voltohmmeter (EVOM, World Precision Instruments) in concurrently plated inserts to ensure maximum expected TEER prior to permeability assays. Collagen-coated Transwell inserts without cultured THEndECs can be used as permeability assay controls. On day 7 after plating, Transwell inserts can be carefully transfered into freshly prepared 24 well plates containing 600 μL of 1% bovine serum albumin (BSA) in RPMI-1640, and 100 μL of predefined concentrations of selected capsaicin-linked prodrugs or capsaicin in 1% BSA in RPMI-1640 can be added into each insert in triplicate. The solute permeability assays can be performed in a humidified incubator at 37° C. in 5% CO₂ for 6 hours. At predefined periods, 24 well plates can be removed from the incubator, and the solute can be carefully removed and aspirated from the Transwell insert into Eppendorf tubes while leaving solute in the 24-plate wells. Solutes in the inserts and wells can be quantified by HPLC and LC-MS, and solute permeability can be determined as a percentage of the input.

To determine in vitro perineurium permeability, similar experimental methods can be used, with the exception of using a commercially available primary human perineurial cell line (e.g. ScienCell catalog #1710) instead of the THEndEC cell line.

### GLUT1 Transporter-Mediated Glucose-UDP-Fluorescein Conjugate Permeability Across the BBB In Vitro:

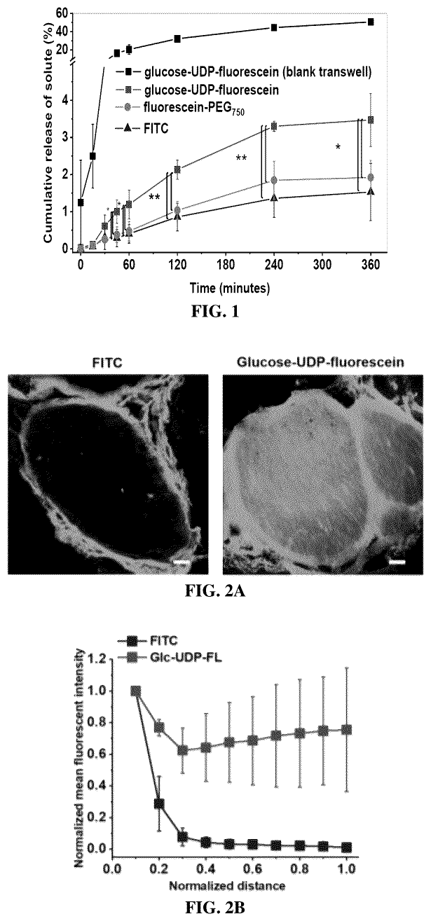

Brain microvascular endothelial cells (BMVECs) are the restrictive tight junction forming component of the BBB and they express the GLUT1 transporter [24]. In order to investigate whether sugar molecules enhance prodrug BBB permeability via GLUT-1, an in vitro BBB Transwell model was employed. The time-dependent permeability of fluorescein isothiocyanate (FITC), fluorescein polyethylene glycol derivative (fluorescein-PEG₇₅₀), and fluorescein uridine-5′-diphospho-1-alpha-D-glucose derivative (glucose-UDP-fluorescein) was quantified through transwell insert with confluent cultured BMVECs after 10 days. Fluorescein-PEG₇₅₀ was chosen as a negative control for glucose-UDP-fluorescein, as it does not contain the sugar molecule but has a similar molecular weight and hydrophilicity. As shown in FIG. 1, the glucose-UDP-fluorescein conjugate has a significant mean higher solute permeability at the in vitro BBB compared to FITC and fluorescein-PEG₇₅₀. This finding implies that the glucose moiety drives fluorescein molecule transport across the in vitro BBB, possibly via GLUT1-mediated carrier transport.

Selecting Prodrugs with High Binding Affinity to the GLUT1 Transporter

Although it is widely accepted that GLUT1 actively transports glucose-based prodrugs from blood circulation across the BBB into the brain [22, 25, 26], it remains unclear whether GLUT1 actively transports peripherally injected glucose-based prodrugs across PNBs into peripheral nerve endoneurium. To determine binding affinity, increasing prodrug concentrations can be co-perfused with [¹⁴C]D-glucose (0.2 μCi/mL) into rat brains for 30 seconds. The permeability-surface area product (PS) of [¹⁴C]D-glucose can be determined after the perfusion. The prodrug concentration dependent inhibition of [¹⁴C]D-glucose uptake can be evaluated, and its half maximal inhibitory concentration (IC₅₀) values can be deduced to compare GLUT1 binding affinities.

Selecting Prodrugs with High In Vivo Peripheral Nerve Penetration

Local anesthetics are administered around peripheral nerves and have to pass through tight junction forming PNBs, particularly the perineurium, into the endoneurium to inhibit axonal signal transduction. Previous studies show that perineurial cells highly express GLUT1 [17][60, 61]. This experiment can be used to evaluate prodrug peripheral nerve permeability to determine whether the sugar moiety enhances prodrug peripheral nerve permeability in vivo.

Under isoflurane-oxygen anesthesia, 0.5 mg of prodrugs in 0.3 mL 1×PBS can be injected in the subcutaneous tissue above the left sciatic nerve with a 23 G×¾″ needle. The needle can be introduced postero-medial to the greater trochanter, pointing in the anteromedial direction, and upon contact with bone the needle can be withdrawn slightly and the formulation can be injected above the sciatic nerve.

Four hours after prodrug sciatic nerve injections, carbon dioxide (CO₂) asphyxiation can be performed to euthanize rats and harvest the sciatic nerves from both sides (the sciatic nerve on the right side of the same rat can be used as the blank control). Sciatic nerve endoneurial prodrugs and capsaicin (e.g. the prodrug hydrolyzed product) can be extracted as previously reported [62, 63]. Prodrug and capsaicin amount can be quantified by HPLC and LC-MS, respectively. Endoneurial capsaicin quantities can be converted into the corresponding prodrug quantity. The total amount of sciatic nerve endoneurial prodrug is the sum of the directly measured prodrug quantity and the calculated prodrug quantity derived from endoneurial capsaicin. Prodrug peripheral nerve permeability can be calculated as follows: solute permeability (%)=(amount of endoneurial prodrug/amount of prodrug administered)×100%. 0.5 mg of capsaicin in 0.3 mL 1×PBS can be injected as a control and its permeability calculated for comparative analyses. For each group, an equal number of male and female rats can be used for such a test.

### GLUT1 Transporter-Mediated Glucose-UDP-Fluorescein Conjugate Endoneurial Permeability In Vivo:

A rat sciatic nerve in vivo permeability model was utilized to study GLUT1 carrier-mediated prodrug permeability in peripheral nerves. Adult (>70 day old) male Sprague-Dawley rats weighing 250-350 g were perineurally injected with 0.3 mL of Phosphate-Buffered Saline (PBS, 1×pH 7.4) solution containing FITC or glucose-UDP-fluorescein at equal fluorescent intensities above the sciatic nerves. Four hours after injection, the rats were euthanized, and the sciatic nerves and surrounding tissues were harvested, frozen, sectioned, and visualized using a confocal microscope. As shown in FIG. 2A, in rats injected with FITC, fluorescence was observed in the surrounding epineurium. This observation indicates that FITC was unable to transverse the perineurium into the sciatic nerve endoneurium. It was observed that FITC accumulated in the hamstring muscles and associated adipose tissue around the sciatic nerve. In contrast, a uniform fluorescence distribution was observed within and outside the sciatic nerve endoneurium in rats injected with glucose-UDP-fluorescein. There was no accumulation of glucose-UDP-fluorescein in the surrounding epineurium. A quantitative analysis showed that the fluorescence signal persisted deeper into the nerves of rats injected with glucose-UDP-fluorescein (FIG. 2B). The results show that glucose-UDP-fluorescein can cross the restrictive perineurium into the endoneurium and persist within sciatic nerves in vivo.

### Sciatic Nerve Blockade in Normal Rats

To characterize sugar-capsaicin prodrug effect on sensory and motor function, duration of action, and safety in normal adult rats, selected prodrugs can be perineurally injected in the deep subcutaneous tissues over the left sciatic nerves in adult Sprague-Dawley rats (males: 250-350 g/females: 225-325 g). At predetermined time intervals, a battery of validated neurobehavioral tests can be performed to evaluate sensory and motor functions in each rat.

Each rat can be evaluated for sensory and motor functional deficits following prodrug administration. These functional deficits indicate anesthesia, weakness or both involving the left hind limb below the knee. The degree of nociceptive axon and motor axon blockade can be ascertained using the thermal nociceptive/Touch-Test sensory tests, and weight-bearing test, respectively.

### Sciatic Nerve Injection:

Under isoflurane-oxygen anesthesia, local anesthetic can be injected with a 23 G×¾″ needle posteromedial to the greater trochanter, pointing in an anteromedial direction in the deep subcutaneous tissue over the left sciatic nerve [50, 74]. Once bone is contacted, the needle can be slightly withdrawn, and 0.3 mL of the test solution containing an increased dose of prodrugs can be injected. The control rats can receive 0.3 mL test solution containing saline or capsaicin.

### Intravenous Injection:

Under isoflurane-oxygen anesthesia, 0.3 mL of 1×PBS solution containing an increased dose of prodrugs/capsaicin can be injected via the tail vein using a 23 G×¾″ needle.

### Sensory Function Test:

Nociceptive axon function can be evaluated via the thermal nociceptive and Touch-Test sensory tests. The thermal nociceptive test can be performed using the modified hotplate testing [50]. In brief, hind paws can be sequentially exposed (left then right) to a 56° C. hot plate (Model 39D Hot Plate Analgesia Meter, IITC Inc., Woodland Hills, CA), and the time to paw withdrawal (thermal withdrawal latency) can be measured using a digital stopwatch. A thermal paw withdrawal latency of 2 seconds indicates no nerve blockade (baseline). The maximum thermal paw withdrawal latency can be capped at 12 seconds in order to prevent tissue injury. Sciatic nerve nociceptive axon blockade is defined as a thermal paw withdrawal latency above 7 seconds [50]. These assessments can be performed three times in each rat at the predetermined time points after a rest period, and the mean withdrawal latency can be used for subsequent comparative analyses. The Touch-Test sensory test can be performed at predetermined intervals after injection by stimulating the hindfoot pad with a Touch-Test sensory evaluator (monofilament with target force of 180 g) and noting the rat's vocal or motor response (foot withdrawal) [75-78]. Complete nociceptive axon blockade (also known as 100% maximum peak effect (MPE)) can be defined as the absence of vocalizations or paw withdrawal after five trials. Duration of block can be defined as the total time >50% MPE. Before injections, each rat's baseline tactile paw withdrawal (i.e. the force threshold to which the rat will respond) can be determined using monofilaments with gradually increased target forces (26, 60, 100, 180 g).

### Motor Function Test:

Sciatic nerve motor axon blockade can be evaluated following prodrug/capsaicin injection using a weight-bearing test [50]. In brief, each rat can be placed on a digital balance one hind paw at a time and allowed to bear its own weight. The maximum weight that the rat could bear without the ankle touching the balance can be recorded, and motor block can be considered achieved when the motor strength is less than half maximal. Measurements can be repeated three times at each time point and the median can be used for further data analysis.

### Duration of Nerve Blockade:

Blinded neurobehavioral tests can be performed every 30 minutes for the first 2 hours, then hourly for the next 4 hours and every 6 hours for the next 24 hours after injection, and then twice a day until nerve blockade resolves. Nociceptive axon blockade duration can be defined as the time required for thermal latency to return to 7 seconds, while motor axon blockade duration can be defined as the time required for weight bearing to return to the midpoint between normal function and maximal blockade.

### Sciatic Nerve Blockade in Rat Models of Inflammatory Pain and Traumatic Nerve Injury

Prodrug duration of action and safety can be evaluated in two animal models of pain. These studies can provide essential proof-of-principle data to guide future early phase clinical trials in chronic pain syndrome patients. Persistent paw inflammation can be studied as a model of chronic inflammatory pain [79] and spared sciatic nerve injury as a model of traumatic nerve injury [80]. Both models induce injury-induced hyperalgesia that persists for weeks to allow determination of nociceptive axon blockade duration following prodrug administration.

Cohorts of adult Sprague-Dawley male and female rats weighing 225-350 g can be randomly assigned to receive deep subcutaneous prodrug or capsaicin or saline injection (as described above) after unilateral intraplantar Complete Freund's adjuvant (CFA) or spared sciatic nerve injury in the same hind limb 24 hours or 7 days afterwards, respectively. Validated neurobehavioral assessments can be performed to evaluate prodrug efficacy on reflexive nociception, and safety, as based on signs of local and systemic toxicity, compared to capsaicin.

### Rodent Chronic Nociception Models: Induction of CFA-Induced Inflammatory Pain:

Under brief isoflurane-oxygen anesthesia, 150 μL CFA can be subcutaneously injected into the plantar aspect of the left hind paw, using a 1 mL syringe with a 26 G beveled needle.

### Induction of Traumatic Nerve Injury:

Under isoflurane anesthesia, the distal left sciatic nerve can be exposed using sterile techniques, the common peroneal and tibial nerves litigated without touching the sural nerve, and the subcutaneous tissue and skin stitched back with nylon suture. Buprenorphine (0.03 mg/kg) can be given by subcutaneous injection in the immediate post-procedure phase up to 72 hours.

### Prodrug Treatment:

0.3 mL of different concentrations of selected prodrugs can be injected 24 hours after inducing paw inflammation or 7 days after spared sciatic nerve injury in the deep subcutaneous tissue over the left sciatic nerve with a 23 G×¾″ needle under brief isoflurane-oxygen anesthesia. An equal volume of equivalent capsaicin concentration (and normal saline) can be administered to age- and sex-matched controls, and sex can be evaluated as a biological variable in such experiments.

### Neurobehavioral Reflexive Nociception Assessment:

Blinded reflexive neurobehavioral tests can be performed in each experimental rat at predefined time intervals to determine prodrug analgesic efficacy relative to controls. These validated published assessments include thermal hyperalgesia (Hargreaves test), mechanical hypersensitivity (simplified up-down method) and cold allodynia (cold plantar assay) [81-83].

### Thermal Hyperalgesia:

Paw withdrawal latency to an infrared beam applied to the hind paw plantar surface through a Plexiglass platform can be measured using a modified Hargreaves box in all experimental rats. This system can automatically measure paw withdrawal latency following withdrawal from the infrared beam. Testing can be terminated if the rat does not withdrawal its hind paw by 20 seconds to prevent tissue injury, and a score of “20” can be assigned. Measurements can be repeated three times at each time point and the median can be used for further data analysis. Absolute (in seconds) and normalized (i.e. % of initial) withdrawal latencies can be plotted relative to time after drug administration (effect-time curves) to determine prodrug analgesic efficacy compared to controls.

### Mechanical Hypersensitivity:

Rats can be placed in plastic, wire mesh-bottomed cage with divided compartments for each rat. After 30 minutes of accommodation to allow rats to habituate to this environment, graduated von Frey filaments can be applied to the hind paw plantar surface, and the 50% paw withdrawal mechanical threshold can be determined fusing validated up-down paradigms.

### Cold Allodynia:

In the same test environment used for mechanical hypersensitivity assessment, a small pellet of dry ice can be applied to the hind paw plantar surface and withdrawal latency measured with a digital stopwatch. Absolute (in seconds) and normalized (i.e. % of initial) withdrawal latencies can be plotted relative to time after drug administration (effect-time curves) to determine prodrug analgesic efficacy compared to controls.

### Motor Effect:

The effect of the motor block can be assessed in the paw inflammation model and the mononeuropathy model. Hind paw grip strength can be assessed by measuring the force required to distract either hind paw of the rat from grasping a wire mesh grid. The rats can be allowed grasp with one paw a portion of a fixed grid attached to a strain gauge. The animals can be gradually removed from the grid. The sample and hold tension where the animal is disconnected from the grid can be assessed.

Thermal Nociception Blockade but not Motor Blockade with Galactose-Capsaicin Prodrugs

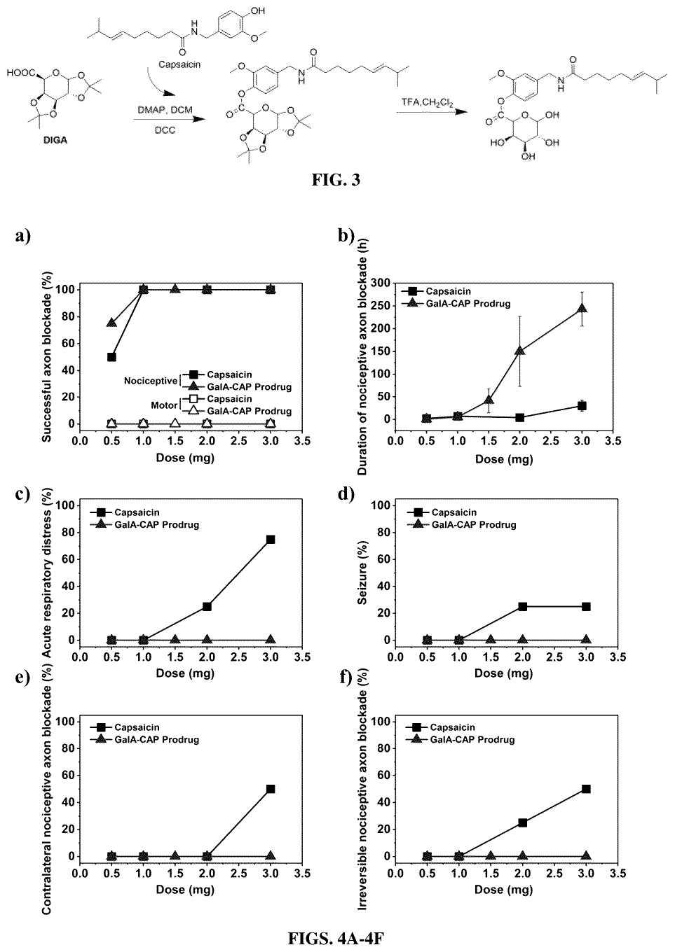

The in vivo efficacy and safety of the synthesized galactose-capsaicin prodrug and pure capsaicin was tested in blocking rat sciatic nerves. Adult male Sprague-Dawley rats weighing 250-350 g were injected with increasing doses of galactose-capsaicin prodrug or pure capsaicin (dissolved in 0.3 mL of 1×PBS buffer) peripherally at the left sciatic nerve. Neurobehavioral tests were performed to determine the duration of functional deficits, including nociceptive (measure paw withdrawal latency using a hotplate test) and motor axon blockade (measure the maximum weight a rat could bear using a weight-bearing test), in both hindpaws. Cohorts of rats that received injections of either the prodrug or pure capsaicin showed evidence of successful nociceptive axon blockade (i.e. thermal nociception blockade) in a dose-dependent manner with differences in duration of nerve block and side effect frequencies (FIGS. 4A-4F). Specifically, both groups achieved a 100% successful analgesic block (i.e. all rats tested showed thermal paw withdrawal latencies >7 seconds) at injection doses above 1.0 mg (FIG. 4A). In addition, the injection did not produce any motor deficits (i.e., from motor fascicular blockade) in the injected hind limb. A reversible nociceptive axon blockade was successfully induced following a single prodrug injection of 2 and 3 mg lasting for 150.0±76.8 and 234±36 hours, respectively (FIG. 4B). Importantly, the injection did not cause any capsaicin-related side effects, such as spontaneous pain behavior (e.g. flinching, licking), contralateral nociceptive axon blockade in the uninjected right hind limb, irreversible nociceptive axon blockade, seizure, or acute respiratory distress. In contrast, injection of 2 and 3 mg of pure capsaicin produced a nociceptive axon block lasting for 4±2 and 30±12 hours, respectively. It was observed that all rats injected with 3 mg of pure capsaicin exhibited at least one systemic adverse effect. Specifically, 75% developed acute respiratory distress, 25% experienced a seizure, 50% developed contralateral nociceptive axon block, and 50% developed irreversible nociceptive axon block in the injected hind limb, indicating capsaicin-induced nerve damage.

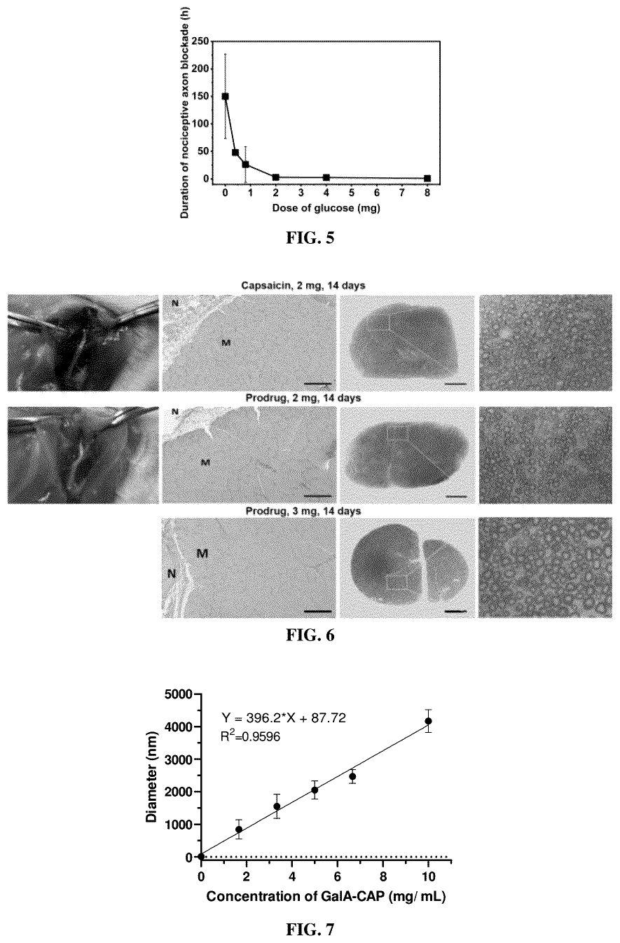

To confirm that the enhanced prodrug efficacy was due to the GLUT1-mediated conjugated galactose transport across the sciatic nerve perineurium, 2 mg of the prodrug was co-administered with increasing amounts of pure glucose. As the dose of glucose increased, a significant decrease was observed in nociceptive axon block duration (FIG. 5). This indicates that prodrug uptake was competitively inhibited by glucose, supporting the notion that conjugated galactose facilitates sciatic nerve prodrug permeability through GLUT1-mediated transport.

In order to study the local effects of capsaicin and prodrug injections, treated rats were euthanized 14 days after injection. The sciatic nerves and their surrounding tissues were harvested, sectioned, and stained for histologic evaluation. Hamstring muscles were processed and stained with hematoxylin-eosin (H&E). Epon-embedded semi-thin sciatic nerve sections were generated and stained with toluidine blue (goldstandard for peripheral nerve morphology). It was observed that the surrounding tissues in rats injected with 2-3 mg of capsaicin appeared reddish and swollen. Demyelinated axons were also observed in toluidine blue stained sections (FIG. 6). In contrast, the surrounding tissues in rats injected with 2-3 mg of prodrug, did not appear edematous or discolored, and there were no obvious signs of tissue injury. Microscopic examination did not reveal significant myotoxicity or inflammation. Nerve sections stained with toluidine blue were normal in appearance without evidence of demyelination or axonal degeneration in any of the treated rats.

### Drug Toxicity

Systemic Toxicity Associated with Capsaicin:

There are two principal clinical measures that indicate capsaicin-induced systemic toxicity [66, 67]: i) sciatic nerve blockade in the un-injected (contralateral) limb suggests systemic drug distribution, and ii) clinical signs of distress. Neurobehavioral tests can be used to evaluate nociceptive axon and motor axon blockade in the right sciatic nerve. Rats can be closely observed for signs of distress, which include seizures, excessive salivation, staggering gait, irreversible nerve block, respiratory distress, and death [68]. Rats that show agonal breathing or apneic spells can be immediately euthanized.

Increasing doses of prodrugs or capsaicin can be administered, and neurobehavioral assessments can be performed in different groups of rats until signs of systemic toxicity are observed. From these series of experiments, duration of nociceptive axon and motor axon blockade can be determined for each prodrug at different concentrations. Toxicity can be quantified as the dose per kg that caused the adverse effect in 50% of rats (i.e. the median effective dose (ED₅₀)) for each of the toxicological endpoints.

Intravenous injection of a compound can generally be assumed to be a bolus injection (i.e., it is delivered within a few seconds) [69, 70]. Since inadvertent intravascular injection can cause systemic anesthetic toxicity, safety of intravenous prodrugs/capsaicin administration can be evaluated.

### Local Drug Toxicity Studies:

Local tissue toxicity has been a major obstacle to developing long-duration local anesthetics [84, 85]. Intrinsic cellular toxicity and inflammation related to the drug-delivery vehicle could cause local skin, muscle, and nerve injury. High capsaicin doses can cause degeneration of DRG from small unmyelinated peripheral axons [86], and this axonal or DRG damage can lead to irreversible sciatic nerve block [87]. Local tissue reaction to prodrug administration compared to capsaicin can be studied.

### Histology:

Rats treated with prodrugs or capsaicin can be euthanized by carbon dioxide asphyxiation on day 4 and 14 after drug administration. Sciatic nerves can be dissected from just above the greater trochanter to its trifurcation in the popliteal fossa and immediately placed in fixative as described below. The long head of the biceps femoris muscle can be dissected and placed in a mold filled with Optimum Cutting Temperature (OCT) for H&E-stained cryostat sections.

### Inflammation [95]:

Blinded semi-quantitative histology scores can be generated to determine muscle inflammation severity as follows: 0. normal; 1. perifascicular inflammation; 2. partial intrafascicular inflammation; 3. hemifascicular inflammation; 4. holofascicular inflammation.

### Myotoxicity [96, 97]:

Blinded semi-quantitative scores can be generated to determine muscle injury based on myofiber nuclear internalization and degeneration. Nuclear internalization can be characterized by normal appearing myofibers with internalized nuclei or pyknotic nuclear clumps, whole degeneration can be characterized by myofibers with altered intramyofibrillar structure such as pale or hyalinized eosinophilic sarcolemma. Myotoxicity can be scored based on % myofibers with internalized nuclei or pkynotic nuclear clumps and the % degenerating myofibers.

To evaluate local neurotoxicity, axonal degeneration and demyelination can be evaluated in harvested sciatic nerve sections close to the site of drug administration. Sciatic nerves can be fixed with Karnovsky's K11 Solution (2.5% GluTaraldehyde, 2.0% paraformaldehyde, 0.025% calcium chloride in 0.1 M cacodylate buffer, pH 7.4) and postfixed with 1% osmium tetroxide prior to generating epon-resin embedded blocks. 0.5 m thick sections can be generated, stained with toluidine blue and visualized via light microscopy at high magnification to identify both myelinated and unmyelinated axons. Axonal degeneration can be quantified as the % of axons undergoing Wallerian degeneration and demyelination as the % area with demyelinated axons relative to endoneurial area [98, 99].

PK/PD models:

The basic principle of PK/PD modelling is to link PK and PD to establish and evaluate dose-concentration-response relationships, and subsequently describe and predict the time course of action of a drug dose [71]. The PK/PD model is particularly useful when evaluating capsaicin toxicity risk, because its PK profiles and PD concentrations are essential factors in determining safety and efficacy [72, 73]. Prodrug/capsaicin PK profiles and PD concentrations can be determined in parallel with sciatic nerve blockade and systemic toxicity assessments. At predetermined intervals, each rat can undergo sensory and motor neurobehavioral tests to evaluate for axon blockade.

Blood can be obtained from the tail immediately after completing neurobehavioral tests. At predetermined intervals, tail vein blood samples (0.1 mL) can be collected using a heparinized 1 mL disposable syringe after completing neurobehavioral tests in each rat. Multiple blood collections can be spaced far enough apart to prevent anemia, shock due to low blood volume, and distress in animals. Blood samples can be immediately mixed with an equal volume of modified Hank's balanced salt solution (pH 7.4) containing 500 units/mL heparin and centrifuge at 3000 g for 5 min. The resulting supernatant can be extracted with nine volumes of methanol containing 10% volume/volume acetic acid and ultrafilter through a Vivaspin 500 (MWCO 5000, VivaScience AG). The filtrate can be lyophilized and redissolved in 20 mM heptafuluorobutylic acid in 10 mM ammonium formate (pH 4.0) for HPLC to determine prodrug/capsaicin concentrations.

Plasma prodrugs/capsaicin concentration-time courses can be plotted to generate PK profiles. The lowest plasma prodrugs/capsaicin concentration that causes signs of systemic toxicity can be defined as the prodrugs/capsaicin toxic level in rats. The lowest plasma prodrugs/capsaicin concentration that produces (complete nociceptive-selective) sciatic nerve block can be defined as the prodrugs/capsaicin therapeutic level in rats. For each group, an equal number of male and female rats can be used for this test.

### Example 2: Galacturonic Acid-Capsaicin Prodrug for Prolonged Nociceptive-Selective Nerve Blockade

Injection of conventional amino-amide and amino-ester local anesthetics around nerves can effectively inhibit peripheral nerve signal transduction and relieve pain [1]. However, clinically available local anesthetics affects all nerve fiber types upon local application, impeding action potential propagation in nociceptive, sensory, and motor neurons and inducing unnecessary motor paralysis in the affected region [2-3]. As a consequence, patients have to deal with varying degrees of muscle weakness for adequate pain relief. This is particularly problematic for postoperative pain management that typically lasts for 5-7 days and long-term chronic pain management for as long as 12 weeks [4]. Therefore, there is a clinical need to develop a nerve blocking agent that can produce nociceptive-selective nerve blockade (i.e., a selective nerve blockade that blocks nociceptive signaling without motor nerve blockade and disruption of proprioception and touch sensation) lasting 7-14 days with a single perineural injection with minimal local or systemic side effects.

The transient receptor potential vanilloid-1 (TRPV1) receptor is a key nociceptive channel expressed in neurons of various sensory ganglia and is pivotal. It plays a vital role in transmitting sensory information from the periphery to the somatosensory cortex [5]. It is activated by various stimuli including high temperatures, low pH, and a range of natural products (resiniferatoxin, capsaicin, gingerol, etc.), as well as various venoms [6-7]. Recent studies have focused on the structure of TRPV1 and its potential for developing innovative therapeutics for diseases in which TRPV1 is involved, such as pain, cancer, and neurodegenerative diseases [8]. Capsaicin, the active component of chili peppers, is an agonist for the TRPV1 receptor and can bind to residue Tyr511 to open the TRPV1 receptors on primary afferent C-fibers. Capsaicin and other TRPV1 agonists can help relieve pain because prolonged or repeated activation of TRPV1 can lead to receptor desensitization and insensitivity to subsequent stimuli and lead to the depletion of certain neurotransmitters, such as substance P, which are involved in transmitting pain signals [9]. Since TRPV1 is expressed primarily on the central and peripheral terminals of nociceptive neurons in axons and the dorsal root ganglion cells (DRGs) [10], capsaicin selectively acts on nociceptive signaling without motor nerve blockade or disruption of proprioception and touch sensation. As a consequence, capsaicin is a potential nociceptive-selective blocking agent for nociceptive signals. The use of capsaicin to inhibit nociceptive signaling without impairing motor function has recently been reported [11]. Perineural injection of capsaicin with QX-314, a quaternary lidocaine derivative with obligate positive charges, produces a nociceptive-selective axon blockade lasting 2 hours in rats. However, 2 hours is relatively short, making this formulation clinically inadequate for post-operative pain and chronic pain syndromes lasting days to weeks.

Applying capsaicin for prolonged duration nociceptive-selective axon blockade poses two major challenges: i) Insufficient drug permeation due to peripheral nerve barriers (PNBs). In order to act on nociceptive sensory neurons, the perineurally injected capsaicin must first cross the tight junction forming restrictive perineurium composed of multiple concentric layers of specialized epithelioid myofibroblasts [12]. It is well established that a very small percentage (<1%) of local anesthetics injected subcutaneously penetrate the perineurium to subsequently modulate axonal signal transduction [13]. These drugs are significantly adsorbed into adjacent tissues and/or taken up into the systemic circulation. ii) Dose limitation due to the potential local and systemic drug toxicity. Local toxic effects include irritation, redness, and burning sensation at the injection site, while systemic adverse effects can lead to dizziness, nausea, vomiting, hypotension, tachycardia, seizures, and respiratory depression [14]. Moreover, capsaicin toxicity can cause myotoxicity and neurotoxicity, leading to muscle weakness, poor stamina, and lack of muscle control, particularly with prolonged use or high doses [15].

Efforts have been made to increase the injection dose of capsaicin while reducing its toxicity. These efforts include the development of capsaicin prodrugs, such as Vocapsaicin [16-17], and drug delivery systems [18-21] that can achieve sustained release of capsaicin to improve capsaicin's pharmacokinetic profile. However, there is currently no research reported on improving capsaicin permeation through the PNBs.

Peripheral nerves are metabolically active. Sugar molecules, such as glucose, galactose, mannose, can gain access into peripheral nerve endoneurium from the blood circulation or interstitial fluid across restrictive cellular barriers [22]. Perineurial cells and endothelial cells that form the perineurium and blood-nerve barrier (BNB) express multiple glucose transporters (GLUTs), with glucose transporter protein type 1 (GLUT1) and glucose transporter protein type 3 (GLUT3) being the most prevalent [23-24]. GLUT1 is predominantly found in endoneurial capillaries and the perineurium, while GLUT3 is primarily located in myelinated fibers, endoneurial capillaries, and the perineurium [25]. GLUTs facilitate the transport of sugar molecules across these PNBs by carrier-mediated mechanisms [26-30].

This study developed sugar-capsaicin prodrugs, in which capsaicin is covalently linked to a sugar molecule through hydrolysable bonds. The hypothesis is that the transport of sugar-capsaicin prodrugs across PNBs is mediated by facilitative GLUTs, with resultant increased prodrug permeability into peripheral nerves. Following PNB transport into the endoneurium, hydrolysis of a specific sugar-capsaicin prodrug bond occurs. This process converts the inactive prodrug to active capsaicin, thereby inducing a nociceptive-selective axon blockade. Of note, the prodrug bond hydrolysis is a slow process that results in sustained release of active capsaicin over time.

This approach ensures a consistent supply of drug levels necessary for maintaining a prolonged nociceptive-selective axon blockade. Conversely, it prevents the initial release of a local drug bolus, mitigating the potential for drug-related local and systemic adverse effects. With this hypothesis, sugar-capsaicin prodrugs can facilitate safer injection of higher capsaicin doses to produce prolonged duration nociceptive-selective axon blockade with limited side effects.

### Materials and Methods

### Materials:

All chemicals and solvents of the highest purity available were used as purchased. 1,2,3,4-di-o-isopropylidene-α-d-galacturonic acid was purchased from Combi-Blocks, Inc. Capsaicin, methoxypolyethylene glycol amine (Mn=750), and 1,2,3,4-di-o-isopropylidene-a-d-galactopyranose were supplied by Sigma-Aldrich (St. Louis, MO). DMAP, DCC, silica gels, PBS buffer (pH 7.4), methanol-d₄ (100%, 99.96 atom % D), FITC, Tissue-Tek O.C.T. Compound, Glc-UDP-FL, and other organic solvents were purchased from VWR International Ltd (Radnor, PA).

### Synthesis and Characterization of GalA-CAP Prodrug:

Dicyclohexylcarbodiimide (DCC, 376 mg, 1.82 mmol) was added to a stirring solution of capsaicin (2, 555 mg, 1.82 mmol), 4-Dimethylaminopyridine (DMAP, 278 mg, 2.27 mmol) and 1,2,3,4-di-o-isopropylidene-α-d-galacturonic acid (1, 500 mg, 1.82 mmol) in anhydrous dichloromethane (DCM). The reaction mixture was stirred at room temperature overnight. The solvent was removed under vacuum and the crude product was partitioned between ethyl acetate and water. The organic phase was dried on Na₂SO₄, filtered and then concentrated in vacuum. The crude product was purified using flash chromatography on silica gel eluting with ethyl acetate, obtaining 1,2,3,4-di-o-isopropylidene-α-d-galacturonic acid-capsaicin ester (3) as a yellow oil (920 mg, 90% yield).

¹H-NMR (500 MHz, methanol-d₄) δ (ppm) 0.89 (d, 6H, 1′-H); 0.99 (d, 6H, 1-H); 1.20 (m, 2H, h-H); 1.31/1.40 (d, 12H, r-H); 1.66 (m, 2H, g-H); 2.02 (m, 2H, i-H); 2.26 (m, 2H, f-H); 3.83 (s, 3H, d-H); 4.38 (s, 2H, e-H); 4.52 (q, H, o-H); 4.72 (d, H, n-H); 4.75-4.81 (m, 2H, p, q-H); 5.34-5.42 (m, 2H, j, k-H); 5.64 (d, H, m-H); 6.90 (d, H, c-H); 7.02-7.05 (m, 2H, a, b-H); ¹³C-NMR (125 MHz, methanol-d₄) δ (ppm) 21.64 (C17′, dihydrocapsaicin); 21.74 (C17); 23.58 (C24); 25.2 (C12); 25.71 (C11′, dihydrocapsaicin); 27 (C14′, dihydrocapsaicin); 27.74 (C16′, dihydrocapsaicin); 35.60 (C10); 35.73 (C10′, dihydrocapsaicin); 38.76 (C15′, dihydrocapsaicin); 42.41 (C8); 54.99 (C7); 68.52 (C21); 70.35 (C19); 70.82 (C20); 72.04 (C22); 96.62 (C18); 109.05 (C26); 109.89 (C25); 111.93 (C5); 119.51 (C2); 112.28 (C3); 126.53 (C4); 137.53 (C15); 138.34 (C1); 151.14 (C6); 166.82 (C23); 174.7 (C9); MS (m/z) calculated for C₃₀H₄₃NO₉ [M]=561.29; found 562.312 [M+H]⁺; 584.209 [M+Na]⁺; 600.267 [M+K]⁺.

To a stirred solution of 3 (920 mg, 1.60 mmol) in DCM (10 mL), trifluoroacetic acid (5 mL) was added, and the mixture was stirred at room temperature for 48 h. After the reaction was completed, 10 mL of water was added. The organic layer was dried over sodium sulfate, filtered, concentrated, and submitted to a flash chromatography on silica gel, eluting with DCM/MeOH (90/10). After the evaporation of solvent, the GalA-CAP prodrug was obtained as a brown solid (490 mg, 62% yield).

¹H-NMR (500 MHz, methanol-d₄) δ (ppm) 0.87 (d, 6H, 1′-H); 0.96 (d, 6H, 1-H); 1.17 (m, 2H, h-H); 1.64 (m, 2H, g-H); 2.0 (m, 2H, i-H); 2.24 (m, 2H, f-H); 3.8 (s, 3H, d-H); 4.38 (s, 2H, e-H); 3.34-5.30 (m, 5H, m, n, o, p, q-H); 5.37 (m, 2H, j, k-H); 6.88 (d, H, c-H); 7.02-7.05 (m, 2H, a, b-H); ¹³C-NMR (125 MHz, methanol-d₄) δ (ppm) 21.64 (C17′, dihydrocapsaicin); 21.74 (C17); 25.16 (C12); 25.71 (C11′, dihydrocapsaicin); 27 (C14′, dihydrocapsaicin); 27.74 (C16′, dihydrocapsaicin); 28.92 (C11); 29.31 (C13′, dihydrocapsaicin); 31.87 (C16); 35.60 (C10); 35.73 (C10′, dihydrocapsaicin); 38.76 (C15′, dihydrocapsaicin); 42.41 (C8); 54.99 (C7); 68.52 (C19); 70.37 (C21); 74.18 (C22); 93.18 (C18); 111.93 (C5); 119.51 (C2); 122.28 (C3); 126.53 (C4); 137.7 (C15); 138.34 (C1); 168.15 (C23); 174.78 (C9); MS (m/z) calculated for C₂₄H₃₅NO₉ [M]=481.23; found 504.298 [M+Na]⁺; 520.274 [M+K]⁺.

### Measurement of Chemical Stability:

10 mg of the prodrugs was dissolved in 10 mL of Milli-Q water accordingly and cultured at 37° C. on the hotplate. 100 μL of the samples were withdrawn at each time point (0 h, 1 h, 2 h, 4 h, 6 h, 10 h, 24 h, 48 h, 72 h, 96 h, 120 h, and 168 h) and submitted to UPLC with a photodiode array detector and HSS T3 (1.8 m, 2.1 mm×100 mm) column (Acquity UPLC System; Waters, Milford, MA, USA) for quantification immediately. 10 p L of the collected sample were injected into the HSS T3 column for analysis. For esterase stability studies, 20 or 120 μL of an esterase solution (0.5 units/mL) was added to 480 or 380 μL of PBS buffer to make the esterase solution at the concentration of 20 units/mL or 120 units/mL, respectively. The esterase solution was then added to 500 μL of a stock solution of the prodrugs (2 mg/mL) in PBS buffer preincubated at 37° C. 50 μL of the aliquots were removed after 10 minutes and immediately analyzed by UPLC as described above.

### Synthesis and Characterization of Gly-CAP Prodrug:

To a solution of the capsaicin (555 mg, 1.82 mmol), Boc-Glycine-OH (397 mg, 2.27 mmol), and DMAP (278 mg, 2.27 mmol) in DCM (6 mL) at room temperature were added DCC (376 mg, 1.82 mmol). The reaction mixture was stirred at room temperature for 12 h. The solvent was removed under vacuum and the crude product was partitioned between ethyl acetate and water. The organic layer was dried over anhydrous Na2SO4 and concentrated in vacuum; the following purification by silica gel chromatography (Hexene/ethyl acetate, 50/50) led to the crude BOC-Gly-capsaicin as a yellow solid. Subsequently, to a stirred solution of BOC-Gly-capsaicin in ethyl acetate (3 mL), 4 M hydrochloric acid in 1,4-dioxane solution (10 mL) was added, and the mixture was stirred at room temperature for 12 h. After the reaction was completed, the organic layer was concentrated in vacuo; the following purification by recrystallization in ethyl acetate, obtaining Gly-CAP prodrug as white solid (524 mg, 72% yield).

¹H-NMR (500 MHz, methanol-d₄) δ (ppm) 0.81 (d, 6H, 1′-H); 0.91 (d, 6H, 1-H); 1.16 (m, 2H, h-H); 1.64 (m, 2H, g-H); 1.94 (m, 2H, i-H); 2.25 (m, 2H, f-H); 3.82 (s, 3H, d-H); 4.15 (s, 2H, m-H); 4.36 (s, 2H, e-H); 5.36 (m, 2H, j, k-H); 6.91 (d, H, a-H); 7.07 (d, 2H, b, c-H); ¹³C-NMR (125 MHz, methanol-d₄) δ (ppm) 20.15 (C17′, dihydrocapsaicin); 20.24 (C17); 23.65 (C12); 24.17 (C11′, dihydrocapsaicin); 25.48 (C14′, dihydrocapsaicin); 26.22 (C16′, dihydrocapsaicin); 27.43 (C11); 27.80 (C13′, dihydrocapsaicin); 29.37 (C16); 30.36 (C10); 34.07 (C10′, dihydrocapsaicin); 37.24 (C15′, dihydrocapsaicin); 40.86 (C8); 53.58 (C7); 110.21 (C5); 117.82 (C2); 120.43 (C3); 124.97 (C14); 136.40 (C4); 137.42 (C15); 149.29 (C6); 164.07 (C18); 174.3 (C9); MS (m/z) calculated for C₂₀H₃₀N₂O₄ [M]=362.22; found 363.196 [M+H]⁺; 385.174 [M+Na]⁺; 401.140 [M+K]⁺.

### Synthesis and Characterization of Gal-CAP Prodrug:

A solution of p-toluenesulfonyl chloride (549 mg, 2.88 mmol) in anhydrous DCM (3 mL) was added to a cooled (0° C.) solution of 1,2,3,4-di-o-isopropylidene-α-d-galactopyranose (4, 200 mg, 0.77 mmol) and DMAP (179 mg, 1.47 mmol) in anhydrous pyridine (3 mL). The solution was warmed to room temperature and stirred for 24 h. The solvent was removed under reduced pressure and the residue dissolved in water and extracted with ethyl acetate. The organic layer was washed with 0.5 M HCl and saturated sodium bicarbonate, brine, then dried over sodium sulfate, filtered, and evaporated to give the product (5). The product (5) was then used directly in the next step without further purification.

Compound 5 (400 mg, 0.97 mmol) was added to a pressure glassware containing capsaicin (400 mg, 1.31 mmol), K₂CO₃ (500 mg, 3.62 mmol) and 10 mL DMSO, and stirred for 48 h at 95° C. The solvent was removed under heating and vacuum, and the crude product was washed with water, extracted with ethyl acetate and submitted to a flash chromatography on silica gel (ethyl acetate/hexane=50/50) to give the product (6, 20% yield).

¹H-NMR (500 MHz, methanol-d₄) δ (ppm) 0.87 (d, 6H, 1′-H); 0.95 (d, 6H, 1-H); 1.16 (m, 2H, h-H); 1.31/1.40 (d, 12H, r-H); 1.61 (m, 2H, g-H); 1.97 (m, 2H, i-H); 2.21 (m, 2H, f-H); 3.81 (s, 3H, d-H); 4.27 (s, 2H, e-H); 4.02/4.64 (m, 2H, s-H); 4.13-4.36 (m, 4H, p, q, n, o-H); 5.30-5.39 (m, 2H, j, k-H); 5.49 (d, H, m-H); 6.69-6.92 (m, 3H, a, b, c-H); ¹³C-NMR (125 MHz, methanol-d₄) δ (ppm) 21.66 (C17′, dihydrocapsaicin); 21.74 (C17); 23.77 (C24); 25.2 (C12); 25.71 (C11′, dihydrocapsaicin); 27 (C14′, dihydrocapsaicin); 27.74 (C16′, dihydrocapsaicin); 28.92 (C11); 29.31 (C13′, dihydrocapsaicin); 31.87 (C16); 35.60 (C10); 35.73 (C10′, dihydrocapsaicin); 38.76 (C15′, dihydrocapsaicin); 42.41 (C8); 55.32 (C7); 66.13 (C23); 68.27 (C21); 70.66 (C19); 70.97 (C20); 96.39 (C18); 108.50 (C5); 109.08 (C2); 114.68 (C26); 115.26 (C25); 120.02 (C3); 126.48 (C4); 132.77 (C14); 137.73 (C15); 147.29 (C1); 150.01 (C6); 174.57 (C9); MS (m/z) calculated for C₃₀H₄₅NO₈ [M]=547.31; found 570.271 [M+Na]⁺; 586.258 [M+K]⁺.

To a stirred solution of 6 (100 mg, 0.13 mmol) in DCM (10 mL), trifluoroacetic acid (5 mL) was added, and the mixture was stirred at room temperature for 48 h. After the reaction, 10 mL of water was added. The organic layer was dried over sodium sulfate, filtered, concentrated, and submitted to a flash chromatography on silica gel (ethyl acetate/hexane=50/50 to ethyl acetate). The product portions were collected and evaporated to give the product as a brown solid (82% yield).

¹H-NMR (500 MHz, methanol-d₄) δ (ppm) 0.84 (d, 6H, 1′-H); 0.92 (d, 6H, 1-H); 1.12 (m, 2H, h-H); 1.59 (m, 2H, g-H); 1.96 (m, 2H, i-H); 2.19 (m, 2H, f-H); 3.78 (s, 3H, d-H); 4.25 (s, 2H, e-H); 3.20-5.20 (m, 5H, m, n, o, p, q-H); 5.32 (m, 2H, j, k-H); 6.79 (d, H, c-H); 6.84-6.92 (m, 2H, a, b-H); ¹³C-NMR (125 MHz, methanol-d₄) δ (ppm) 21.66 (C17′, dihydrocapsaicin); 21.74 (C17); 25.19 (C12); 25.71 (C11′, dihydrocapsaicin); 27 (C14′, dihydrocapsaicin); 27.74 (C16′, dihydrocapsaicin); 28.92 (C11); 29.31 (C13′, dihydrocapsaicin); 31.87 (C16); 35.60 (C10); 35.73 (C10′, dihydrocapsaicin); 38.76 (C15′, dihydrocapsaicin); 42.41 (C8); 55.13 (C7); 69.87 (C19); 72.95 (C21); 73.6 (C22); 97.37 (C18); 115.55 (C5); 117.91 (C2); 119.85 (C3); 126.49 (C4); 137.73 (C15); 174.70 (C9); MS (m/z) calculated for C24H37NO8 [M]=467.25; found 490.176 [M+Na]⁺; 506.154 [M+K]⁺.

### Animal Studies:

Animal studies were performed in accordance with protocols approved by the Institutional Animal Care and Use Committee of the University of Alabama (Protocol ID: 19-11-2992). Adult male Sprague-Dawley rats weighing 250-350 g (Charles River Laboratories, Wilmington, MA, USA) were group-housed under a 12 h/12 h light/dark cycle. Prior to injection, rats were briefly anesthetized with isoflurane through a facemask. Drug formulations were prepared by combining a Tween 20 solution with either free capsaicin or the GalA-CAP prodrug (both dissolved in methanol), followed by a vacuum process to evaporate the solvent. Prior to injection, the formulations were reconstituted in 0.3 mL of PBS buffer to create a 2.5% w/v Tween 20 solution containing a predetermined quantity of capsaicin or prodrug, achieved through vortex mixing. Solutions with 3 mg of capsaicin included 12 mg (4% w/v) of Tween 20 to enhance dissolution and avoid precipitation of capsaicin. The injection was conducted in the left leg by introducing a 23 G×¾″ needle posteromedially into the greater trochanter, pointing in an anteromedial direction. Once in contact with bone, 0.3 mL of the test formulation was injected. Neurobehavioral testing was performed at predetermined intervals after injection. The right leg served as a control for systemic toxicity.

Sensory nerve blockade was assessed by a modified hotplate test. The time that rats left their hindpaws on a hot plate (Model 39D Hot Plate Analgesia Meter, IITC Inc., Woodland Hills, CA) at 56° C. was measured by a stopwatch. The time is referred to as thermal latency. The paw was removed from the hotplate after 12 seconds to avoid injury to the rat. All experiments were repeated 3 times at each time point, and the average was used for data analysis. Motor function was tested by measuring extensor postural thrust. Briefly, the rat was placed on top of a digital balance and allowed to bear its weight on one hindpaw at a time. The maximum weight that the rat can bear without its ankle touching the balance was measured.

The duration of sensory block is the time it takes for the thermal latency to return to 7 seconds (the baseline thermal latency is approximately 2 seconds; therefore, 7 seconds represents the midpoint between maximal latency, set as the 12-second cutoff, and the baseline). The duration of motor block was defined as the time to return 50% of weight bearing.

### Molecular Docking:

Selected protein crystal complex (PDB: 7LPE) was repaired, hydrogenated, decrystallized, and energy optimized using the Protein Preparation module of Meastro, and to define binding pockets. The prodrug molecules were then optimized for 3D conformation and energy minimization using the LigPre module. Finally, the small molecules were flexibly docked into the binding pocket using the XP docking module of Glide. Top poses were used for visualization analysis by Pymol 2.5.

### In Vivo Distribution of FITC, PEG750-FL, Gal-FL and Glc-UDP-FL:

Gal-FL was synthesized by mixing FITC (12 mg, 0.031 mmol) with galactosamine hydrochloride (6.7 mg, 0.031 mmol) in 2 mL solvent (water/ethanol: 25/75). After the pH was adjusted to 7 using sodium bicarbonate, the mixture was stirred at room temperature for 24 hours. The mixture was used for further analysis and the preparation of the injection solution directly.

PEG₇₅₀-FL was synthesized by mixing FITC (10 mg, 25.7 μmol) with methoxy polyethylene glycol amine (Mn=750, 190 mg, 253 μmol) at an elevated temperature (80° C.) for 24 hours. The mixture was used for further analysis and the preparation of the injection solution directly.

A previous established but modified method was used to study the distribution of fluorescence in the nerve [61]. Under isoflurane-oxygen anesthesia, 0.3 mL of test solution (either FITC, Gal-FL, Glc-UDP-FL, or PEG₇₅₀-FL in PBS buffer) was injected beside the sciatic nerve. The absorption intensities were determined by a plate reader (SpectraMax i3x, Molecular Devices) to ensure that the same dose of four compounds were injected. After 4 hours, the sciatic nerves were harvested and embedded into OCT compound and frozen sections were prepared. A coverslip was placed, and the slides were imaged by Leica TCS SP2 confocal microscopy.

To measure the depth that the injected compounds penetrated the nerve. The quantitative analysis of the confocal fluorescence images was conducted by dividing the nerve structure into 10 concentric areas equally separated by distance based on the nerve shape. All the fluorescence intensities throughout the nerve were calculated by ImageJ.

### Tissue Harvesting and Histology:

Rats were sacrificed 14 days after the injection unless there was still a sensory block, and the sciatic nerve was harvested together with surrounding tissues to characterize the possible chronic inflammation and nerve degeneration. Muscle samples were fixed in 10% neutral buffered formalin and processed for histology (hematoxylin-eosin stained slides) using standard techniques. Muscle samples were stained with hematoxylin and eosin, and then scored for inflammation (0-4 points) and myotoxicity (0-6 points) [62].

To evaluate the neurotoxicity, the sciatic nerve samples were processed and fixed in Karnovsky's KII Solution (2.5% glutaraldehyde, 2.0% paraformaldehyde, 0.025% calcium chloride in 0.1 M cacodylate buffer, pH 7.4). Samples were treated with osmium tetroxide for post-fixation for 2 hours and were subsequently dehydrated in graded ethanol solutions for 10 min each (30%, 60%, 90%, 100%, 100%, 100%). Then, the nerves were infiltrated with ethanol/Epon resin mixtures (1:1, 1:2, 0:1). After being sectioned by a diamond knife through ultramicrotome, nerve sections of 800 nm were stained with toluidine blue, followed by light microscopy. Nerve sections of 100 nm were observed by transmission electron microscopy (TEM, Hitachi H-7650), and the diameter of the nerves was quantified by Image J software.

### Statistical Analysis:

Data are presented as means±SDs (n=3 in distribution measurement study, n=4 or 8 in neurobehavioral studies). The statistical differences between groups were tested by one-way analysis of variance (ANOVA) for multiple comparisons. Statistical analyses were performed with GraphPad Prism 9 software. Statistical significance was defined as a p<0.05.

### Results

### Synthesis and Characterization of the Galacturonic Acid-Capsaicin Prodrug:

This study designed and synthesized a prodrug by conjugating capsaicin (2) with galacturonic acid (i.e., the oxidized form of galactose) with a hydrolysable carboxylic acid ester bond. Galactose was selected as the sugar moiety due to its high binding affinities to GLUT1/3 (Km=17 mM for GLUT1, 8.5 mM for GLUT3). Galactose has been used as the active ingredient in prodrugs to enhance drug uptake by the brain and cancer cells through binding to the highly expressed GLUT1 in the blood brain barrier [31-32] and tumors [33]. The hydroxyl group at the carbon 6′-position of galactose is the most promising functional group for conjugation to drug molecules because this conjugation generally does not affect the affinity of galactose to GLUT transporters [34]. In this design, the carboxylic acid group at the C-1′ position of galacturonic acid was used to react with the hydroxyl group of capsaicin to form the carboxylic acid ester linkage.

Specifically, the galacturonic acid-capsaicin prodrug (GalA-CAP) was synthesized through a two-step reaction. The first step is a condensation reaction between capsaicin (2) and 1,2,3,4-di-o-isopropylidene-α-d-galacturonic acid (DIGA, 1) to synthesize a DIGA-capsaicin ester. Dicyclohexylcarbodiimide (DCC) was used as a condensing agent and 4-dimethylaminopyridine (DMAP) as a catalyst in this reaction. The second step involves removal of the ketal protection of hydroxyl groups in the DIGA-capsaicin ester using a trifluoroacetic acid solution, resulting in the formation of the GalA-CAP prodrug.

The GalA-CAP prodrug was characterized by NMR and MALDI-TOF mass spectrometry. In the ¹H NMR spectrum of the GalA-CAP prodrug, all peaks can be assigned to each proton of the prodrug molecule. In particular, the peak at 4.36 ppm is assigned to the N′-methylene (-CH₂) proton of the capsaicin moiety. After conjugation with the galacturonic acid, it shifted from 4.26 ppm of free capsaicin to 4.36 ppm of the prodrug. The peaks at 3.5-4.6 ppm are assigned to the methylene and methine proton of the conjugated galacturonic acid. The MALDI-TOF/MS spectrum in the positive ion mode displayed a predominant peak at m/z 504.298, which is assigned to the GalA-CAP ([M+Na]⁺). The purity of prodrug determined from ultra-performance liquid chromatography (UPLC) is 96.23%, while the detected impurities are capsaicin and dihydrocapsaicin (3.76%). These results demonstrated the successful synthesis of the designed prodrug.

Hydrolysis kinetics of GalA-CAP was evaluated in deionized (DI) water at 37° C. 10 mg of prodrug was suspended in 10 mL DI water and the sample was kept in a constant temperature bath at 37° C. At predetermined time intervals, the component of sample was analyzed and quantified via UPLC. Results demonstrated that capsaicin/dihydrocapsaicin was the decomposed product, indicating the prodrug converted to active capsaicin through the hydrolysis of the carboxylic acid ester bond. The hydrolysis was found to follow pseudo-first-order kinetics. The pseudo-first-order half-life (t_{1/2}) was calculated from the slope of linear plots of the logarithm of remaining prodrug against time. The t_{1/2} was determined to be 111.8 hours. The presence of esterase significantly accelerated the prodrug hydrolysis. Specifically, when there were 60 units/mL and 10 units/mL of esterase in 1 mL of PBS buffer, 91.1% and 87.3% of the prodrug were converted to capsaicin within 10 minutes, respectively (TABLE 1).

TABLE 1
Degradation of GalA-CAP and Gly-CAP prodrug in various incubation
media determined by UPLC. The concentration of the prodrugs is
1 mg/mL. The solution was stirred at 200 rpm and kept at 37° C.

| | | | | | | % of Degradation | |
|---|---|---|---|---|---|---|---|
| | | Half-lives | | | | after 10 mins | |
| Incubation media | | GalA-CAP | | Gly-CAP | | GalA-CAP | Gly-CAP |
| Water | | 111.8 | hours | 49.5 | hours | / | / |
| Esterase | 10 units/mL | <10 | mins | <10 | mins | 91.1 | 97.7 |
| (from porcine | | | | | | | |
| liver | 60 units/mL | <10 | mins | <10 | mins | 87.3 | 95.4 |

### Preparation of Injectable Prodrug Formulations:

By adding the sugar moiety, the hydrophilicity of the GalA-CAP prodrug (Log P=1.53) increased compared to capsaicin (Log P=3.66). However, it is still poorly water soluble. In order to make the injectable prodrug formulation, Tween 20 was used as s surfactant. Specifically, the GalA-CAP prodrug and capsaicin were dissolved in methanol with Tween 20 at room temperature. The mixture was then subjected to a vacuum process overnight at room temperature to remove the methanol. Subsequently, the mixture was suspended in PBS buffer to achieve the desired concentrations of the drug and Tween 20.

Dynamic light scattering (DLS) analysis revealed that the prodrug formed aggregates in the prepared formulation. The size of aggregates was found to be linearly correlated with the prodrug concentration. Specifically, the aggregate size increased from 842±293 nm to 4167±350 nm when the concentrations increased from 1.67 mg/mL to 10 mg/mL (equivalent to 0.5 to 3 mg in 0.3 mL PBS buffer for injection with 2.5% (w/v) Tween 20, FIG. 7).

### Nociceptive Axon Blockade and Systemic Toxicity:

The efficacy and safety for nociceptive-selective nerve blockade of the synthesized GalA-CAP prodrug was evaluated using a rat sciatic nerve block model. Male Sprague-Dawley rats (250-350 g) were injected at the left sciatic nerve with 0.3 mL of 1×PBS containing 7.5-12 mg Tween 20 and 0.5-3 mg GalA-CAP prodrug or capsaicin. Neurobehavioral tests were performed to determine the duration of functional deficits, including nociceptive (measure paw withdrawal latency using a hotplate test) and motor axon blockade (measure the maximum weight a rat could bear using a weight-bearing test), in both hindpaws. The duration of deficits on the injected (left, ipsilateral) side reflected the duration of nerve block. Deficits on the uninjected (right, contralateral) side and other possible side effects (respiratory distress or seizure) reflected undesired systemic distribution of the injected formulation.

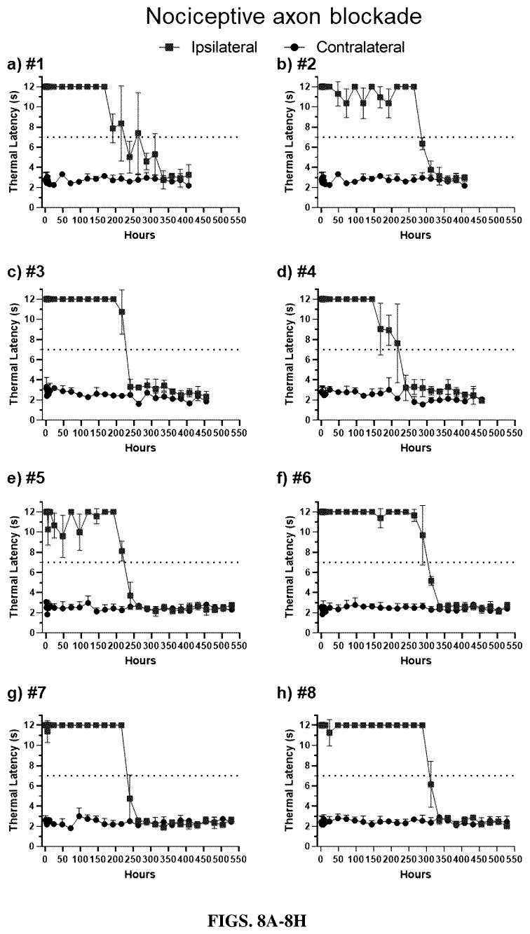

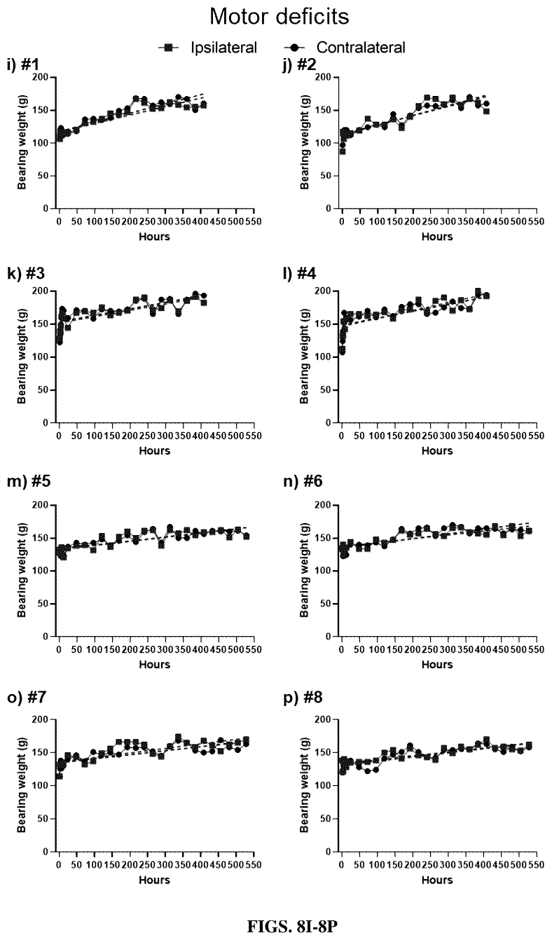

Groups of rats receiving sciatic nerve injections of both the GalA-CAP prodrug and free capsaicin showed dose-dependent increases in the frequency of successful nerve blocks and in the average duration of nerve block. Specifically, both groups achieved a 100% successful analgesic block (i.e. all rats tested showed thermal paw withdrawal latencies >7 seconds) at injection doses above 1 mg. In addition, the injection did not produce any motor deficits (i.e., from motor fascicular blockade) in the injected hind limb. A reversible nociceptive axon blockade was successfully induced following a single prodrug injection of 2 and 3 mg lasting for 150.0±76.8 and 234±37 hours, respectively. Importantly, the injection did not cause any capsaicin-related side effects, such as spontaneous pain behavior (e.g. flinching, licking), contralateral nociceptive axon blockade in the uninjected right hind limb, irreversible nociceptive axon blockade, seizure, or acute respiratory distress. Full thermal stimulus sensitivity of injected rats was restored after 13 days, as evidenced by a return to baseline thermal latency (FIGS. 8A-8H). No motor deficits were observed, as demonstrated by the consistent maximum bearing weight across both hind paws (FIGS. 8I-8P). However, increasing the dosage of GalA-CAP prodrug to 4 mg led to systemic toxicity and adverse effects, such as seizures observed in 2 out of 4 rats. The side effects are likely due to the higher systemic distribution of the metabolically converted capsaicin.

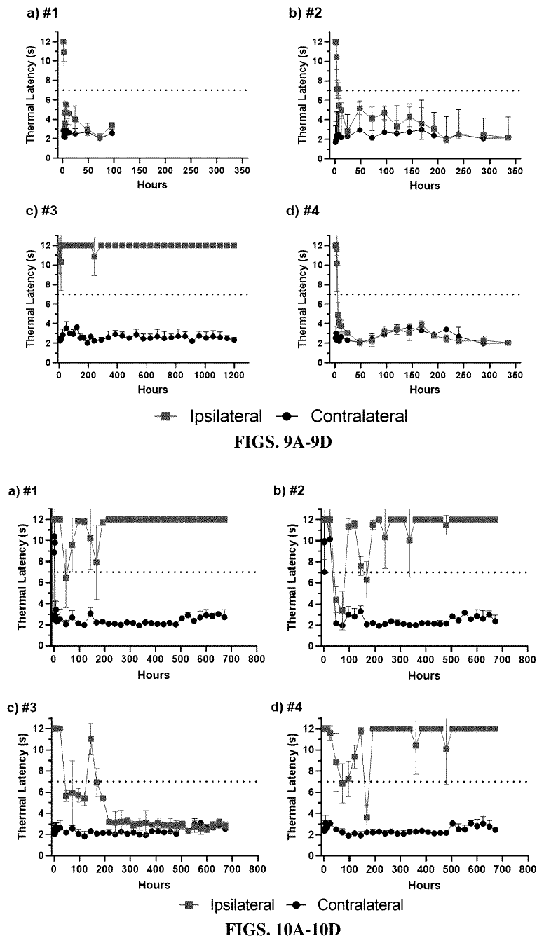

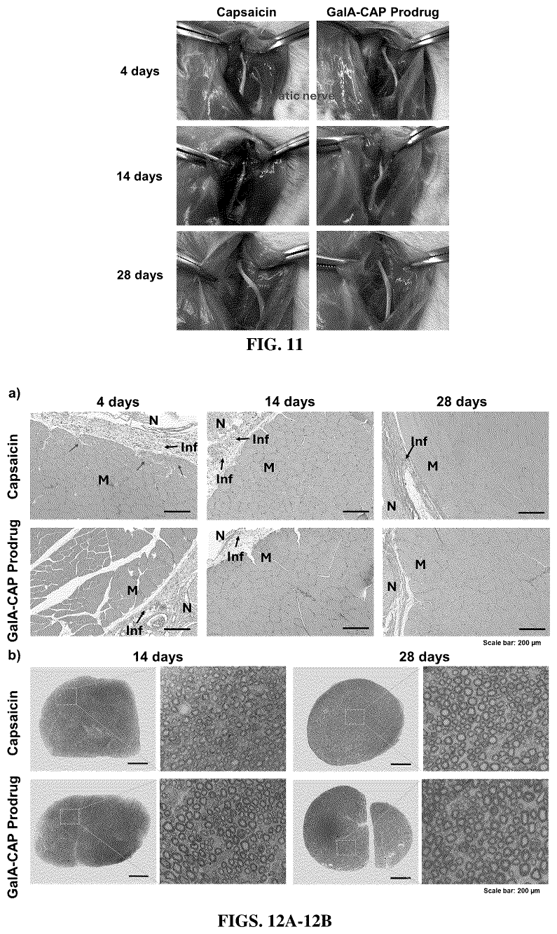

In contrast, injection of 2 and 3 mg of pure capsaicin produced a nociceptive axon block lasting for 4±2 and 30±12 hours, respectively. It was observed that all rats injected with 3 mg of pure capsaicin exhibited at least one systemic adverse effect (TABLE 2). Specifically, 75% developed acute respiratory distress, 25% experienced a seizure, 50% developed contralateral nociceptive axon block. Injection with 2 mg and 3 mg of capsaicin caused irreversible nociceptive axon blockade in 25% and 75% of animals in the injected hind limb, respectively, indicating capsaicin-induced nerve damage (FIGS. 9A-9D, FIGS. 10A-10D). The observation of irreversible nociceptive axon blockade aligns with previous reports of an initial transient thermal latency spike succeeded by a prolonged latency period that was never recovered [35].

TABLE 2
Side effects record of rats injected with capsaicin. The seizure
intensity was evaluated as follows : Stage 0, no response; Stage
1, ear and facial twitching; Stage 2, myoclonic jerks, convulsive
waves through the body; Stage 3, clonic convulsion with forelimb
clonus and rearing; Stage 4, clonic seizure with rolling over into
a side position side position, loss of postural control; Stage
5, generalized tonic-clonic seizures (TCS), tonic extension episode
and status epilepticus; and Stage 6, death within 30 min.

| | Dosage | |
|---|---|---|
| Drug | (mg) | Record of side effects |
| Capsaicin | 2 | #3: irreversible sensory nerve block |
| | 3 | #1: acute respiratory distress, contralateral |
| | | sensory nerve block, irreversible sensory nerve |
| | | block |
| | | #2: acute respiratory distress |
| | | #3: acute respiratory distress, contralateral |
| | | sensory nerve block, irreversible sensory nerve |
| | | block |
| | | #4: seizure (stage 5), irreversible sensory |
| | | nerve block |

### Tissue Reaction:

Conventional local anesthetics, particularly with prolonged duration, can be associated with myotoxicity and inflammation [36]. In order to study the local effects of prodrug and pure capsaicin injections, treated rats were euthanized 4 days (n=3), 14 days (n=4) and 28 days (n=4) after injection. The sciatic nerves and their surrounding tissues were harvested, sectioned, and stained for histologic evaluation. Hamstring muscles were processed and stained with hematoxylin-eosin (H&E). Epon-embedded semi-thin sciatic nerve sections were generated and stained with toluidine blue (gold-standard for peripheral nerve morphology).

It was observed that the surrounding tissues in rats injected with 2-3 mg of capsaicin appeared reddish and swollen (FIG. 11). Microscopic examination revealed a trace of perifascicular internalization of the nucleus (myotoxicity score=1) in one rat (out of 3) injected with 2 mg of capsaicin after 4 days (FIG. 12A). The cause of myotoxicity can be attributed to capsaicin, because previous studies using the same rat model have shown that injections of PBS buffer and Tween 20 with the tested concentration do not cause myotoxicity and neurotoxicity [37-39]. Mild inflammation was observed in tissues harvested from rats after 4 days of injection of 2 mg of capsaicin, with a median score of 3 (range [1-4]). In contrast, the surrounding tissues in rats injected with 2-3 mg of the GalA-CAP prodrug did not appear edematous or discolored, and there were no obvious signs of tissue injury. Microscopic examination did not reveal myotoxicity (median 0 for all groups, TABLE 3). Mild inflammation was observed in tissue harvested from rats after 4 days of injection of 2 mg of the GalA-CAP prodrug, with a median score of 1 (range [1-3]). There are no signs of inflammation in the deeper layers of the muscle. The trace of inflammation faded over time, and the score was lowered to 1 after 28 days of injecting 3 mg of the GalA-CAP prodrug or free capsaicin.

TABLE 3
Myotoxicity and inflammation scores. Inflammation score
range: 0-4; myotoxicity score range: 0-6. Data for inflammation
and myotoxicity are medians and range, and are compared
using the unpaired Manne-Whitney U test. This method
was selected because the data was ordinal.

| | Inflammation | | Myotoxicity | |
|---|---|---|---|---|
| Group | Score | | Score | |
| Capsaicin, 2 mg, 4 days | 3 | (1-4) | 0 | (0-1) |
| GalA-CAP Prodrug, 2 mg, 4 days | 1 | (1-3) | 0 | (0-0) |
| P value | 0.7000 | | >0.9999 | |
| Capsaicin, 2 mg, 14 days | 1.5 | (1-2) | 0 | (0-0) |
| GalA-CAP Prodrug, 2 mg, 14 days | 2 | (1-3) | 0 | (0-0) |
| P value | 0.6571 | | >0.9999 | |
| Capsaicin, 3 mg, 28 days | 1 | (0-1) | 0 | (0-0) |
| GalA-CAP Prodrug, 3 mg, 28 days | 1 | (0-2) | 0 | (0-0.5) |

| | | |
|---|---|---|
| P value | >0.9999 | >0.9999 |

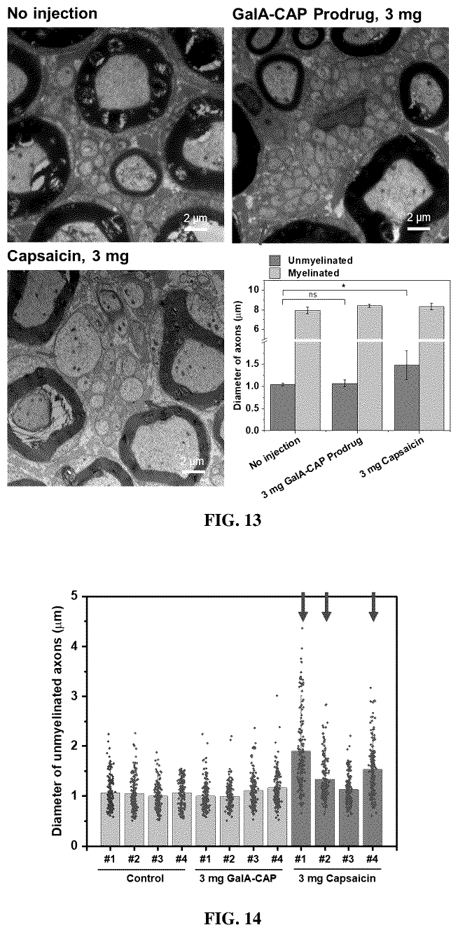

The study also observed myelinated axons in toluidine blue-stained sections (FIG. 12B). The myelinated nerves appeared normal, showing no signs of nerve degradation, with representative images provided. The sciatic nerves harvested from rats 28 days post-injection of 3 mg capsaicin and GalA-CAP prodrug were then examined by transmission electron microscopy (TEM) to better visualize the unmyelinated C-fibers, where TRPV1 is mostly expressed and targeted by the capsaicin. “No injection” controls were selected to better represent the original condition of the C-fibers and to avoid any potential confounding effects caused by the injection process. The hypertrophy of C-fibers was observed in nerves harvested from rats administered with 3 mg capsaicin (FIG. 13). The observed lesions and degeneration in the unmyelinated sensory axons align with the irreversible nociceptive axon blockade induced by the 3 mg capsaicin injection (FIGS. 10A-10D, FIG. 14). The damage to nociceptive axons caused by capsaicin is well-documented. Topical application of a high dosage of capsaicin could cause nearly complete degeneration of epidermal nerve fibers and the subepidermal neural plexus in treated skin [40]. Such deterioration of nerves could potentially be attributed to osmotic swelling of C-fibers stemming from unsustainable intracellular calcium concentrations [41]. However, the diameters of unmyelinated axons in rats injected with 3 mg of GalA-CAP prodrug were not significantly increased compared to those of the control group without injection, which could be attributed to the mild release of capsaicin by the prodrug. These findings, which align with the results from animal behavioral tests, highlight the impact of histological changes and underscore the importance of reversible nerve blockade in analgesic treatments.

### Enhanced Efficacy and Reduced Side Effects Due to Facilitated GLUTs Transport:

It was hypothesized that the enhanced efficacy and reduced side effects of the prodrug in nociceptive-selective nerve blockade are due to that the galacturonic acid moiety enhances prodrug permeability across the restrictive PNBs via carrier-mediated transport by the facilitative GLUTs. To confirm this hypothesis, the prodrug was co-injected at the rat sciatic nerve with the glucose and the GLUT inhibitor (KL-11743), respectively. Glucose has a comparable binding affinity for GLUTs (Km=17 mM for GLUT1, 11 mM for GLUT3) to galactose [34], and was previously reported to reduce the GLUT1-mediated uptake of the prodrugs by the brain when co-administrated with glucosyl ketoprofen and indomethacin prodrugs [42]. KL-11743 is a commercially available small-molecule inhibitor that impedes the function of class I glucose transporters (GLUT 1-4) both in vitro and in vivo [43]. Here they are used as competitive substrates for the prodrug because the preferential binding between glucose/KL-11743 and GLUTs can reduce the possibility of the prodrug binding to GLUT, and thus hindering GLUT transporter-mediated transport of prodrugs through the PNBs permeation.

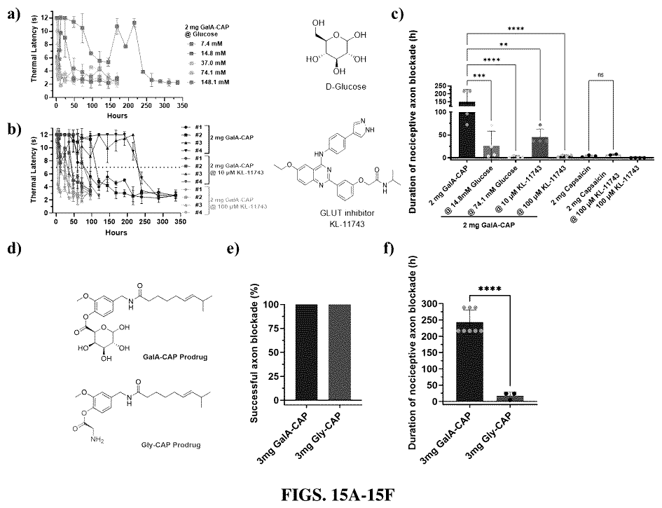

2 mg of the GalA-CAP prodrug was co-dissolved with varying doses of glucose or KL-11743 in 0.3 mL of PBS buffer (with 2.5% (w/v) Tween 20). The prepared formulation was administered at the sciatic nerve of rat. The duration of the thermal nociception block was reassessed. The duration of nerve block was significantly reduced with increasing glucose dose (FIG. 15A, FIG. 15C). Specifically, when 2 mg of the GalA-CAP prodrug was injected together with 14.8 mM (0.8 mg) glucose, the nociceptive axon blockade duration significantly decreased to 26.25±31.88 hours, compared to 150±76.84 hours in the absence of glucose, and this duration further reduced to 2.50±2.38 hours with the co-application of 74.1 mM (4 mg) glucose.

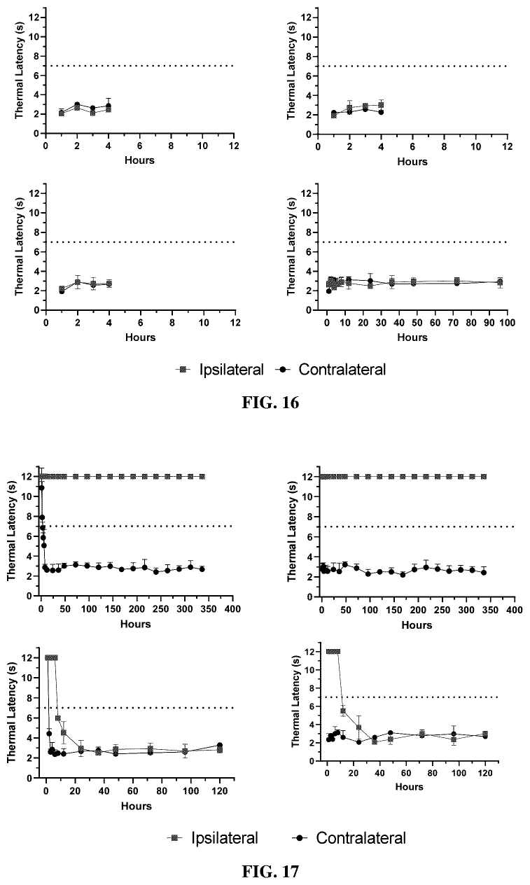

A similar trend of dose-dependent inhibition was observed when KL-11743 was co-administered with the prodrug (FIGS. 15B-15C). The nociceptive axon blockade durations were shortened to 45±18 hours and 4±2.31 hours when co-administered with 10 μM (1.57 μg) and 100 μM (15.7 μg) of KL-11743, respectively. These inhibition experimental results demonstrated that GLUT facilitates the transport of the GalA-CAP prodrug across the nerve barriers. Notably, the KL-11743 didn't cause any nerve blockade when injected solely and didn't affect the efficacy of capsaicin (FIG. 16, FIG. 17).

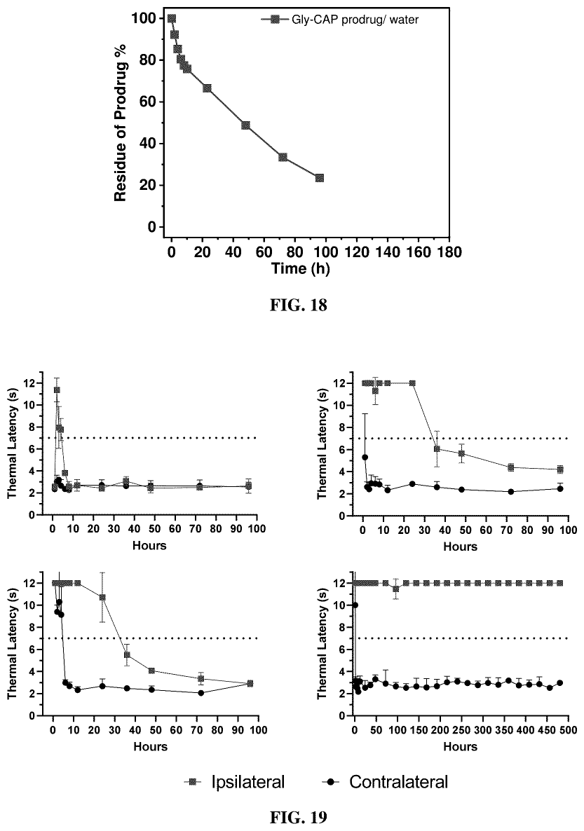

To further confirm the enhanced efficacy and reduced side effects of the GlaA-CAP prodrug due to facilitated GLUTs transport, the glycine-capsaicin prodrug (Gly-CAP) was synthesized. In Gly-CAP, glycine was linked to capsaicin through an ester bond (FIG. 15D, SCHEME 1). The Gly-CAP prodrug exhibited a similar hydrolysis profile in both water and enzyme environments as the GalA-CAP prodrug (FIG. 18, TABLE 1). However, Gly-CAP does not have a sugar moiety and may not be actively transported across the perineurium through the GLUT1 transporter. 3 mg of the Gly-CAP prodrug in 0.3 mL PBS (with 2.5% w/v Tween 20) was injected at the sciatic nerve of rats. The injection of 3 mg of the Gly-CAP prodrug demonstrated similar in vivo effects to pure capsaicin. It achieved a 100% successful nociceptive axon blockade (FIG. 15E). The average duration of nociceptive axon blockade in rats injected with 3 mg of Gly-CAP prodrug lasted for 17.33±11.55 hours, with one rat exhibiting irreversible nerve blockade (FIG. 15F, FIG. 19). Additionally, 1 in 4 rats experienced a seizure, and 2 rats developed contralateral nociceptive axon block.

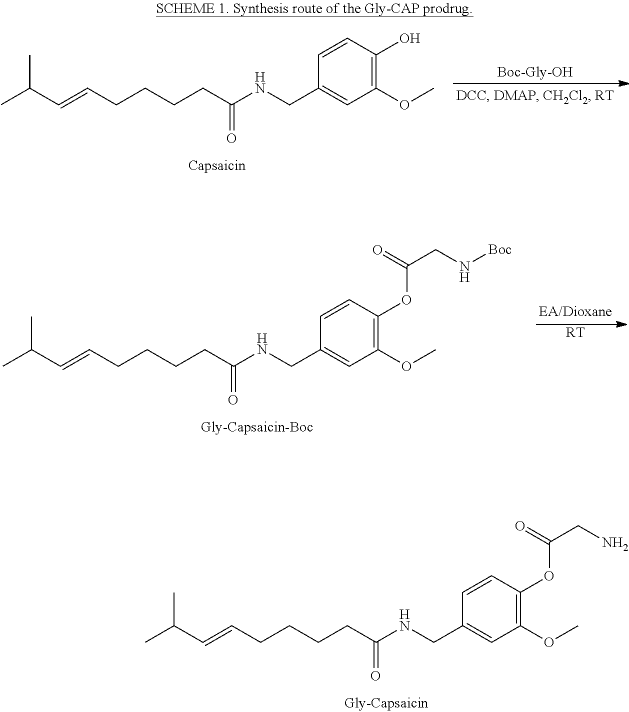

### Increased Uptake of Glycosyl Fluorescein by Nerve:

The enhanced prodrug permeation across the PNBs by GLUTs was also confirmed by the increased uptake of glycosyl fluorescein by nerves. The study compared the nerve permeation of galactosamine-fluorescein (Gal-FL, Mn=568, Log P=1.46), glucose-UDP-fluorescein (Glc-UDP-FL, Mn=1283, Log P=1.33), fluorescein isothiocyanate (FITC, Mn=389, Log P=3.77), and poly (ethylene glycol)₇₅₀-fluorescein (PEG₇₅₀-FL, Mn=1138, c Log P=−0.18). Gal-FL was synthesized by conjugating the primary amine at the C-2′ position of galactosamine to FITC. Gal-FL has the same galactose sugar moiety as GalA-CAP prodrug (Mn=481, Log P=1.53) and a similar molecular weight and log P value, so it could mimic the physicochemical properties and permeation behavior across the PNBs as the GalA-CAP prodrug. In order to confirm the role of GLUTs in facilitating the prodrug crossing PNBs, the study needed to demonstrate that prodrugs with different sugar moieties exhibit similar behavior. Therefore, Glc-UDP-FL, a commercially available compound, was selected due to its glucose sugar moiety. FITC was used as a negative control group. In addition to using FITC as a control group, PEG₇₅₀-FL was synthesized and employed as an additional control because it has a comparable molecular weight and hydrophilicity to Glc-UDP-FL, which helps minimize the impact of molecular weight and hydrophilicity on the permeation behavior of compounds across the PNBs.

0.3 mL of Gal-FL or Glc-UDP-FL or FITC or PEG₇₅₀-FL (15.6 μM) in PBS buffer was injected at the sciatic nerve. Four hours later, animals were euthanized, and the nerve and surrounding tissue were harvested. Frozen sections of the tissues were produced. Confocal microscopy imaging was used to track the location of fluorescein molecules.

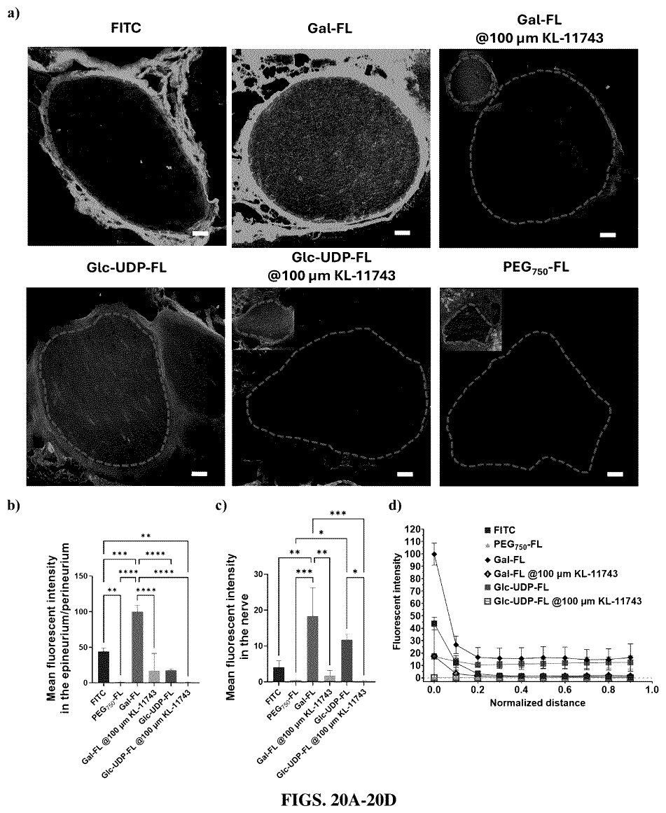

In rats administered with free FITC, fluorescence was predominantly external to the nerve, accruing in the epineurium and perineurium. In contrast, the fluorescence signal of Gal-FL and Glc-UDP-FL penetrated deep into the nerve (FIG. 20A). However, when Gal-FL and Glc-UDP-FL were co-administered with 100 μM KL-11743, a much lower fluorescence signal was observed inside the nerves. This indicates that KL-11743 inhibited the function of GLUTs and significantly impeded the permeation of Gal-FL and Glc-UDP-FL into the nerve. In addition, a much lower fluorescence signal was observed outside the nerves as well. This is because Gal-FL and Glc-UDP-FL are more hydrophilic than FITC, so they migrated more rapidly away from the injection site compared to FITC.

In rats administered with PEG₇₅₀-FL, weak fluorescent signals were observed within both the epineurium and endoneurium. This finding excluded the possibility that Gal-FL and Glc-UDP-FL facilitate the nerve permeation of FITC due to the increased molecular weight and hydrophilicity of FITC. Similar to the Glc-UDP-FL group, the limited accumulation of PEG₇₅₀-FL in the tissue around nerves could be attributed to its higher hydrophilicity, which causes it to quickly move away from the injection site after being administered.

Quantitative analysis showed that the mean fluorescent intensity in epineurium/perineurium and inside the nerve was significantly higher in animals injected with Gal-FL or Glc-UDP-FL than those injected with free FITC or PEG₇₅₀-FL. However, this increase disappeared when the Gal-FL and Glc-UDP-FL were administered along with a GLUT inhibitor (FIGS. 20B-20C). At a normalized distance of 0.1, Gal-FL group displayed the spike fluorescent intensity surrounding the nerve (range 0) and evenly distributed fluorescent signals within the nerve (ranges 0.1-0.9) (FIG. 20D), suggesting that the perineurium still remains the primary rate-limiting barrier for the diffusion of Gal-FL molecules.

Overall, these results indicate that sugar conjugation augments the trans-perineurial transport of fluorescein molecules through GLUT.

Enhanced Efficacy and Reduced Side Effects Due to Sustained Release of Capsaicin from the Less Active Prodrug:

It was hypothesized that the GalA-CAP prodrug is an inactive or less active form. Following prodrug transport across PNBs, the inactive or less active prodrug is gradually converted to active capsaicin through linker hydrolysis, leading to sustained drug release. This sustained drug release also contributes to enhanced efficacy and reduced side effects of the prodrug in nociceptive-selective nerve blockade. The in vitro prodrug hydrolysis assessment has confirmed the sustained drug release. Thus, here the study needed to confirm that the GalA-CAP prodrug is not as active as capsaicin in nerve blocks. It was hypothesized that the GalA-CAP prodrug is an inactive or less active form.

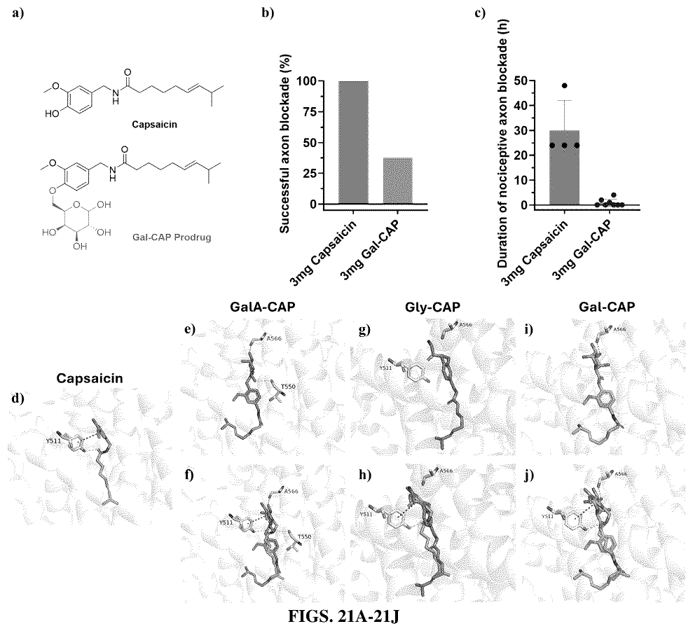

The study synthesized the galactose-capsaicin prodrug (Gal-CAP), in which the galactose was linked to capsaicin through an ether bond that is not hydrolysable (SCHEME 2). This modification preserves the physicochemical properties of the GalA-CAP prodrug while preventing esterase-mediated hydrolysis and subsequent conversion to capsaicin (FIG. 21A). The Gal-CAP prodrug was injected at the sciatic nerve of rats. Results showed that the Gal-CAP prodrug demonstrated less effectiveness compared to capsaicin, with only 37.5% (3 out of 8) of the rats injected with 3 mg of Gal-CAP prodrug showing successful nociceptive axon blockade (FIG. 21B). The average duration of nociceptive axon blockade in rats injected with 3 mg of prodrug only lasted for 0.88±1.46 hours (FIG. 21C, FIGS. 22A-22H).

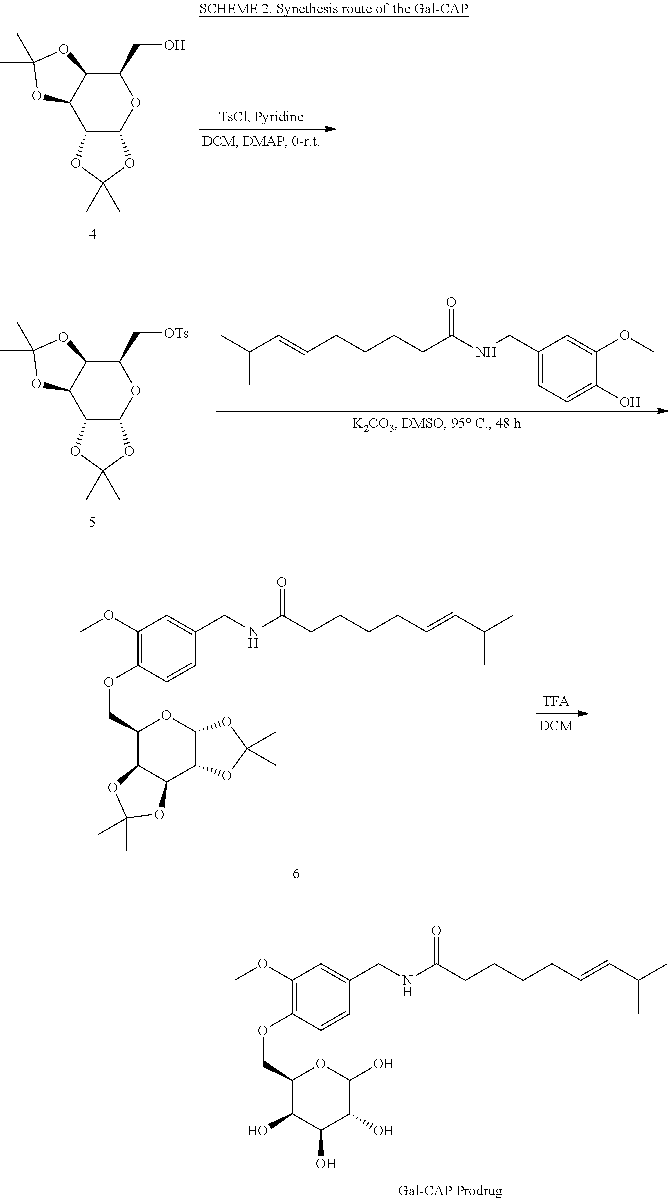

The results from molecular docking studies also confirmed these experimental findings. Instead of π-π stacking interactions with residue Tyr511 of the TRPV1 receptor by capsaicin (FIG. 21D), the GalA-CAP and Gal-CAP prodrugs, as well as the Gly-CAP prodrug, reveal unique and different binding modes with TRPV1 (FIGS. 21E-21J). These results suggest that the GalA-CAP and Gly-CAP prodrug require hydrolysis into active capsaicin via ester bond cleavage to exert their functional effects and should follow similar cascade mechanisms as capsaicin.

### Reduced Irritancy of GalA-CAP and Gal-CAP Prodrug:

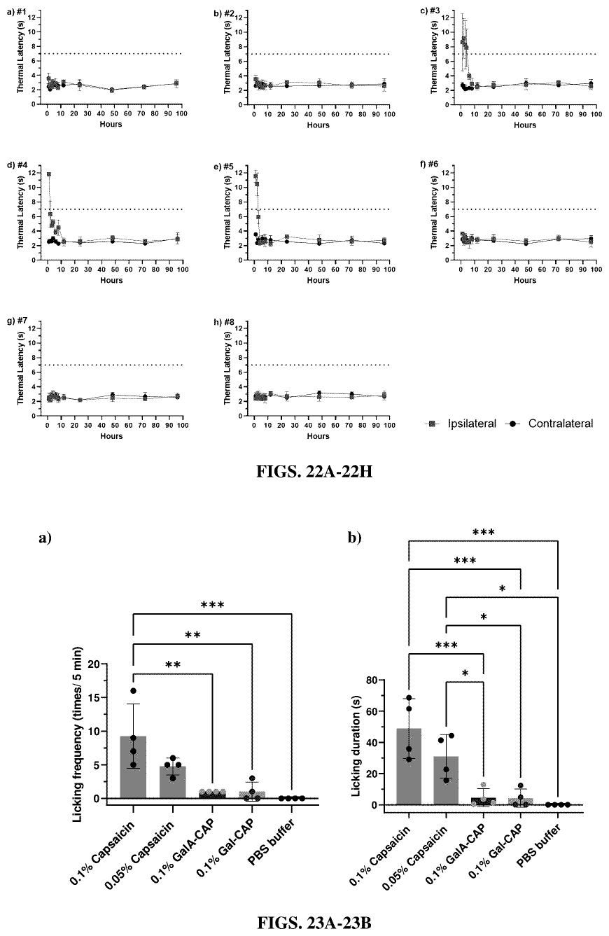

One limiting factor of capsaicin in its clinical use is the uncomfortable burning sensation post-application. As a result, pretreatment or co-administration with other local anesthetics is necessary to alleviate such uncomfortable burning sensations [44-45]. The study assessed the relative irritancy of prodrugs using a previously established animal behavioral test [45]. The frequency and duration of licking behavior were measured during the first 5 minutes after the intraplantar administration of 0.1% (w/v) capsaicin, 0.05% (w/v) capsaicin, 0.1% (w/v) GalA-CAP prodrug (containing 0.063% (w/v) active capsaicin), 0.1% (w/v) Gal-CAP prodrug (containing 0.065% (w/v) active capsaicin), and 1×PBS buffer. The frequency of licking within 5 minutes after the injection of 0.1% GalA-CAP prodrug, 0.1% Gal-CAP prodrug decreased to 1±0 times (1, 1, 1, and 1 time for 4 rats, respectively) and 1±1.41 times (0, 0, 1, and 3 times for 4 rats, respectively). In contrast, the frequency of licking within 5 minutes after injection of 0.1% capsaicin and 0.05% capsaicin was 9.25±4.79 times (5, 7, 9, and 16 times for 4 rats, respectively) and 4.75±1.26 times (3, 5, 5, and 6 for 4 rats, respectively) (FIGS. 23A-23B). Likewise, the total duration of licking behavior was significantly reduced to 4.28±5.81 seconds and 4.55±5.77 seconds after the injection of 0.1% GalA-CAP prodrug, 0.1% Gal-CAP prodrug, respectively, compared to 48.77±19.23 seconds after the injection of 0.1% capsaicin and 31.00±14.03 seconds after the injection of 0.05% capsaicin. These results suggest that the conjugation with galacturonic acid or galactose substantially reduced the irritant properties of capsaicin.

### Discussion

There are no clinically available nociceptive-selective axon blocking drugs. Conventional amino-amide and amino-ester local anesthetics produce motor axonal signaling inhibition of approximately the same duration as in sensory axons, making these drugs clinically inadequate for post-operative or chronic pain management in weight-bearing limbs. The galacturonic acid-capsaicin prodrug has the potential to become the first clinically applicable long-duration nociceptive-selective local analgesic drug to treat chronic pain through a single injection.

This study presents the first evidence of GLUT-mediated peripheral nerve drug uptake. It is widely accepted that sugar-based prodrugs can bind to GLUTs and therefore enhance drug uptake by cells or permeation through biological barriers. For example, GLUTs facilitate the transport of sugar-based prodrugs from the blood circulation across the blood-brain barrier (BBB) into the brain [29, 46-47]. Dalpiaz et al. observed that glucose-conjugated dopamine interacted with GLUT1 and inhibited the transport of 3-O-methylglucose by human retinal pigment epithelium (HRPE) cells [48]. Fernindez et al. found that the dopamine derivative, substituted at position C-6 of glucose, showed high binding affinity to GLUT1 in human erythrocytes [49]. Halmos et al. found that a glucose-chlorambucil derivative inhibited the uptake of [¹⁴C] D-glucose by the GLUT1 transporter in human erythrocytes [50]. Similarly, in cancer treatment, Lin et al. reported that glycan-based paclitaxel prodrugs enhanced delivery to cancer cells (NPC-TWO1) through GLUTs [51]. However, it remains unclear whether GLUTs actively transport peripherally injected sugar-based prodrugs across PNBs into peripheral nerve endoneurium. The study showed that PNB-expressing GLUTs mediate sugar-based prodrug transport into peripheral nerve perineurium. As a result, there is increased uptake of peripherally injected drugs to inhibit axonal signal conduction, with longer duration of effect and reduced local and systemic adverse effects.

Enhancing local anesthetic permeability through PNBs has important clinical and scientific implications. Previous reports indicate that less than 1% of the local anesthetic injected perineurally penetrate the perineurium and ultimately act on the peripheral nerve axons [13]. Co-administration of local anesthetics with chemical permeation enhancers (CPEs) has been the most effective way to increase local anesthetic peripheral nerve bioavailability and thereby enhance its analgesic effect. CPEs can interact with the intercellular lipids through physical processes including extraction, fluidization, increased disorder, and phase separation, thereby increasing the flux of local anesthetics into peripheral nerves [24]. However, CPEs are generally corrosive and excessive amounts can cause nerve damage [52]. In contrast, the galacturonic acid-capsaicin prodrug uses constitutionally expressed endogenous GLUTs to enhance local anesthetic peripheral nerve permeability. This strategy supports enhanced local anesthetic drug permeability and retention in peripheral nerves without causing CPE-related side effects.

The clinical application of this capsaicin-based prodrug extends beyond its primary use as a nerve-blocking agent for pain management. Due to capsaicin's various biological effects, including antioxidant [53], antimicrobial [54], anti-inflammatory [55], anticancer [56] and anti-obesity properties [57-58], the prodrug could potentially be customized for a wide range of therapeutic interventions.

This prodrug approach is not limited to GLUTs but can also be applied to various other transporters expressed in PNBs, such as monocarboxylate transporters [59-60]. The prodrug strategy is not only applicable to capsaicin, but can be effectively applied to other local anesthetics like bupivacaine and lidocaine, as well as many anticancer drugs, antibiotic drugs, etc.

A nociceptive-selective axon blocking agent was developed that provided days of nociceptive-selective axon blockade with minimal local and systemic toxicity. This innovative design incorporates three crucial components: a capsaicin that selectively inhibits nociceptive sensory nerve signaling without compromising other sensory perceptions or motor function; a sugar moiety that enhances prodrug flux into nerves; and a degradable linker that ensures a controlled and sustained release of active capsaicin at a safe rate.

### Example 3: Synthesis and Characterization of a Glucuronic Acid-Linked Bupivacaine

### Synthesis and Characterization of a Glucuronic Acid-Linked Bupivacaine:

A study was conducted which synthesized a glucuronic acid-linked bupivacaine (GluA-Bup) by coupling bupivacaine to glucuronic acid via a secondary amide bond (SCHEME 3). The chemical structure of the synthesized glucuronic acid derivative of bupivacaine was confirmed using proton nuclear magnetic resonance spectroscopy (¹H NMR) and matrix-assisted laser desorption/ionization-time of flight (MALDI-TOF) mass spectrometry (MS). The purity of this derivative, as determined by ultra-performance liquid chromatography (UPLC), was found to be 96.24%.

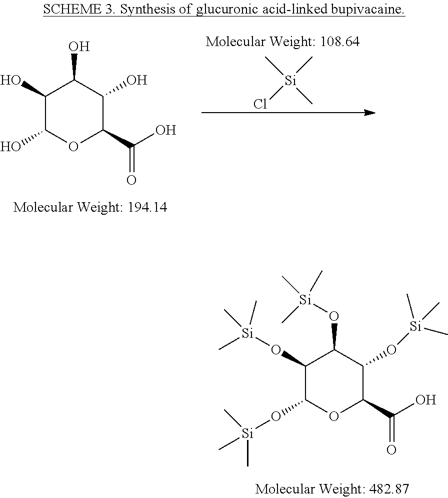

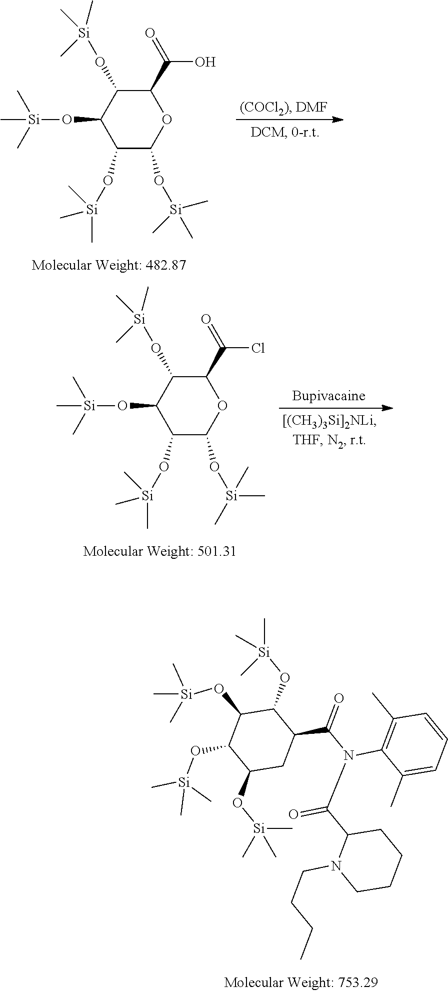

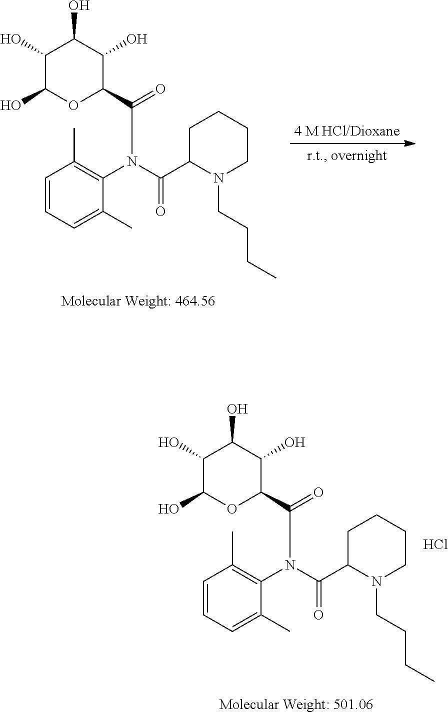 [SMILES: CCCCN1CCCCC1C(=O)N(C(=O)[C@H]1O[C@@H](O)[C@H](O)[C@@H](O)[C@@H]1O)c1c(C)cccc1C ; CCCCN1CCCCC1C(=O)N(C(=O)[C@H]1O[C@@H](O)[C@H](O)[C@@H](O)[C@@H]1O)c1c(C)cccc1C ; Cl]

Sensory-Preferred Nerve Blockade with Glucuronic Acid-Linked Bupivacaine:

In the investigation of glucuronic acid-linked bupivacaine (GluA-Bup), the study evaluated its in vivo efficacy and safety compared to traditional bupivacaine in blocking rat sciatic nerves. Adult male Sprague-Dawley rats weighing 250-350 g were injected peripherally at the left sciatic nerve with increasing doses of GluA-Bup or pure bupivacaine (dissolved in 0.3 mL of 1×PBS buffer). Neurobehavioral tests assessed the duration of functional deficits, including nociceptive (measuring paw withdrawal latency using a hotplate test) and motor axon blockade (measuring the maximum weight a rat could bear using a weight-bearing test).At a concentration of 8.65 mM, while pure bupivacaine failed to achieve a successful block, GluA-Bup achieved a 100% successful sensory blockade with a mean duration of 3.75+0.50 hours, without any motor deficits (FIGS. 24A-24C). At a higher concentration of 17.3 mM, pure bupivacaine achieved a 100% sensory block but with shorter mean durations of 1.75±0.29 hours and associated motor deficits averaging 1.375±0.48 hours (FIGS. 24A-24C). Conversely, GluA-Bup at the same concentration maintained a 100% sensory block with significantly prolonged mean durations of 13.0±2.58 hours and minimal motor involvement (1.625±0.25 hours, FIGS. 24A-24C). The GluA-Bup provided the sensory nerve blockade duration much greater than the motor nerve blockade, which demonstrated that the GluA-Bup can induce a biased sensory-preferred nerve blockade.

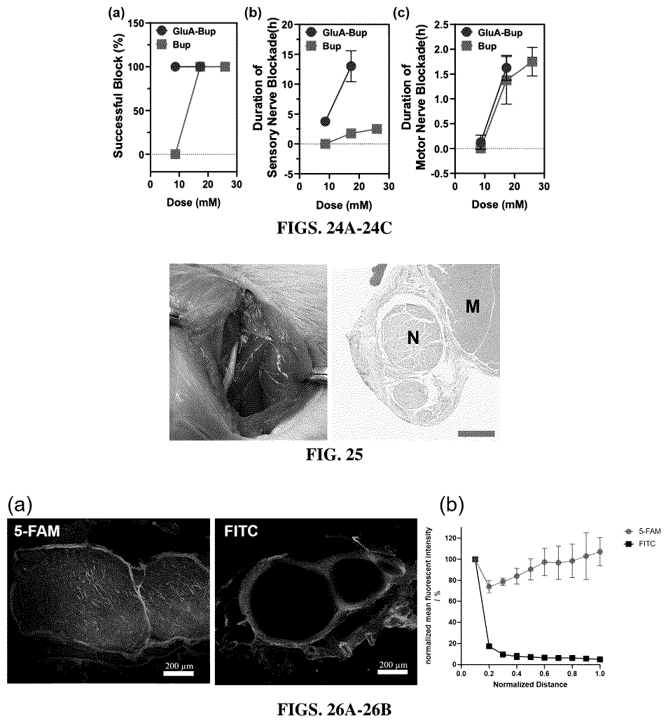

In order to study the local effects, treated rats were euthanized 4 days after injection. The sciatic nerves and their surrounding tissues were harvested, sectioned, and stained for histologic evaluation. Hamstring muscles were processed and stained with hematoxylin-eosin (H&E). In the rats administered 0.3 mL of 17.3 mM solution, the surrounding tissues showed no signs of edema or discoloration, indicating a lack of overt tissue damage (FIG. 25). Microscopic examination of these tissues did not reveal any significant myotoxicity or inflammatory responses. These findings suggest that GluA-Bup is well-tolerated at the administered doses, further supporting its potential for clinical use in peripheral nerve block applications without adverse local effects.

### Example 4: Carboxylic Acid Derivatives of Local Anesthetics with Enhanced Potency in Peripheral Nerve Block and Reduced Toxicity

Injection of clinically used amino-amide and amino-ester local anesthetics around peripheral nerves can effectively inhibit axonal signal transduction and relieve pain. However, less than 1% of the local anesthetics injected perineurally are able to cross the restrictive peripheral nerve barriers (PNBs), specifically the perineurium (an endothelial-like structure that encloses bundles of nerve fibers), and ultimately act on peripheral nerve axons. This limitation necessitates the administration of higher doses of local anesthetics, but even then, the analgesic effect remains insufficient. Furthermore, most injected local anesthetics are absorbed by adjacent tissues or taken up into the systemic circulation, leading to an increased risk of intrinsic muscle and nerve toxicity, as well as cardiovascular and neurological systemic side effects.

The goal of this study is to develop local anesthetics that are more bioavailable to peripheral nerve axons compared to the local anesthetics currently used in clinical practice. This can enhance the analgesic effect while minimizing side effects. Specifically, the aim is for more than 2% of the developed local anesthetics injected perineurally to penetrate the perineurium and act on peripheral nerve axons. By achieving this, the study can achieve equivalent or superior analgesic effects with lower doses. To pursue this goal, the study aims to develop carboxylic acid derivatives of local anesthetics, which are local anesthetics containing a carboxylic acid functional group. It is hypothesized that the carboxylic acid moiety can enhance the permeability of local anesthetics across the tight junction forming restrictive PNBs by using the carrier-mediated transport facilitated by monocarboxylate transporter 1 (MCT1). As a result, the carboxylic acid derivatives can enhance local anesthetic's bioavailability to peripheral nerve axons, reducing the amount of local anesthetic absorbed by neighboring tissues or entering the systemic circulation. This process can enhance the analgesic drug effect while minimizing adverse effects.

Preliminary observations provide support for the feasibility of the study. 5-Carboxyfluorescein (5-FAM), which is a carboxylic acid derivative of fluorescein isothiocyanate (FITC), was peripherally injected in a rat sciatic nerve model. These findings indicate that 5-FAM exhibits greater permeability into the nerve endoneurium across PNBs compared to FITC. This suggests that the carboxylic acid moiety facilitates the transport of FITC across tight junction forming restrictive PNBs, through carrier-mediated transport by the facilitative MCT1. In addition, a carboxylic acid derivative of capsaicin was synthesized, and it was demonstrated that a nociceptive-selective axon blockade can be achieved for up to 10 days following a single local injection of the carboxylic acid derivative of capsaicin at the rat sciatic nerve. This duration is 60 times longer than the effect achieved by an equivalent dose of capsaicin injection. Importantly, the study did not observe any local or systemic adverse effects associated with the carboxylic acid derivative of capsaicin, in contrast to equivalent capsaicin injections. This study aims to:
1) Develop carboxylic acid derivatives of local anesthetics with high binding affinity to MCT1 transporter, high in vitro blood-nerve barrier (BNB) and perineurium permeability, and high in vivo peripheral nerve barrier (PNB) permeability. This can be carried out by: a) designing and synthesizing carboxylic acid derivatives of different clinically used local anesthetics and structurally authenticating and characterizing the stability and cytotoxicity of the synthesized local anesthetics, b) screening carboxylic acid derivatives of local anesthetics by characterizing their binding affinity to the MCT1 transporter and selecting candidates that show binding affinity to the MCT1 transporter, c) screening the selected carboxylic acid derivatives of local anesthetics by evaluating their in vitro BNB and perineurium solute permeability and selecting candidates that have higher permeability than their corresponding unmodified local anesthetics, and d) screening the selected carboxylic acid derivatives of local anesthetics by evaluating their in vivo peripheral nerve permeability and selecting candidates that demonstrate an efficiency of >2% peripheral nerve permeability in an in vivo rat sciatic nerve model.
2) Perform in vivo evaluation of the efficacy and safety of carboxylic acid derivatives of local anesthetics for sciatic nerve blockade and assess the efficacy and duration of sciatic nerve block and the toxicity of carboxylic acid derivatives of local anesthetics in normal rats, nociceptive rat models of inflammatory pain, and traumatic nerve injury-induced neuropathic pain, compared with their corresponding unmodified local anesthetics. This can be carried out by: a) assessing the effects of carboxylic acid derivatives of local anesthetics injected peripherally in normal adult rats to evaluate dose-dependent: i) time course of sensory and motor nerve blockade, ii) local anesthetic plasma pharmacokinetics (PK) profiles and pharmacodynamics (PD) of nerve blockade, and iii) systemic local anesthetic-related toxicity, b) assessing the effects of carboxylic acid derivatives of local anesthetics injected peripherally on reflexive nociceptive withdrawal thresholds after unilateral hind paw inflammation or spared sciatic nerve injury, and c) assessing local tissue and organ reaction to carboxylic acid derivatives of local anesthetic administration (e.g., histological assessment) compared to their corresponding unmodified local anesthetics.

### Background

### Low Bioavailability of Local Anesthetics to Peripheral Nerve Axons:

Injection of local anesthetics around peripheral nerves to reversibly block neuronal voltage-gated sodium channels (VGSC) and to reversibly interrupt nerve impulse propagation and thereby relieve pain is used worldwide to treat acute and chronic pain. Clinically used amino-ester and amino-amide local anesthetics are effective, but they encounter 3 principal challenges: inadequate duration of action, local tissue reaction, and potential systemic toxicity [1-3]. They typically last for a few hours reflecting clearance of the molecule from the injection site. The duration of the block can be extended by perineural catheters, addition of adjuvant drugs to local anesthetics, or by formulating the product into sustained release systems, such as liposomes or nanoparticles [4-10]. However, clinically used amino-ester and amino-amide local anesthetics are intrinsically myotoxic [11, 12], leading to muscle weakness, poor stamina, and lack of muscle control, particularly with prolonged use or high doses [13]. Furthermore, once these local anesthetics are cleared from the injection site, they can have systemic effects, potentially causing cardiac dysfunction and neurological syndromes such as seizures [7, 14].

The key reason for the limitations mentioned above is the low bioavailability of local anesthetics to peripheral nerve axons. Peripheral nerve axons are surrounded by peripheral nerve barriers (PNBs) including epineurium, perineurium, and endoneurium. Particularly, the peripheral nerve perineurium, an endothelial-like structure that encloses bundles of nerve fibers, is the major rate-limiting step as local anesthetics permeate from the perineural injection site across the nerve structures and finally, diffuse toward their site of action [15, 16]. It is well established that only a very small percentage (<1%) of perineurally injected local anesthetics can penetrate the perineurium and subsequently modulate axonal signal transduction [15]. Most of the local anesthetics would be absorbed into adjacent tissues or enter the systemic circulation. These off-targeted local anesthetics can cause local and systemic drug toxicity.

### Monocarboxylate Solute Transport Through the PNBs is Mediated by Facilitative Monocarboxylate Transporter 1 (MCT1):

Endothelial cells that form the blood-brain barrier (BBB) express monocarboxylate transporters (MCTs), with monocarboxylate transporter 1 (MCT1) being the most prevalent. MCT1 facilitates the transport of a broad range of monocarboxylate solutes crossing the BBB, such as lactate, pyruvate, and acetoacetic acid through carrier-mediated mechanisms [17-19]. In addition to the transport of endogenous substrates, earlier studies have shown that carboxylate drugs such as valproic acid, benzoic acid, nicotinic acid, or beta-lactam antibiotics including benzylpenicillin, propicillin, and cefazolin can be transported into the brain via MCT1 [17, 20, 21]. Similarly, perineurial cells that form the perineurium express MCT1, and monocarboxylate solute transport through the PNBs is mediated by facilitative MCT1 [22-25].

### Carboxylic Acid Derivatives of Local Anesthetics:

The research goal is to develop local anesthetics that have better bioavailability compared to clinically used local anesthetics (e.g., more than 2% of perineurally injected local anesthetics can penetrate the perineurium to act on peripheral nerve axons). This would allow for equivalent or superior pain relief effects at lower doses. The result is a more effective and safer pain management solution for patients. In pursuit of this goal, the research approach involves the development of carboxylic acid derivatives of local anesthetics, in which a carboxylic acid group is incorporated into the local anesthetic. The hypothesis is that the transport of these derivatives across the perineurium is mediated by facilitative MCT1, and that this transportation mechanism can significantly increase the drug availability to peripheral nerve axons. This approach enables a higher proportion of the administered local anesthetic to produce the desired nerve block, while reducing the proportion that can cause local and systemic side effects.

### Significance

The proposed research is an important step towards the development of more effective clinically applicable drugs for acute post-operative and chronic pain management. If successful, this study could yield more potent nerve-blocking agents. This represents a significant advancement over currently used local anesthetics, which typically offer only short-duration pain relief and carry the potential risk of causing side effects. Adequate pain management with safe and effective carboxylic acid derivatives of local anesthetics can improve patient well-being and quality of life, and reduce the need for opioids. This in turn would decrease the significant risks, tolerance and addiction associated with opioids. Carboxylic acid derivatives of local anesthetics are also considered to have particular merit in veterinary patients. Applications in equine patients for surgery and the management of pain in hoof disorders such as laminitis represent great potential applications. Particularly, the use for regional infiltrations has evident applicability.

The carboxylic acid modification approach is not limited to local anesthetics and is applicable to other therapeutics for PNB disorders. This approach would support equivalent or superior therapeutic efficacy at lower injection doses. Furthermore, lower therapeutic doses would reduce the occurrence of local tissue and systemic adverse effects.

In addition to its practical implications for clinical practice, this study can also generate important fundamental knowledge on the structure-property relationships among emerging local anesthetics. For instance, the study can investigate how variations in chemical structure influence factors such as MCT-binding capacity, PNB permeability characteristics, and the capacity to produce sustained therapeutic pain relief.

Carrier-mediated transport of therapeutic agents across biological barriers has been widely used in the development and discovery of therapeutic agents. Perineurial cells that form the perineurium, a rate-limiting barrier for local anesthetics to cross before acting on peripheral nerve axons, express multiple MCTs, with MCT1 being the most prevalent. Previous studies have demonstrated that MCT1 facilitates the transportation of both endogenous substrates and carboxylate drugs across the BBB into the brain. Therefore, MCT1 is likely to promote carboxylic group-containing drugs to cross the perineurium and act on peripheral nerve axons.

This study presents the first evidence of MCT1-mediated drug uptake in peripheral nerves. Although the transport of carboxylic group-containing drugs across the BBB via MCT1 has been extensively studied [17, 20, 21], it remains unclear whether MCT1 actively transports these drugs injected peripherally across PNBs into the peripheral nerve endoneurium. This study demonstrates that MCT1, expressed in PNBs, can facilitate the transport of carboxylic group-containing anesthetics injected perineurally into the peripheral nerve endoneurium. As a result, there is an increased uptake of peripherally injected local anesthetics to inhibit signal conduction of neurons, leading to a longer duration of action and reduced local and systemic adverse effects.

Previous studies have shown that less than 1% of the local anesthetic injected perineurally is able to penetrate the perineurium and reach the peripheral nerve axons [15]. Co-administration of local anesthetics with chemical permeation enhancers (CPEs) has been the most effective way to increase local anesthetic peripheral nerve bioavailability and thereby enhance its analgesic effect. CPEs can interact with the intercellular lipids through physical processes including extraction, fluidization, increased disorder, and phase separation, thereby increasing the flux of local anesthetics into peripheral nerves [26]. However, CPEs are generally corrosive and excessive amounts can cause nerve damage [27]. In contrast, carboxylic acid derivatives of local anesthetics use constitutionally expressed endogenous MCT1 to enhance local anesthetic peripheral nerve permeability. This strategy supports enhanced local anesthetic drug permeability and retention in peripheral nerves without causing CPE-related side effects.

### Preliminary Studies

### MCT1 Transporter-Mediated Carboxylic Group-Containing Fluorescein Endoneurial Permeability In Vivo:

The study utilized a rat sciatic nerve in vivo permeability model to study MCT1 carrier-mediated drug permeability in peripheral nerves. Adult male Sprague-Dawley rats weighing 250-350 g were perineurally injected with 0.3 mL of Phosphate-Buffered Saline (PBS, 1x pH 7.4) solution containing FITC or carboxylic acid derivative of FITC (i.e., 5-Carboxyfluorescein (5 FAM)) at equal fluorescent intensities above the sciatic nerves. Four hours after injection, the rats were euthanized, and the sciatic nerves and surrounding tissues were harvested, frozen, sectioned, and visualized using a confocal microscope. As shown in FIG. 26A, in rats injected with FITC, fluorescence was observed in the surrounding epineurium. This observation indicates that FITC was unable to traverse the perineurium into the sciatic nerve endoneurium. It was observed that FITC accumulated in the hamstring muscles and associated adipose tissue around the sciatic nerve. In contrast, a uniform fluorescence distribution was observed within and outside the sciatic nerve endoneurium in rats injected with 5 FAM. There was no accumulation of fluorescein in the surrounding epineurium. Quantitative analysis showed that the fluorescence signal persisted deeper into the nerves of rats injected with 5 FAM (FIG. 26B). The results showed that MCT1 can actively transport peripherally injected carboxylic group-containing fluorescein across PNBs into peripheral nerve endoneurium.

### Synthesis and Characterization of a Carboxylic Acid Derivative of Capsaicin:

The study synthesized a carboxylic acid derivative of capsaicin by linking capsaicin to succinic acid through an ester bond (SCHEME 4) [28]. The study confirmed the chemical structure of the synthesized carboxylic acid derivative of capsaicin by proton nuclear magnetic resonance spectroscopy (¹H NMR) and matrix-assisted laser desorption/ionization-time of flight (MALDI-TOF) mass spectrometry (MS). The purity of the derivative determined from ultra-performance liquid chromatography (UPLC) is above 95%.

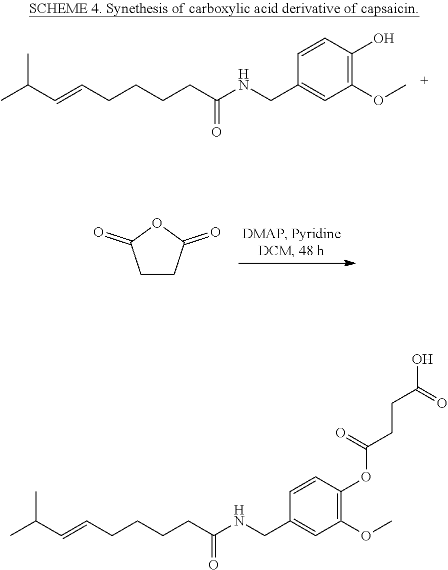

Thermal Nociception Blockade but not Motor Blockade with the Carboxylic Acid Derivative of Capsaicin:

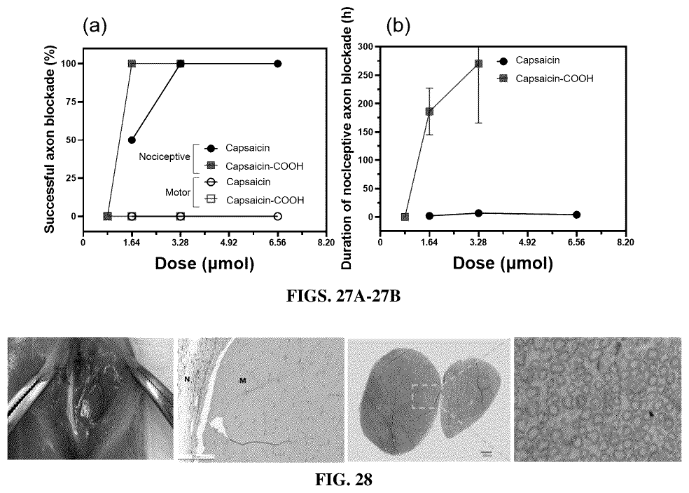

The study tested the in vivo efficacy and safety of the synthesized carboxylic acid derivative of capsaicin and pure unmodified capsaicin in blocking rat sciatic nerves. Adult male Sprague-Dawley rats weighing 250-350 g were injected with increasing doses of the carboxylic acid derivative of capsaicin or pure unmodified capsaicin (dissolved in 0.3 mL of 1×PBS buffer) peripherally at the left sciatic nerve. Neurobehavioral tests were performed to determine the duration of functional deficits, including nociceptive (measuring paw withdrawal latency using a hotplate test) and motor axon blockade (measuring the maximum weight a rat could bear using a weight-bearing test), in both hindpaws. Cohorts of rats that received injections of either the carboxylic acid derivative of capsaicin or pure unmodified capsaicin showed evidence of successful nociceptive axon blockade (i.e., thermal nociception blockade) in a dose-dependent manner with differences in the -duration of nerve block (FIGS. 27A-27B). Specifically, both groups achieved a 100% successful analgesic block (i.e., all rats tested showed thermal paw withdrawal latencies >7 seconds) at injection doses above 3.28 μmol. Notably, at a lower dose of 1.64 μmol, only the carboxylic acid derivative of capsaicin achieved this level of pain blockade (FIG. 27A). In addition, the injection did not produce any motor deficits (i.e., from motor fascicular blockade) in the injected hind limb. The study successfully induced a reversible nociceptive axon blockade following a single carboxylic acid derivative of capsaicin injection of 1.64 μmol and 3.28 μmol lasting for 186.0±35.5 and 270.0±90.4 hours, respectively (FIG. 27B). Importantly, the injection did not cause any capsaicin-related side effects, such as spontaneous pain behavior (e.g., flinching, licking), contralateral nociceptive axon blockade in the uninjected right hind limb, irreversible nociceptive axon blockade, seizure, or acute respiratory distress. In contrast, injection of 6.56 μmol pure unmodified capsaicin produced a nociceptive axon block lasting for 4.0±2.0 hours.

In order to study the local effects, the study euthanized treated rats 14 days after injection. The sciatic nerves and their surrounding tissues were harvested, sectioned, and stained for histologic evaluation. Hamstring muscles were processed and stained with hematoxylin-eosin (H&E). Epon-embedded semi-thin sciatic nerve sections and stained were generated and stained with toluidine blue (the gold-standard for peripheral nerve morphology). It was observed that the surrounding tissues in rats injected with 3.28 μmol of carboxylic acid derivative of capsaicin did not appear edematous or discolored, and there were no obvious signs of tissue injury (FIG. 28). Microscopic examination did not reveal significant myotoxicity or inflammation. Nerve sections stained with toluidine blue were normal in appearance without evidence of demyelination or axonal degeneration in any of the treated rats.

### Design, Synthesis, and Characterization of Carboxylic Acid Derivatives of Local Anesthetics

Design of local anesthetic derivatives: The study can synthesize 9 carboxylic acid derivatives of local anesthetics simultaneously by adding a carboxylic acid group to 9 clinically used local anesthetics (TABLE 4). The carboxylic acid group can be incorporated into the local anesthetics with either an ester bond or an amide bond. This can provide a comprehensive library of drugs to screen and identify the most suitable candidates for evaluation. In addition, the study can synthesize 2 control hydroxyl derivatives of local anesthetics, including hydroxyl group-functionalized bupivacaine and lidocaine, as the hydroxyl group may not utilize the MCT1 transporter to cross the PNBs.

TABLE 4
Chemical structures of derivatives of local anesthetics.

| Local anesthetics | Derivatives |
|---|---|
| 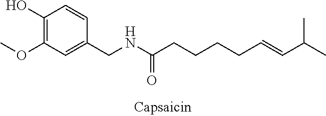 [SMILES: COc1cc(CNC(=O)CCCC/C=C/C(C)C)ccc1O] | 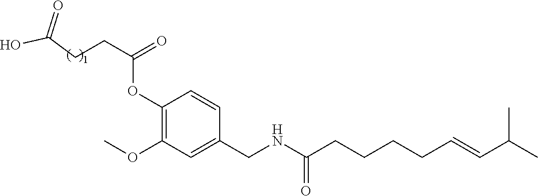 [SMILES: COc1cc(CNC(=O)CCCC/C=C/C(C)C)ccc1OC(=O)CCC(=O)O] |
| 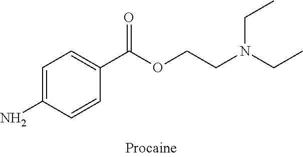 [SMILES: CCN(CC)CCOC(=O)c1ccc(N)cc1] | 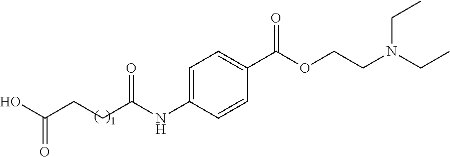 [SMILES: CCN(CC)CCOC(=O)c1ccc(NC(=O)CCC(=O)O)cc1] |
| 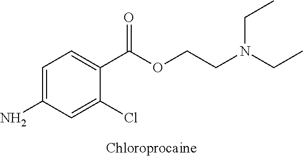 [SMILES: CCN(CC)CCOC(=O)c1ccc(N)cc1Cl] | 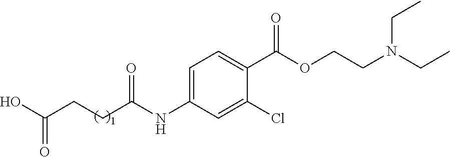 [SMILES: CCN(CC)CCOC(=O)c1ccc(NC(=O)CCC(=O)O)cc1Cl] |
| 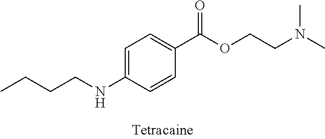 [SMILES: CCCCNc1ccc(C(=O)OCCN(C)C)cc1] | 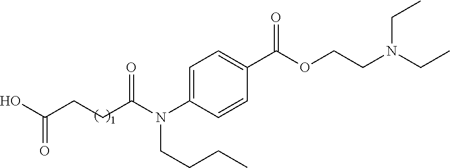 [SMILES: CCCCN(C(=O)CCC(=O)O)c1ccc(C(=O)OCCN(CC)CC)cc1] |
| 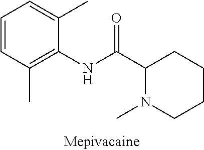 [SMILES: Cc1cccc(C)c1NC(=O)C1CCCCN1C] | 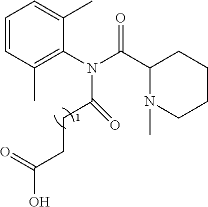 |
| 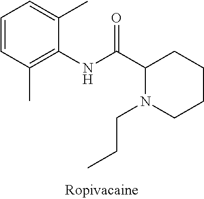 [SMILES: CCCN1CCCCC1C(=O)Nc1c(C)cccc1C] | 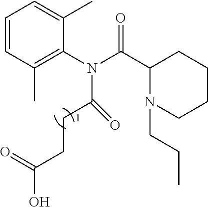 |
| 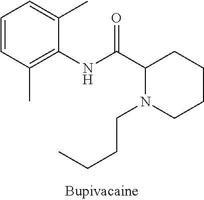 [SMILES: CCCCN1CCCCC1C(=O)Nc1c(C)cccc1C] | 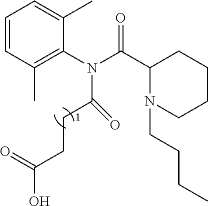 |
| 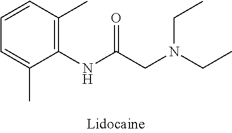 [SMILES: CCN(CC)CC(=O)Nc1c(C)cccc1C] | 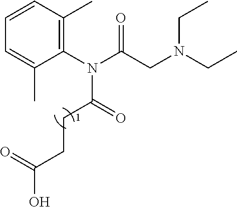 |
| 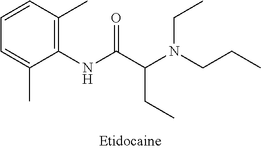 [SMILES: CCCN(CC)C(CC)C(=O)Nc1c(C)cccc1C] | 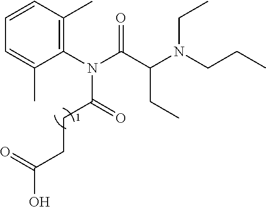 |

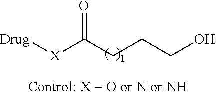

### Characterization of Local Anesthetic Derivatives:

The study can analyze the chemical structure and purity of synthesized local anesthetic derivatives using various analytical techniques, including ¹H NMR (Bruker 500), Fourier transform infrared spectrometer (FTIR, Alpha Bruker), MALDI-TOF, liquid chromatography-mass spectrometry (LC-MS), and UPLC. In addition, the study can determine basic drug properties, including:
i) Water solubility: This crucial parameter is essential for achieving the desired pharmacological response [29]. Inadequate water solubility often leads to low drug bioavailability. Moreover, water solubility is a key factor that guides injectable formulation development.
ii) Hydrolysis of local anesthetic derivatives: The study can explore the in vitro hydrolysis of local anesthetic derivatives in a pro-inflammatory pain microenvironment (e.g., acidic pH, and overexpressed enzymes). Specifically, the study can measure drug hydrolysis kinetics in 1×PBS with different pH values to elucidate the effect of pH on drug hydrolysis. The study can also measure drug hydrolysis kinetics in 1×PBS (pH 7.4) containing rat brain homogenate, which contains esterase enzymes, to evaluate the influence of enzymes on drug hydrolysis.
iii) Cytotoxicity: The study can perform validated cytotoxicity assays to determine drug myotoxicity and neurotoxicity potential.

### Experimental Methods

### Synthesis and Characterization of Local Anesthetic Derivatives:

The hydroxyl or amino groups of local anesthetics can be used as reaction sites for the incorporation of carboxylic acid groups. A preliminary study successfully synthesized the carboxylic acid derivative of capsaicin through the acylation reaction of capsaicin's active phenolic hydroxyl group with succinic anhydride [28]. In future research, the study can employ previously reported methods to synthesize carboxylic acid derivatives of ester local anesthetics and amide local anesthetics (SCHEME 5) [30-33]. To synthesize the control compounds, hydroxyl derivatives of local anesthetics, the study can convert the carboxylic acid group to the hydroxyl group using (Dimethyl sulfide) trihydroboron [34]. Finally, the chemical structure and purity of the synthesized compounds can be identified by NMR, FTIR, LC-MS, and UPLC.

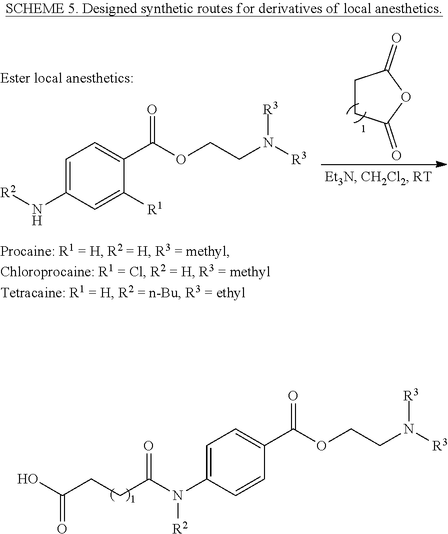

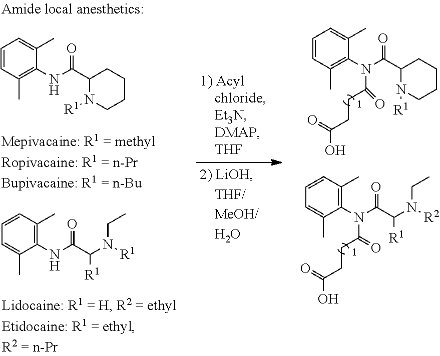

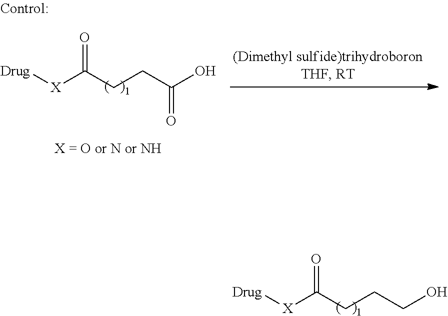

Water solubility and partition coefficients of drug in octanol/water. As previously reported [35], the study can determine the water solubility by sonicating excess drug in water for 15 minutes at room temperature, followed by centrifugation at 10,000 g for 2 minutes. The supernatant can be analyzed by UPLC and its partition coefficient (octanol/water) determined by vortexing the drug and octanol/water mixture for 10 minutes. The study can ascertain the drug concentrations in the octanol and water phases by UPLC. The drug partition coefficient can be calculated as follows: [c]_{octanol}/[c]_{water}.

### Fabrication of Injectable Formulations:

If the drug concentration to be tested is below the drug's water solubility, the drug can be dissolved directly in a 1×PBS buffer (pH 7.4). In cases where the drug concentration exceeds the drug's water solubility, Tween 20 can be used as a surfactant to generate a uniform drug suspension.

### Chemical Hydrolysis of Drugs:

The study can quantify the rate of drug chemical hydrolysis in 1×PBS at different pH and enzyme concentrations. Specifically, a predefined injectable drug amount can be added to 10 mL of 1×PBS at pH=6.0, 7.0, 7.4, and 7.8 or 1×PBS with rat brain homogenate (20% v/v) [36]. All solutions can be placed in a thermostatically controlled water bath at 37° C. At predefined intervals, the study can aliquot samples and analyze for remaining drug by UPLC and LC-MS. The study can calculate the pseudo-first-order half-time (t_{1/2}) for the drug hydrolysis from the slope of the linear portion of the remaining drug plotted logarithm against time.

### Cell Viability:

The study can use C2C12 mouse myoblasts, PC12 rat adrenal gland pheochromocytoma cells (a cell line frequently used in neurotoxicity studies [37, 38]), and rat DRG neurons [39] to evaluate the drug in vitro muscle cell and neuronal cell cytotoxicity, respectively. Briefly, cells can be incubated (1×10⁴ per well) with different drug concentrations (0.01, 0.1, 0.5, 1 mg/mL) for 24 hours in an incubator at 37° C. in 5% CO₂. After incubation, cells can be washed 5 times with warmed 1×PBS to remove any remaining drug, and cell viability can be determined by the 3-(4,5-dimethylthiazol-2-yl)-2,5-diphenyltetrazolium bromide (MTT) assay and live/dead assay, as previously published [40]. The study can administer unmodified local anesthetics as a positive control in this series of experiments.

### Drug Selection:

Carboxylic acid derivatives of local anesthetics with a confirmed chemical structure and purity exceeding 95% can be used for subsequent studies.

Screen to Select Carboxylic Acid Derivatives of Local Anesthetics with High Binding Affinity to the MCT1 Transporter

The study can investigate the binding affinities of 9 carboxylic acid derivatives of local anesthetics and 2 control hydroxyl derivatives of local anesthetics to MCT1 using an in situ rat brain perfusion technique. The study can select candidates with high MCT1 binding affinity for further investigation.

### Experimental Methods

### Animals:

The study can use adult (>70 day old) Sprague-Dawley rats weighing 225-350 grams. Several studies have shown that men and women respond differently to pain, with increased pain sensitivity and risk for chronic pain more commonly observed among women [41, 42]. To assess sex differences in response to local anesthetics as a biological variable, the study can directly compare age-matched female and male rats in the proposed experiments.

### In Situ Rat Brain Perfusion Technique:

The study can perform this assay as previously reported [36, 43-45]. Briefly, increasing drug concentrations can be co-perfused with [¹⁴C]benzoic acid (0.2 μCi/mL) into rat brains for 30 seconds. The study can determine the permeability-surface area product (PS) of [¹⁴C]benzoic acid after the perfusion. The study can evaluate the drug concentration dependent inhibition of [¹⁴C]benzoic acid uptake and its half maximal inhibitory concentration (IC₅₀) values deduced to compare MCT1 binding affinities.

### Drug Selection:

The study can select carboxylic acid derivatives of local anesthetics with measurable IC₅₀ values, indicating their ability to bind to MCT1, for subsequent studies.

Screen to Select Carboxylic Acid Derivatives of Local Anesthetics with High In Vitro BNB/Perineurium Permeability

MCT1 is highly expressed by endoneurial microvascular endothelial cells that form the human BNB and perineurium in vitro and in situ [22, 23][46]. In vitro solute permeability assays provide a high throughput avenue to study molecular permeability kinetics across endothelial and epithelial barrier systems. The study can determine whether selected carboxylic acid derivatives of local anesthetics undergo MCT1-mediated transport across an in vitro BNB and perineurium model to support enhanced permeability compared to unmodified local anesthetics.

### Experimental Methods

### In Vitro BNB Solute Permeability Assay:

The study can include static transwell insert solute permeability experiments using a simian virus long T antigen immortalized human endoneurial endothelial cell (THEndEC) line that retains essential characteristics of the human BNB, as previously published [47-49]. Briefly, 80,000 THEndECs can be seeded and grown to confluence on glutaraldehyde-crosslinked, rat tail collagen-coated 6.5 mm diameter Coring polyester membrane Transwell™ inserts (0.33 cm² surface area, 0.4 μm pore size) placed in 24-well tissue culture plates for 5 days in growth medium, and perform serum withdrawal for 2 days to inhibit growth prior to the permeability assays. The study can routinely monitor transendothelial electrical resistance (TEER) using a voltohmmeter (EVOM, World Precision Instruments) in concurrently plated inserts to ensure maximum expected TEER prior to permeability assays. The study can use collagen-coated Transwell inserts without cultured THEndECs as permeability assay controls. On day 7 after plating, Transwell inserts can be carefully transfered into freshly prepared 24 well plates containing 600 μL of 1% bovine serum albumin (BSA) in RPMI-1640, and 100 μL of predefined concentrations of selected carboxylic acid derivatives of local anesthetics or unmodified local anesthetics in 1% BSA in RPMI-1640 can be added into each insert in triplicate. The study can perform the solute permeability assays in a humidified incubator at 37° C. in 5% CO₂ for 6 hours. At predefined periods, the study can remove 24 well plates from the incubator, carefully remove and aspirate the solute from the Transwell insert into Eppendorf tubes while leaving solute in the 24-plate wells. The study can quantify solutes in the inserts and wells by HPLC and LC-MS, and determine solute permeability as a percentage of the input.

### In Vitro Perineurium Solute Permeability Assay:

The experimental methods are similar to those described above for the in vitro BNB solute permeability assay, with the exception of using a commercially available primary human perineurial cell line (e.g. ScienCell catalog #1710) instead of the THEndEC cell line.

### Drug Selection:

The study can compare carboxylic acid derivatives of local anesthetics in vitro BNB/perineurium permeability to unmodified local anesthetics. Candidates exhibiting statistically significant increases in solute permeability can be selected for in vivo peripheral nerve permeability testing.

Screen to Select Carboxylic Acid Derivatives of Local Anesthetics with High In Vivo Peripheral Nerve Penetration

Local anesthetics are administered around peripheral nerves and have to pass through tight junction forming PNBs, particularly the perineurium, into the endoneurium to inhibit axonal signal transduction. Previous studies show that perineurial cells highly express MCT1 [22-24]. The rat sciatic nerve model preliminary study shows that MCT1 facilitates peripherally injected carboxylic group-containing FITC permeability across PNBs into peripheral nerve endoneurium relative to FITC (FIGS. 26A-26B). The study can utilize this model to evaluate selected carboxylic acid derivatives of local anesthetics peripheral nerve permeability to determine whether the carboxylic acid moiety enhances drug peripheral nerve permeability in vivo. The study can select candidates with statistically significantly higher peripheral nerve permeability than their corresponding unmodified local anesthetics to study.

### Experimental Methods

Sciatic nerve injection: Under isoflurane-oxygen anesthesia, 1.0 mg of carboxylic acid derivatives of local anesthetics or their corresponding unmodified local anesthetics in 0.3 mL 1×PBS can be injected above the left sciatic nerve with a 23 G×¾″ needle. The needle can be introduced postero-medial to the greater trochanter, pointing in the anteromedial direction, and upon contact with bone the needle can be withdrawn slightly, and the formulation can be injected above the sciatic nerve.

### Drug Distribution:

Four hours after sciatic nerve injections, the study can perform carbon dioxide (CO₂) asphyxiation to euthanize rats and harvest the sciatic nerves from both sides (the sciatic nerve on the right side of the same rat can be used as the blank control). Sciatic nerve endoneurial drugs can be extracted as previously reported [50, 51]. The study can quantify drug amount by UPLC and LC-MS, respectively. The study can calculate drug peripheral nerve permeability as follows: solute permeability (%)=(amount of endoneurial drug/amount of drug administered)×100%. For each group, an equal number of male and female rats can be used for this test.

### Drug Selection:

The study can quantify and compare carboxylic acid derivatives of local anesthetics and their corresponding unmodified local anesthetics sciatic nerve endoneurial permeability. Since less than 1% of the unmodified local anesthetics injected perineurally cross the restrictive PNBs [15], the study can select carboxylic acid derivatives of local anesthetics that demonstrate an efficiency of >2% peripheral nerve permeability, supporting the notion that the carboxylic acid moiety enhances local anesthetic endoneurial transport, for further investigation as effective local anesthetics.

The study can determine success based on the following measurable milestones:
1) Obtain 9 carboxylic acid derivatives and 2 hydroxyl derivatives of local anesthetics with the designed structure and >95% purity.
2) Candidates have measurable IC₅₀ values for inhibiting [¹⁴C]benzoic acid uptake in the in situ rat brain perfusion experiment (i.e., carboxylic acid derivatives of local anesthetics have the ability to bind to MCT1).
3) Candidates exhibit statistically significant increases in solute permeability across in vitro BNB and perineurium models compared to pure unmodified local anesthetics.
4) Candidates that demonstrate an efficiency of >2% peripheral nerve permeability in an in vivo rat sciatic nerve model.

Additionally, the study could use a faster-hydrolyzed ester bond to connect the carboxylic acid group to local anesthetics.

### Sciatic Nerve Blockade in Normal Rats

The study can perineurally inject selected carboxylic acid derivatives of local anesthetics in the deep subcutaneous tissues over the left sciatic nerves in adult Sprague-Dawley rats (males: 250-350 g/females: 225-325 g). At predetermined time intervals, the study can perform a battery of validated neurobehavioral tests to evaluate sensory and motor functions in each rat. The study can also determine drug plasma pharmacokinetics (PK) profiles and pharmacodynamics (PD) concentrations concurrently.

### Sciatic Nerve Blockade:

The study can evaluate each rat for sensory and motor functional deficits following drug administration. These functional deficits indicate anesthesia, weakness or both involving the left hind limb below the knee. The study can ascertain the degree of sensory and motor nerve blockade using the thermal nociceptive Touch-Test sensory tests, and weight-bearing test, respectively.

Systemic Toxicity Associated with Local Anesthetics:

There are two principal clinical measures that indicate local anesthetic-induced systemic toxicity [1, 52]: i) sciatic nerve blockade in the un-injected (contralateral) limb suggests systemic drug distribution, and ii) clinical signs of distress. The study can use the same neurobehavioral tests described above to evaluate for sensory and motor nerve blockade in the right sciatic nerve. The study can closely observe rats for signs of distress, which include seizures, excessive salivation, staggering gait, irreversible nerve block, respiratory distress, and death [53]. The study can immediately euthanize rats that show agonal breathing or apneic spells.

The study can administer increasing doses of carboxylic acid derivatives of local anesthetics or pure unmodified local anesthetics and perform neurobehavioral assessments in different groups of rats until signs of systemic toxicity are observed. From these series of experiments, the study can determine duration of sensory and motor nerve blockade for each drug at different concentrations. The study can quantify toxicity as the dose per kg that caused the adverse effect in 50% of rats (i.e., the median effective dose (ED₅o)) for each of the toxicological endpoints.

Intravenous injection of a compound is generally assumed to be a bolus injection (i.e., it is delivered within a few seconds) [54, 55]. Since inadvertent intravascular injection can cause systemic anesthetic toxicity, the study can evaluate safety of intravenous drug administration.

### Study PK/PD Models:

The study can determine drug PK profiles and PD concentrations in parallel with sciatic nerve blockade and systemic toxicity assessments. At predetermined intervals, each rat can undergo sensory and motor neurobehavioral tests to evaluate for nerve blockade. The study can obtain blood from the tail immediately after completing neurobehavioral tests. Plasma drug concentration-time courses can be plotted to generate PK profiles. The study can define the lowest plasma drug concentration that causes signs of systemic toxicity as the drug toxic level in rats. The study can define the lowest plasma drug concentration that produces sciatic nerve block as the drug therapeutic level in rats. For each group, an equal number of male and female rats can be used for this test.

### Experimental Methods

### Sciatic Nerve Injection:

Under isoflurane-oxygen anesthesia, 0.3 mL of 1×PBS solution containing an increased dose of carboxylic acid derivatives of local anesthetics can be injected with a 23 G×¾″ needle posteromedial to the greater trochanter, pointing in an anteromedial direction in the deep subcutaneous tissue over the left sciatic nerve as previously described [40, 56]. Once bone is contacted, the needle can be slightly withdrawn, and 0.3 mL of the test solution containing an increased dose of drugs can be injected. The control rats can receive 0.3 mL test solution containing saline or pure unmodified local anesthetics.

### Intravenous Injection:

Under isoflurane-oxygen anesthesia, 0.3 mL of 1×PBS solution containing an increased dose of carboxylic acid derivatives of local anesthetics or pure unmodified local anesthetics can be injected via the tail vein using a 23 G×¾″ needle.

### Systemic Toxicity:

The study can observe rats for evidence of thermal paw withdrawal latency of 7 seconds in the un-injected (right) hind limb, seizures, excessive salivation, staggering gait, spontaneous pain behavior (e.g. flinching, licking), irreversible nerve block, respiratory distress, and death [53].

### Drug Determination in Plasma:

At predetermined intervals, the study can collect tail vein blood samples (0.1 mL) using a heparinized 1 mL disposable syringe after completing neurobehavioral tests in each rat. Multiple blood collections can be spaced far enough apart to prevent anemia, shock due to low blood volume, and distress in animals. Blood samples can be immediately mixed with an equal volume of modified Hank's balanced salt solution (pH 7.4) containing 500 units/mL heparin and centrifuge at 3000 g for 5 min. The resulting supernatant can be extracted with nine volumes of methanol containing 10% volume/volume acetic acid and ultrafiltered through a Vivaspin 500 (MWCO 5000, VivaScience AG). The filtrate can be lyophilized and redissolved in 20 mM heptafuluorobutylic acid in 10 mM ammonium formate (pH 4.0) for UPLC to determine drug concentrations.

### Sciatic Nerve Blockade in Rat Models of Inflammatory Pain and Traumatic Nerve Injury

The study aims to evaluate local anesthetic duration of action and safety in two animal models of pain. These studies aim to provide essential proof-of-principle data to guide future early phase clinical trials in chronic pain syndrome patients. The study can explore persistent paw inflammation as a model of chronic inflammatory pain [61] and spared sciatic nerve injury as a model of traumatic nerve injury [62]. Both models induce injury-induced hyperalgesia that persists for weeks to allow a conclusive determination of nerve blockade duration following drug administration.

The study can randomly assign cohorts of adult Sprague-Dawley male and female rats weighing 225-350 g to receive deep subcutaneous carboxylic acid derivatives of local anesthetics or pure unmodified local anesthetics or saline injection (as described above) after unilateral intraplantar Complete Freund's adjuvant (CFA) or spared sciatic nerve injury in the same hind limb 24 hours or 7 days afterward, respectively. The study can perform validated neurobehavioral assessments to evaluate local anesthetic efficacy on nerve blockade, and safety, as based on signs of local and systemic toxicity, compared to pure unmodified local anesthetics.

### Experimental Methods

### Drug Treatment:

0.3 mL of different concentrations of selected carboxylic acid derivatives of local anesthetics can be injected 24 hours after inducing paw inflammation or 7 days after spared sciatic nerve injury in the deep subcutaneous tissue over the left sciatic nerve with a 23 G×¾″ needle under brief isoflurane-oxygen anesthesia as previously described. The study can administer an equal volume of equivalent pure unmodified local anesthetics concentration (and normal saline) to age- and sex-matched controls, and evaluate sex as a biological variable in these series of experiments.

### Local Tissue and Organ Toxicity Studies

Biocompatibility is important because medical devices (or component materials) should not harm patients. The study can explore the local tissue and organ response to carboxylic acid derivatives of local anesthetics administration compared to pure unmodified local anesthetics.

### Local Tissue Toxicity:

Local tissue toxicity has been a major obstacle to developing long-duration local anesthetics [73, 74]. The study can explore the tissue reaction to carboxylic acid derivatives of local anesthetics. They can be histologically screened for signs of inflammation and myotoxicity and scored accordingly in a blinded manner. The study can perform light microscopy of toluidine blue-stained semi-thin sciatic nerve sections to detect axonal degeneration or demyelination following drug treatment in a blinded manner. The study can observe the axonal morphology and detect the diameter change of axons using transmission electron microscopy (TEM) in a blinded manner.

### Organ Toxicity:

One of the challenges in drug development is the potential for bioaccumulation in organs, leading to subsequent organ damage [75]. In particular, the liver is expected to be a major site of drug metabolism and liver toxicity of the drug should be monitored [76]. As previously reported [77], the heart, lung, spleen, liver, and kidneys can be histologically examined using standard techniques to study organ toxicity caused by carboxylic acid derivatives of local anesthetics.

### Experimental Methods

### Organ Tissue Abnormality [77]:

The study can generate blinded semi-quantitative histology scores to determine organ tissue abnormality as follows: 0. normal; 1. mild abnormality; 2. moderate abnormality; 3. severe pathological changes.

The study can determine success based on the following quantifiable milestones:
1) Ascertain the effectiveness and duration of nerve blockade produced by carboxylic acid derivatives of local anesthetics in normal male and female adult rats, as well as rats with inflammatory and traumatic nerve injury pain.
2) Determine therapeutic window of carboxylic acid derivatives of local anesthetics for nerve blockade without adverse effects based on PK/PD data.
3) Identify carboxylic acid derivatives of local anesthetics that prevent thermal nociception (defined as hind paw thermal withdrawal latency >7 seconds) in normal adult rats with a statistically significant longer duration compared to pure unmodified local anesthetics following a single sciatic nerve injection.
4) Identify carboxylic acid derivatives of local anesthetics that reverse reflexive nociception (thermal hyperalgesia, mechanical hypersensitivity and cold allodynia) from CFA-induced hind paw inflammation following a single injection 24 hours after inducing hind paw inflammation that persists for a statistically significant longer duration compared to pure unmodified local anesthetics.
5) Identify carboxylic acid derivatives of local anesthetics that reverse reflexive nociception (thermal hyperalgesia, mechanical hypersensitivity and cold allodynia) from sciatic nerve spared injury following a single injection 7 days after partial nerve branch ligation that persists for a statistically significant longer duration compared to pure unmodified local anesthetics.
6) Identify carboxylic acid derivatives of local anesthetics that do not cause adult rat biceps femoris muscle inflammation/toxicity and sciatic nerve toxicity and organ toxicity following deep subcutaneous administration based on defined histological scores.

The study can also synthesize additional derivatives of local anesthetics that can utilize other mechanisms to cross PNBs. One example is the lipid-conjugated local anesthetics, which may readily fuse with the lipid bilayers of cell membranes in PNBs, thereby facilitating the transportation of drugs across PNBs.

### Animal Methods

### In Situ Rat Brain Perfusion Technique:

The experiment can be carried out as previously reported [36, 43-45]. Briefly, rats can be anesthetized with pentobarbital (50 mg/kg, ip). After exposure of the right carotid artery system, the right external carotid artery can be ligated, the right common carotid artery can be cannulated with polyethylene (PE-50) catheters filled with 100 IU/mL heparin, and the right occipital and the right pterygopalatine arteries can be left open. Increasing concentrations of derivatives of local anesthetics with [¹⁴C]benzoic acid (0.2 μCi/mL) can be co-perfused into rat brain for 30 seconds. The permeability-surface area product (PS) of [¹⁴C]benzoic acid can be determined after the perfusion. The concentration dependence of derivatives of local anesthetics inhibiting [¹⁴C]benzoic acid uptake can be examined. The half maximal inhibitory concentration (IC₅₀) values of drugs can be measured to compare the binding affinity to MCT1.

### Sciatic Nerve Blockade in Normal Rats:

Prior to nerve block injections, the study can anesthetize rats briefly with inhalational isoflurane by facemask. A 23 G×¾″ needle can be introduced posteromedial to the left greater trochanter, pointing in an anteromedial direction. Once bone is contacted, the needle can be slightly withdrawn, and 0.3 mL of the test solution can be injected. The right side serves as an internal control, particularly for systemic toxicity studies. A sciatic nerve block is expected for 2-3 hours when injected accurately. The expected duration of general anesthesia is less than 2 minutes for sciatic nerve injections.

### Assessment of Nerve Block in Normal Rats:

Neurobehavioral testing can be done every 30 minutes for four testing periods, then hourly for four testing periods, then every six hours for the first 24 hours post-block, then twice per day until block resolves. The examiner can be blinded as to the contents of the injectate. The study can perform sensory function tests (hotplate test and Touch Test sensory test) and motor function tests by measuring extensor postural thrusts.

### Analysis of Neurobehavioral Data in Normal Rats:

The study can analyze neurobehavioral data to calculate the duration of nerve blockade. In all animals where injection does not result in a thermal withdrawal latency of at least 7 seconds, or at least a 50% reduction in weight-bearing in the extensor postural thrust test, the duration of effective block for the appropriate modality can be considered zero for computational purposes.

### Animal Models of Inflammatory Pain and Traumatic Nerve Injury:

Hind paw inflammation. The study can use complete Freund's adjuvant (CFA) that contains 0.1% heat-killed and dried Mycobacterium butyricum in 85% Marcol 52 and 15% Aracel A mannide monooleate emulsifier (Calbiochem, La Jolla, CA). A 0.15 mL intraplantar injection can be administered into the right hind paw after a brief isoflurane anesthesia [61]. The study can perform baseline neurobehavioral nociception tests and observe paw thickness prior to drug administration to evaluate efficacy and duration of action. The CFA can be used to induce the paw inflammation as it can induce hyperalgesia lasting approximately 1 to 2 weeks, a duration not achievable with other inflammatory agents such as mustard oil, carrageenan, and zymosan unless repeated injections are used [89].

Traumatic nerve injury: The study can anesthetize rats with inhalational isoflurane, place prone on a warmed platform maintained at 37° C. and the right hind limb cleaned and sterilized. The fur can be shaved, and the rat can be dissect through the skin just above the popliteal fossa. The hamstring muscles can be separated to expose the distao sciatic nerve and ligate the tibial and common peroneal nerves with nylon suture, leaving the sural nerve intact [62]. The incision can be closed with nylon suture and administer subcutaneous injection of lactated ringers. Inhalational anesthesia can then be discontinued, and rats can be allow to regain consciousness on a heated blanket/chamber maintained at 37° C. The study can perform validated reflexive neurobehavioral nociception tests before and after sciatic nerve spared nerve injury to establish baseline values and evaluate the efficacy and duration of drug blockade.

Drug Delivery and Neurobehavioral Reflexive Nociception Assessment in Rats with Primary Pain:

The study can perform drug delivery and neurobehavioral reflexive nociception assessment in rats with inflammatory pain and traumatic nerve injury.

### Intravenous Injection:

Under isoflurane-oxygen anesthesia, 0.3 mL of 1×PBS solution containing an increased dose of carboxylic acid derivatives of local anesthetics or pure unmodified local anesthetics can be injected via the tail vein using a 23 G×¾″ needle.

The following patents, applications and publications as listed below and throughout this document are hereby incorporated by reference in their entirety herein.

### Reference List for Example 1

[1] A. Gupta, K. Kaur, S. Sharma, S. Goyal, S. Arora, R. S. Murthy, Clinical aspects of acute post-operative pain management & its assessment, Journal of advanced pharmaceutical technology & research 1(2) (2010) 97-108.
[2] L. D. Brix, K. T. Bjørnholdt, T. M. Thillemann, L. Nikolajsen, Pain-related unscheduled contact with healthcare services after outpatient surgery, Anaesthesia 72(7) (2017) 870-878.
[3] G. Joshi, K. Gandhi, N. Shah, J. Gadsden, S. L. Corman, Peripheral nerve blocks in the management of postoperative pain: challenges and opportunities, Journal of Clinical Anesthesia 35 (2016) 524-529.
[4] C. B. Berde, U. Athiraman, B. Yahalom, D. Zurakowski, G. Corfas, C. Bognet, Tetrodotoxin-bupivacaineepinephrine combinations for prolonged local anesthesia, Mar Drugs 9(12) (2011) 2717-28.
[5] H. C. Dinges, T. Wiesmann, B. Otremba, H. Wulf, L. H. Eberhart, A. K. Schubert, The analgesic efficacy of liposomal bupivacaine compared with bupivacaine hydrochloride for the prevention of postoperative pain: a systematic review and meta-analysis with trial sequential analysis, Regional anesthesia and pain medicine 46(6) (2021) 490-498.
[6] B. M. Ilfeld, J. C. Eisenach, R. A. Gabriel, Clinical Effectiveness of Liposomal Bupivacaine Administered by Infiltration or Peripheral Nerve Block to Treat Postoperative Pain, Anesthesiology 134(2) (2021) 283-344.
[7] S. L. Orebaugh, A. Dewasurendra, Has the future arrived? Liposomal bupivacaine versus perineural catheters and additives for interscalene brachial plexus block, Current Opinion in Anesthesiology 33(5) (2020).
[8] Z. Wang, H. Huang, S. Yang, S. Huang, J. Guo, Q. Tang, F. Qi, Long-term effect of ropivacaine nanoparticles for sciatic nerve block on postoperative pain in rats, Int J Nanomedicine 11 (2016) 2081-90.
[9] K. Leasia, C. Ciarallo, J. T. H. Prins, C. Preslaski, E. Perkins-Pride, K. Hardin, A. Cralley, C. Cothren Burlew, J. J. Coleman, M. J. Cohen, R. Lawless, K. B. Platnick, E. E. Moore, F. M. Pieracci, A randomized clinical trial of single dose liposomal bupivacaine versus indwelling analgesic catheter in patients undergoing surgical stabilization of rib fractures, The journal of trauma and acute care surgery (2021).
[10] X. Zhou, J. Du, L. Qing, T. Mee, X. Xu, Z. Wang, C. Xu, X. Jia, Identification of sensory and motor nerve fascicles by immunofluorescence staining after peripheral nerve injury, Journal of Translational Medicine 19(1) (2021) 207.
[11] D. S. Kohane, S. E. Smith, D. N. Louis, G. Colombo, P. Ghoroghchian, N. G. M. Hunfeld, C. B. Berde, R. Langer, Prolonged duration local anesthesia from tetrodotoxin-enhanced local anesthetic microspheres, Pain 104(1) (2003) 415-421.
[12] M. Haanpää, R. D. Treede, Capsaicin for Neuropathic Pain: Linking Traditional Medicine and Molecular Biology, European Neurology 68(5) (2012) 264-275.
[13] A. M. Binshtok, B. P. Bean, C. J. Woolf, Inhibition of nociceptors by TRPV1-mediated entry of impermeant sodium channel blockers, Nature 449(7162) (2007) 607-10.
[14] P. Lirk, M. W. Hollmann, G. Strichartz, The Science of Local Anesthesia: Basic Research, Clinical Application, and Future Directions, Anesth Analg 126(4) (2018) 1381-1392.
[15] M. A. Ilie, C. Caruntu, M. Tampa, S. R. Georgescu, C. Matei, C. Negrei, R. M. Ion, C. Constantin, M. Neagu, D. Boda, Capsaicin: Physicochemical properties, cutaneous reactions and potential applications in painful and inflammatory conditions, Experimental and therapeutic medicine 18(2) (2019) 916-925.
[16] P. S. Chard, D. Bleakman, J. R. Savidge, R. J. Miller, Capsaicin-induced neurotoxicity in cultured dorsal root ganglion neurons: involvement of calcium-activated proteases, Neuroscience 65(4) (1995) 1099-108.
[17] T. Iwanaga, H. Takahashi-Iwanaga, J. Nio-Kobayashi, S. Ebara, Structure and barrier functions of the perineurium and its relationship with associated sensory corpuscles: A review, Biomedical research (Tokyo, Japan) 43(5) (2022) 145-159.
[18] S. H. Moghadam, E. Saliaj, S. D. Wettig, C. Dong, M. V. Ivanova, J. T. Huzil, M. Foldvari, Effect of chemical permeation enhancers on stratum corneum barrier lipid organizational structure and interferon alpha permeability, Mol Pharm 10(6) (2013) 2248-60.
[19] M. Mueckler, B. Thorens, The SLC2 (GLUT) family of membrane transporters, Molecular aspects of medicine 34(2-3) (2013) 121-38.
[20] C. F. Burant, G. I. Bell, Mammalian facilitative glucose transporters: Evidence for similar substrate recognition sites in functionally monomeric proteins, Biochemistry 31(42) (1992) 10414-10420.
[21] S. C. Rumsey, O. Kwon, G. W. Xu, C. F. Burant, I. Simpson, M. Levine, Glucose transporter isoforms GLUT1 and GLUT3 transport dehydroascorbic acid, J Biol Chem 272(30) (1997) 18982-9.
[22] Y. Sun, M. Zabihi, Q. Li, X. Li, B. J. Kim, E. E. Ubogu, S. N. Raja, U. Wesselmann, C. Zhao, Drug Permeability: From the Blood-Brain Barrier to the Peripheral Nerve Barriers, Advanced Therapeutics n/a(n/a) 2200150.
[23] C. M. Santamaria, C. Zhan, J. B. McAlvin, D. Zurakowski, D. S. Kohane, Tetrodotoxin, Epinephrine, and Chemical Permeation Enhancer Combinations in Peripheral Nerve Blockade, Anesth Analg 124(6) (2017) 1804-1812.
[24] S. G. Patching, Glucose Transporters at the Blood-Brain Barrier: Function, Regulation and Gateways for Drug Delivery, Mol Neurobiol 54(2) (2017) 1046-1077.
[25] G. Botti, A. Dalpiaz, B. Pavan, Targeting Systems to the Brain Obtained by Merging Prodrugs, Nanoparticles, and Nasal Administration, Pharmaceutics 13(8) (2021).
[26] J. Rautio, K. Laine, M. Gynther, J. Savolainen, Prodrug approaches for CNS delivery, The AAPS journal 10(1) (2008) 92-102.
[27] G. D. Holman, Structure, function and regulation of mammalian glucose transporters of the SLC2 family, Pflugers Archiv: European journal of physiology 472(9) (2020) 1155-1175.
[28] I. Tranoy-Opalinski, A. Fernandes, M. Thomas, J. P. Gesson, S. Papot, Design of self-immolative linkers for tumour-activated prodrug therapy, Anti-cancer agents in medicinal chemistry 8(6) (2008) 618-37.
[29] B. L. Kidd, L. A. Urban, Mechanisms of inflammatory pain, Br J Anaesth 87(1) (2001) 3-11.
[30] L. A. F. Law, K. A. Sluka, T. McMullen, J. Lee, L. Arendt-Nielsen, T. Graven-Nielsen, Acidic buffer induced muscle pain evokes referred pain and mechanical hyperalgesia in humans, Pain 140(2) (2008) 254-264.
[31] A. Nguyen, R. Bottger, S.-D. Li, Recent trends in bioresponsive linker technologies of Prodrug-Based Self-Assembling nanomaterials, Biomaterials 275 (2021) 120955.
[32] A. Rinaldi, R. Caraffi, M. V. Grazioli, N. Oddone, L. Giardino, G. Tosi, M. A. Vandelli, L. Calzà, B. Ruozi, J. T. Duskey, Applications of the ROS-Responsive Thioketal Linker for the Production of Smart Nanomedicines, Polymers (Basel) 14(4) (2022).
[33] K. T. Savjani, A. K. Gajjar, J. K. Savjani, Drug solubility: importance and enhancement techniques, ISRN pharmaceutics 2012 (2012) 195727.
[34] B. L. Kidd, L. A. Urban, Mechanisms of inflammatory pain, BJA: British Journal of Anaesthesia 87(1) (2001) 3-11.
[35] M. Mittal, M. R. Siddiqui, K. Tran, S. P. Reddy, A. B. Malik, Reactive oxygen species in inflammation and tissue injury, Antioxidants & redox signaling 20(7) (2014) 1126-67.
[36] Y. Lei, K. Wang, L. Deng, Y. Chen, E. C. Nice, C. Huang, Redox regulation of inflammation: old elements, a new story, Medicinal research reviews 35(2) (2015) 306-40.
[37] P. Wang, Q. Gong, J. Hu, X. Li, X. Zhang, Reactive Oxygen Species (ROS)-Responsive Prodrugs, Probes, and Theranostic Prodrugs: Applications in the ROS-Related Diseases, Journal of Medicinal Chemistry 64(1) (2021) 298-325.
[38] C. Skarbek, S. Serra, H. Maslah, E. Rascol, R. Labruere, Arylboronate prodrugs of doxorubicin as promising chemotherapy for pancreatic cancer, Bioorganic Chemistry 91 (2019) 103158.
[39] Y. Park, J. H. Park, S. Park, S. Y. Lee, K. H. Cho, D. D. Kim, W. S. Shim, I. S. Yoon, H. J. Cho, H. J. Maeng, Enhanced Cellular Uptake and Pharmacokinetic Characteristics of Doxorubicin-Valine Amide Prodrug, Molecules 21(10) (2016).
[40] X. Ma, E. Özliseli, Y. Zhang, G. Pan, D. Wang, H. Zhang, Fabrication of redox-responsive doxorubicin and paclitaxel prodrug nanoparticles with microfluidics for selective cancer therapy, Biomaterials Science 7(2) (2019) 634-644.
[41] B. Sun, C. Luo, X. Zhang, M. Guo, M. Sun, H. Yu, Q. Chen, W. Yang, M. Wang, S. Zuo, P. Chen, Q. Kan, H. Zhang, Y. Wang, Z. He, J. Sun, Probing the impact of sulfur/selenium/carbon linkages on prodrug nanoassemblies for cancer therapy, Nature Communications 10(1) (2019) 3211.
[42] Q. Pan, X. Deng, W. Gao, J. Chang, Y. Pu, B. He, ROS triggered cleavage of thioketal moiety to dissociate prodrug nanoparticles for chemotherapy, Colloids and surfaces. B, Biointerfaces 194 (2020) 111223.
[43] S. Guo, Y. Nakagawa, A. Barhoumi, W. Wang, C. Zhan, R. Tong, C. Santamaria, D. S. Kohane, Extended Release of Native Drug Conjugated in Polyketal Microparticles, Journal of the American Chemical Society 138(19) (2016) 6127-6130.
[44] A. Niethammer, G. Gaedicke, H. N. Lode, W. Wrasidlo, Synthesis and Preclinical Characterization of a Paclitaxel Prodrug with Improved Antitumor Activity and Water Solubility, Bioconjugate Chemistry 12(3) (2001) 414-420.
[45] D. A. Simone, T. K. Baumann, R. H. LaMotte, Dose-dependent pain and mechanical hyperalgesia in humans after intradermal injection of capsaicin, Pain 38(1) (1989) 99-107.
[46] M. Gynther, J. Ropponen, K. Laine, J. Leppanen, P. Haapakoski, L. Peura, T. Jarvinen, J. Rautio, Glucose promoiety enables glucose transporter mediated brain uptake of ketoprofen and indomethacin prodrugs in rats, J Med Chem 52(10) (2009) 3348-53.
[47] K. M. Mearow, M. E. Dodge, M. Rahimtula, C. Yegappan, Stress-mediated signaling in PC12 cells—the role of the small heat shock protein, Hsp27, and Akt in protecting cells from heat stress and nerve growth factor withdrawal, Journal of Neurochemistry 83(2) (2002) 452-462.
[48] H. Epstein-Barash, I. Shichor, A. H. Kwon, S. Hall, M. W. Lawlor, R. Langer, D. S. Kohane, Prolonged duration local anesthesia with minimal toxicity, Proc Natl Acad Sci USA 106(17) (2009) 7125-30.
[49] A. Mahmoodazdeh, S. M. Shafiee, M. Sisakht, Z. Khoshdel, M. A. Takhshid, Adrenomedullin protects rat dorsal root ganglion neurons against doxorubicin-induced toxicity by ameliorating oxidative stress, Iranian journal of basic medical sciences 23(9) (2020) 1197-1206.
[50] C. Zhao, A. Liu, C. M. Santamaria, A. Shomorony, T. Ji, T. Wei, A. Gordon, H. Elofsson, M. Mehta, R. Yang, D. S. Kohane, Polymer-tetrodotoxin conjugates to induce prolonged duration local anesthesia with minimal toxicity, Nat Commun 10(1) (2019) 2566-2566.
[51] E. J. Bartley, R. B. Fillingim, Sex differences in pain: a brief review of clinical and experimental findings, British journal of anaesthesia 111(1) (2013) 52-58.
[52] Z. Wiesenfeld-Hallin, Sex differences in pain perception, Gender Medicine 2(3) (2005) 137-145.
[53] Y. Takasato, S. I. Rapoport, Q. R. Smith, An in situ brain perfusion technique to study cerebrovascular transport in the rat, American Journal of Physiology-Heart and Circulatory Physiology 247(3) (1984) H484-H493.
[54] M. Gynther, K. Laine, J. Ropponen, J. Leppanen, A. Mannila, T. Nevalainen, J. Savolainen, T. Järvinen, J. Rautio, Large Neutral Amino Acid Transporter Enables Brain Drug Delivery via Prodrugs, Journal of Medicinal Chemistry 51(4) (2008) 932-936.
[55] N. Yosef, R. H. Xia, E. E. Ubogu, Development and characterization of a novel human in vitro blood-nerve barrier model using primary endoneurial endothelial cells, Journal of neuropathology and experimental neurology 69(1) (2010) 82-97.
[56] N. Yosef, E. E. Ubogu, An immortalized human blood-nerve barrier endothelial cell line for in vitro permeability studies, Cellular and molecular neurobiology 33(2) (2013) 175-86.
[57] S. P. Palladino, E. S. Helton, P. Jain, C. Dong, M. R. Crowley, D. K. Crossman, E. E. Ubogu, The Human Blood-Nerve Barrier Transcriptome, Scientific Reports 7(1) (2017) 17477.
[58] K. Devraj, M. E. Klinger, R. L. Myers, A. Mokashi, R. A. Hawkins, I. A. Simpson, GLUT-1 glucose transporters in the blood-brain barrier: differential phosphorylation, Journal of neuroscience research 89(12) (2011) 1913-25.
[59] S. G. Patching, Glucose Transporters at the Blood-Brain Barrier: Function, Regulation and Gateways for Drug Delivery, Molecular neurobiology 54(2) (2017) 1046-1077.
[60] P. Muona, S. Jaakkola, V. Salonen, J. Peltonen, Expression of glucose transporter 1 in adult and developing human peripheral nerve, Diabetologia 36(2) (1993) 133-40.
[61] A. R. Piña, M. M. Martínez, O. P. de Almeida, Glut-1, Best Immunohistochemical Marker for Perineurial Cells, Head and Neck Pathology 9(1) (2015) 104-106.
[62] H. Gao, S. Ho, J. Williams, LC-MS Bioanalysis of Drugs in Tissue Samples, Handbook of LC-MS Bioanalysis 2013, pp. 297-306.
[63] H. Gao, J. Williams, Tissue Sample Preparation in LC-MS Bioanalysis, Sample Preparation in LC-MS Bioanalysis 2019, pp. 201-216.
[64] Y. Sun, D. Zhao, G. Wang, Q. Jiang, M. Guo, Q. Kan, Z. He, J. Sun, A novel oral prodrug-targeting transporter MCT 1: 5-fluorouracil-dicarboxylate monoester conjugates, Asian J Pharm Sci 14(6) (2019) 631-639.
[65] A. Rawat, B. M. Morrison, Metabolic Transporters in the Peripheral Nerve-What, Where, and Why?, Neurotherapeutics: the journal of the American Society for Experimental NeuroTherapeutics 18(4) (2021) 2185-2199.
[66] D. S. Kohane, J. Yieh, N. T. Lu, R. Langer, G. R. Strichartz, C. B. Berde, A re-examination of tetrodotoxin for prolonged duration local anesthesia, Anesthesiology 89(1) (1998) 119-31.
[67] D. S. Kohane, W. N. Sankar, M. Shubina, D. Hu, N. Rifai, C. B. Berde, Sciatic nerve blockade in infant, adolescent, and adult rats: a comparison of ropivacaine with bupivacaine, Anesthesiology 89(5) (1998) 1199-1208.
[68] A. Saito, M. Yamamoto, Acute oral toxicity of capsaicin in mice and rats, The Journal of toxicological sciences 21(3) (1996) 195-200.
[69] K. H. Diehl, R. Hull, D. Morton, R. Pfister, Y. Rabemampianina, D. Smith, J. M. Vidal, C. van de Vorstenbosch, A good practice guide to the administration of substances and removal of blood, including routes and volumes, Journal of applied toxicology: JAT 21(1) (2001) 15-23.
[70] P. V. Turner, T. Brabb, C. Pekow, M. A. Vasbinder, Administration of substances to laboratory animals: routes of administration and factors to consider, J Am Assoc Lab Anim Sci 50(5) (2011) 600-613.
[71] B. Meibohm, H. Derendorf, Basic concepts of pharmacokinetic/pharmacodynamic (PK/PD) modelling, International journal of clinical pharmacology and therapeutics 35(10) (1997) 401-13.
[72] B. Hong, H. Chen, J. Han, Q. Xie, J. He, K. Bai, Y. Dong, R. Yi, A Study of 11-[³H]-Tetrodotoxin Absorption, Distribution, Metabolism and Excretion (ADME) in Adult Sprague-Dawley Rats, Mar Drugs 15(6) (2017) 159.
[73] V. Pratheepa, V. Vasconcelos, Binding and Pharmacokinetics of the Sodium Channel Blocking Toxins (Saxitoxin and the Tetrodotoxins), Mini reviews in medicinal chemistry 17(4) (2017) 320-327.
[74] Q. Li, X. Li, E. Bury, K. Lackey, A. Koh, U. Wesselmann, T. Yaksh, C. Zhao, Hydration-Induced Void-Containing Hydrogels for Encapsulation and Sustained Release of Small Hydrophilic Molecules, Advanced Functional Materials n/a(n/a) 2301025.
[75] V.r. Kayser, G.l. Guilbaud, The analgesic effects of morphine, but not those of the enkephalinase inhibitor thiorphan, are enhanced in arthritic rats, Brain Res. 267(1) (1983) 131-138.
[76] V. Kayser, G. Guilbaud, Differential effects of various doses of morphine and naloxone on two nociceptive test thresholds in arthritic and normal rats, Pain 41(3) (1990) 353-363.
[77] D. Fletcher, V. Kayser, G. Guilbaud, Influence of timing of administration on the analgesic effect of bupivacaine infiltration in carrageenin-injected rats, Anesthesiology 84(5) (1996) 1129-1137.
[78] M. A. Khan, P. Gerner, G. K. Wang, Amitriptyline for prolonged cutaneous analgesia in the rat, Anesthesiology 96(1) (2002) 109-116.
[79] C. Stein, M. J. Millan, A. Herz, Unilateral inflammation of the hindpaw in rats as a model of prolonged noxious stimulation: alterations in behavior and nociceptive thresholds, Pharmacology, biochemistry, and behavior 31(2) (1988) 445-51.
[80] I. Decosterd, C. J. Woolf, Spared nerve injury: an animal model of persistent peripheral neuropathic pain, Pain 87(2) (2000) 149-158.
[81] K. Hargreaves, R. Dubner, F. Brown, C. Flores, J. Joris, A new and sensitive method for measuring thermal nociception in cutaneous hyperalgesia, Pain 32(1) (1988) 77-88.
[82] R. P. Bonin, C. Bories, Y. De Koninck, A simplified up-down method (SUDO) for measuring mechanical nociception in rodents using von Frey filaments, Molecular pain 10 (2014) 26.
[83] D. S. Brenner, J. P. Golden, R. W.t. Gereau, A novel behavioral assay for measuring cold sensation in mice, PloS one 7(6) (2012) e39765.
[84] K. Sekimoto, M. Tobe, S. Saito, Local anesthetic toxicity: acute and chronic management, Acute medicine & surgery 4(2) (2017) 152-160.
[85] S. Yu, B. Wang, J. Zhang, K. Fang, The development of local anesthetics and their applications beyond anesthesia, 2019.
[86] S. Ritter, T. T. Dinh, Capsaicin-induced neuronal degeneration in the brain and retina of preweanling rats, The Journal of comparative neurology 296(3) (1990) 447-61.
[87] A. Shomorony, C. M. Santamaria, C. Zhao, A. Y. Rwei, M. Mehta, D. Zurakowski, D. S. Kohane, Prolonged Duration Local Anesthesia by Combined Delivery of Capsaicin- and Tetrodotoxin-Loaded Liposomes, Anesthesia & Analgesia 129(3) (2019).
[88] Q. Liu, C. M. Santamaria, T. Wei, C. Zhao, T. Ji, T. Yang, A. Shomorony, B. Y. Wang, D. S. Kohane, Hollow Silica Nanoparticles Penetrate the Peripheral Nerve and Enhance the Nerve Blockade from Tetrodotoxin, Nano Letters 18(1) (2018) 32-37.
[89] A. Shomorony, C. M. Santamaria, C. Zhao, A. Y. Rwei, M. Mehta, D. Zurakowski, D. S. Kohane, Prolonged Duration Local Anesthesia by Combined Delivery of Capsaicin- and Tetrodotoxin-Loaded Liposomes, Anesthesia & Analgesia 129(3) (2019) 709-717.
[80] W. Zhang, T. Ji, Y. Li, Y. Zheng, M. Mehta, C. Zhao, A. Liu, D. S. Kohane, Light-triggered release of conventional local anesthetics from a macromolecular prodrug for on-demand local anesthesia, Nature Communications 11(1) (2020) 2323.
[91] C. Weldon, T. Ji, M.-T. Nguyen, A. Rwei, W. Wang, Y. Hao, C. Zhao, M. Mehta, B. Y. Wang, J. Tsui, R. P. Marini, D. S. Kohane, Nanoscale Bupivacaine Formulations To Enhance the Duration and Safety of Intravenous Regional Anesthesia, ACS Nano 13(1) (2019) 18-25.
[92] X. Li, Q. Li, S. Song, A. O. Stevens, Z. Broemmel, Y. He, U. Wesselmann, T. Yaksh, C. Zhao, Emulsion-Induced Polymersomes Taming Tetrodotoxin for Prolonged Duration Local Anesthesia, Advanced Therapeutics n/a(n/a) (2022) 2200199.
[93] T. Ji, Y. Li, X. Deng, A. Y. Rwei, A. Offen, S. Hall, W. Zhang, C. Zhao, M. Mehta, D. S. Kohane, Delivery of local anaesthetics by a self-assembled supramolecular system mimicking their interactions with a sodium channel, Nature Biomedical Engineering (2021).
[94] M. Mehta, C. Zhao, A. Liu, C. Innocent, D. S. Kohane, Prolonged Retrobulbar Local Anesthesia of the Cornea Does Not Cause Keratopathy in Mice, Translational Vision Science & Technology 11(1) (2022) 33-33.
[95] S. Sakamoto, W. Putalun, S. Vimolmangkang, W. Phoolcharoen, Y. Shoyama, H. Tanaka, S. Morimoto, Enzyme-linked immunosorbent assay for the quantitative/qualitative analysis of plant secondary metabolites, J Nat Med 72(1) (2018) 32-42.
[96] A. Y. Rwei, J.-J. Lee, C. Zhan, Q. Liu, M. T. Ok, S. A. Shankarappa, R. Langer, D. S. Kohane, Repeatable and adjustable on-demand sciatic nerve block with phototriggerable liposomes, Proceedings of the National Academy of Sciences 112(51) (2015) 15719-15724.
[97] D. S. Kohane, R. Langer, Biocompatibility and drug delivery systems, Chemical Science 1(4) (2010) 441-446.
[98] F. Yuan, N. Yosef, C. Lakshmana Reddy, A. Huang, S. C. Chiang, H. R. Tithi, E. E. Ubogu, CCR2 gene deletion and pharmacologic blockade ameliorate a severe murine experimental autoimmune neuritis model of Guillain-Barré syndrome, PloS one 9(3) (2014) e90463.
[99] C. Dong, S. P. Palladino, E. S. Helton, E. E. Ubogu, The pathogenic relevance of a M-integrin in Guillain-Barré syndrome, Acta Neuropathologica 132(5) (2016) 739-752.
[100] N. Percie du Sert, V. Hurst, A. Ahluwalia, S. Alam, M. T. Avey, M. Baker, W. J. Browne, A. Clark, I. C. Cuthill, U. Dirnagl, M. Emerson, P. Garner, S. T. Holgate, D. W. Howells, N. A. Karp, S. E. Lazic, K. Lidster, C. J. MacCallum, M. Macleod, E. J. Pearl, O. H. Petersen, F. Rawle, P. Reynolds, K. Rooney, E. S. Sena, S. D. Silberberg, T. Steckler, H. WUrbel, The ARRIVE guidelines 2.0: Updated guidelines for reporting animal research, BMC Veterinary Research 16(1) (2020) 242.
[101] S. V. Frye, M. R. Arkin, C. H. Arrowsmith, P. J. Conn, M. A. Glicksman, E. A. Hull-Ryde, B. S. Slusher, Tackling reproducibility in academic preclinical drug discovery, Nature reviews. Drug discovery 14(11) (2015) 733-4.
[102] S. P. Jones, X.-L. Tang, Y. Guo, C. Steenbergen, D. J. Lefer, R. C. Kukreja, M. Kong, Q. Li, S. Bhushan, X. Zhu, J. Du, Y. Nong, H. L. Stowers, K. Kondo, G. N. Hunt, T. T. Goodchild, A. Orr, C. C. Chang, R. Ockaili, F. N. Salloum, R. Bolli, The NHLBI-Sponsored Consortium for preclinicAl assESsment of cARdioprotective Therapies (CAESAR), Circulation Research 116(4) (2015) 572-586.
[103] S. Perrin, Preclinical research: Make mouse studies work, Nature 507(7493) (2014) 423-425.
[104] Q. Li, X. Li, C. Zhao, Strategies to Obtain Encapsulation and Controlled Release of Small Hydrophilic Molecules, Front Bioeng Biotechnol 8 (2020) 437-437.
[105] K. Ren, R. Dubner, Inflammatory Models of Pain and Hyperalgesia, ILAR Journal 40(3) (1999) 111-118.

### Reference List for Example 2

[1] D. E. Becker, K. L. Reed, Anesthesia progress 2012, 59, 90.
[2] J. F. t. Butterworth, Reg Anesth Pain Med 2010, 35, 167.
[3] P. Lirk, M. W. Hollmann, G. Strichartz, Anesth. Analg. 2018, 126, 1381.
[4] H. Kehlet, T. S. Jensen, C. J. Woolf, Lancet 2006, 367, 1618.
[5] M. J. Caterina, M. A. Schumacher, M. Tominaga, T. A. Rosen, J. D. Levine, D. Julius, Nature 1997, 389, 816.
[6] A. E. Chávez, C. Q. Chiu, P. E. Castillo, Nat. Neurosci. 2010, 13, 1511.
[7] M. Tominaga, M. J. Caterina, A. B. Malmberg, T. A. Rosen, H. Gilbert, K. Skinner, B. E. Raumann, A. I. Basbaum, D. Julius, Neuron 1998, 21, 531.
[8] J. Zeng, Y. Lu, H. Chu, L. Lu, Y. Chen, K. Ji, Y. Lin, J. Li, S. Wang, Heliyon 2024.
[9] D. P. Mohapatra, C. Nau, Journal of Biological Chemistry 2003, 278, 50080.
[10] M. Haanpää, R. D. Treede, European Neurology 2012, 68, 264.
[11] A. M. Binshtok, B. P. Bean, C. J. Woolf, Nature 2007, 449, 607.
[12] S. Peltonen, M. Alanne, J. Peltonen, Tissue Barriers 2013, 1, e24956.
[13] P. Lirk, M. W. Hollmann, G. Strichartz, Anesth Analg 2018, 126, 1381.
[14] M. A. Ilie, C. Caruntu, M. Tampa, S. R. Georgescu, C. Matei, C. Negrei, R. M. Ion, C. Constantin, M. Neagu, D. Boda, Experimental and therapeutic medicine 2019, 18, 916.
[15] P. S. Chard, D. Bleakman, J. R. Savidge, R. J. Miller, Neuroscience 1995, 65, 1099.
[16] T. Knotts, K. Mease, L. Sangameswaran, M. Felx, S. Kramer, J. Donovan, J Orthop Res 2022, 40, 2281.
[17] Y. I. Asiri, M. Zaheen Hassan, Arabian Journal of Chemistry 2023, 16, 105180.
[18] Y. Lan, Y. Sun, T. Yang, X. Ma, M. Cao, L. Liu, S. Yu, A. Cao, Y. Liu, Molecular Pharmaceutics 2019, 16, 3430.
[19] L. Linderoth, G. H. Peters, R. Madsen, T. L. Andresen, Angew. Chem. Int. Ed. 2009, 48, 1823.
[20] Y. Feng, Y. Zhu, J. Wan, X. Yang, C. K. Firempong, J. Yu, X. Xu, Journal of Functional Foods 2018, 44, 137.
[21] A. Shomorony, C. M. Santamaria, C. Zhao, A. Y. Rwei, M. Mehta, D. Zurakowski, D. S. Kohane, Anesthesia & Analgesia 2019, 129, 709.
[22] A. J. Cura, A. Carruthers, Compr Physiol 2012, 2, 863.
[23] T. Iwanaga, H. Takahashi-Iwanaga, J. Nio-Kobayashi, S. Ebara, Biomedical research (Tokyo, Japan) 2022, 43, 145.
[24] S. H. Moghadam, E. Saliaj, S. D. Wettig, C. Dong, M. V. Ivanova, J. T. Huzil, M. Foldvari, Mol Pharm 2013, 10, 2248.
[25] P. Magnani, P. V. Cherian, G. W. Gould, D. A. Greene, A. A. Sima, F. C. Brosius, 3rd, Metabolism 1996, 45, 1466.
[26] M. Mueckler, B. Thorens, Molecular aspects of medicine 2013, 34, 121.
[27] C. F. Burant, G. I. Bell, Biochemistry 1992, 31, 10414.
[28] S. C. Rumsey, O. Kwon, G. W. Xu, C. F. Burant, I. Simpson, M. Levine, J Biol Chem 1997, 272, 18982.
[29] Y. Sun, M. Zabihi, Q. Li, X. Li, B. J. Kim, E. E. Ubogu, S. N. Raja, U. Wesselmann, C. Zhao, Advanced Therapeutics n/a, 2200150.
[30] B. Thorens, M. Mueckler, Am J Physiol Endocrinol Metab 2010, 298, E141.
[31] A. Dalpiaz, R. Filosa, P. de Caprariis, G. Conte, F. Bortolotti, C. Biondi, A. Scatturin, P. D. Prasad, B. Pavan, Int. J. Pharm. 2007, 336, 133.
[32] M. Gynther, J. Ropponen, K. Laine, J. Leppanen, P. Haapakoski, L. Peura, T. Jarvinen, J. Rautio, J. Med. Chem. 2009, 52, 3348.
[33] K. Mikuni, K. Nakanishi, K. Hara, K. Hara, W. Iwatani, T. Amano, K. Nakamura, Y. Tsuchiya, H. Okumoto, T. Mandai, Biol Pharm Bull 2008, 31, 1155.
[34] D. Melisi, A. Curcio, E. Luongo, E. Morelli, M. G. Rimoli, Curr. Top. Med. Chem. 2011, 11, 2288.
[35] A. Shomorony, C. M. Santamaria, C. Zhao, A. Y. Rwei, M. Mehta, D. Zurakowski, D. S. Kohane, Anesth Analg 2019, 129, 709.
[36] A. Y. Rwei, J. J. Lee, C. Zhan, Q. Liu, M. T. Ok, S. A. Shankarappa, R. Langer, D. S. Kohane, Proc. Nat. Acad. Sci. U.S.A. 2015, 112, 15719.
[37] Y. Li, T. Ji, M. Torre, R. Shao, Y. Zheng, D. Wang, X. Li, A. Liu, W. Zhang, X. Deng, R. Yan, D. S. Kohane, Nat. Commun. 2023, 14, 6659.
[38] T. Ji, Y. Li, X. Deng, A. Y. Rwei, A. Offen, S. Hall, W. Zhang, C. Zhao, M. Mehta, D. S. Kohane, Nat Biomed Eng 2021, 5, 1099.
[39] E. J. Simons, E. Bellas, M. W. Lawlor, D. S. Kohane, Molecular pharmaceutics 2009, 6, 265.
[40] D. A. Simone, M. Nolano, T. Johnson, G. Wendelschafer-Crabb, W. R. Kennedy, J. Neurosci. 1998, 18, 8947.
[41] M. J. M. Fischer, C. I. Ciotu, A. Szallasi, Frontiers in Physiology 2020, 11.
[42] M. Gynther, J. Ropponen, K. Laine, J. Leppanen, P. Haapakoski, L. Peura, T. Jarvinen, J. Rautio, J. Med. Chem. 2009, 52, 3348.
[43] K. Olszewski, A. Barsotti, X. J. Feng, M. Momcilovic, K. G. Liu, J. I. Kim, K. Morris, C. Lamarque, J. Gaffney, X. Yu, J. P. Patel, J. D. Rabinowitz, D. B. Shackelford, M. V. Poyurovsky, Cell Chem Biol 2022, 29, 423.
[44] J. D. Christensen, S. L. Vecchio, H. H. Andersen, J. Elberling, L. Arendt-Nielsen, The Journal of Pain 2021, 22, 778.
[45] A. M. Binshtok, P. Gerner, S. B. Oh, M. Puopolo, S. Suzuki, D. P. Roberson, T. Herbert, C. F. Wang, D. Kim, G. Chung, A. A. Mitani, G. K. Wang, B. P. Bean, C. J. Woolf, Anesthesiology 2009, 111, 127.
[46] G. Botti, A. Dalpiaz, B. Pavan, Pharmaceutics 2021, 13.
[47] J. Rautio, K. Laine, M. Gynther, J. Savolainen, The AAPS journal 2008, 10, 92.
[48] A. Dalpiaz, R. Filosa, P. de Caprariis, G. Conte, F. Bortolotti, C. Biondi, A. Scatturin, P. D. Prasad, B. Pavan, Int J Pharm 2007, 336, 133.
[49] C. Fernández, O. Nieto, J. A. Fontenla, E. Rivas, M. L. de Ceballos, A. Fernindez-Mayoralas, Org. Biomol. Chem. 2003, 1, 767.
[50] T. Halmos, M. Santarromana, K. Antonakis, D. Scherman, Eur. J. Pharmacol. 1996, 318, 477.
[51] Y.-S. Lin, R. Tungpradit, S. Sinchaikul, F.-M. An, D.-Z. Liu, S. Phutrakul, S.-T. Chen, J. Med. Chem. 2008, 51, 7428.
[52] C. M. Santamaria, C. Zhan, J. B. McAlvin, D. Zurakowski, D. S. Kohane, Anesth Analg 2017, 124, 1804.
[53] H. Manjunatha, K. Srinivasan, Can. J. Physiol. Pharmacol. 2007, 85, 588.
[54] E. Marini, G. Magi, M. Mingoia, A. Pugnaloni, B. Facinelli, Frontiers in Microbiology 2015, 6.
[55] C.-S. Kim, T. Kawada, B.-S. Kim, I.-S. Han, S.-Y. Choe, T. Kurata, R. Yu, Cell. Signal. 2003, 15, 299.
[56] J. R. Friedman, N. A. Nolan, K. C. Brown, S. L. Miles, A. T. Akers, K. W. Colclough, J. M. Seidler, J. M. Rimoldi, M. A. Valentovic, P. Dasgupta, J. Pharmacol. Exp. Ther. 2018, 364, 462.
[57] J. Zheng, S. Zheng, Q. Feng, Q. Zhang, X. Xiao, Biosci. Rep. 2017, 37.
[58] M. Lu, C. Chen, Y. Lan, J. Xiao, R. Li, J. Huang, Q. Huang, Y. Cao, C. T. Ho, Food Funct 2020, 11, 2848.
[59] Y. Sun, D. Zhao, G. Wang, Q. Jiang, M. Guo, Q. Kan, Z. He, J. Sun, Asian J Pharm Sci 2019, 14, 631.
[60] A. Rawat, B. M. Morrison, Neurotherapeutics: the journal of the American Society for Experimental NeuroTherapeutics 2021, 18, 2185.
[61] Q. Liu, C. M. Santamaria, T. Wei, C. Zhao, T. Ji, T. Yang, A. Shomorony, B. Y. Wang, D. S. Kohane, Nano Lett. 2018, 18, 32.
[62] J. B. McAlvin, R. F. Padera, S. A. Shankarappa, G. Reznor, A. H. Kwon, H. H. Chiang, J. S. Yang, D. S. Kohane, Biomaterials 2014, 35, 4557.

### Reference List for Example 4

[1] D. S. Kohane, W. N. Sankar, M. Shubina, D. Hu, N. Rifai, C. B. Berde, Sciatic nerve blockade in infant, adolescent, and adult rats: a comparison of ropivacaine with bupivacaine, Anesthesiology 89(5) (1998) 1199-208.
[2] D. S. Kohane, M. Lipp, R. C. Kinney, N. Lotan, R. Langer, Sciatic Nerve Blockade with Lipid-Protein-Sugar Particles Containing Bupivacaine, Pharmaceutical Research 17(10) (2000) 1243-1249.
[3] D. S. Kohane, C. B. Berde, G. Strichartz, R. S. Langer, Local anesthetic formulations, Google Patents, 2001.
[4] G. Joshi, K. Gandhi, N. Shah, J. Gadsden, S. L. Corman, Peripheral nerve blocks in the management of postoperative pain: challenges and opportunities, Journal of Clinical Anesthesia 35 (2016) 524-529.
[5] C. B. Berde, U. Athiraman, B. Yahalom, D. Zurakowski, G. Corfas, C. Bognet, Tetrodotoxin-bupivacaine-epinephrine combinations for prolonged local anesthesia, Mar Drugs 9(12) (2011) 2717-28.
[6] H. C. Dinges, T. Wiesmann, B. Otremba, H. Wulf, L. H. Eberhart, A. K. Schubert, The analgesic efficacy of liposomal bupivacaine compared with bupivacaine hydrochloride for the prevention of postoperative pain: a systematic review and meta-analysis with trial sequential analysis, Regional anesthesia and pain medicine 46(6) (2021) 490-498.
[7] B. M. Ilfeld, J. C. Eisenach, R. A. Gabriel, Clinical Effectiveness of Liposomal Bupivacaine Administered by Infiltration or Peripheral Nerve Block to Treat Postoperative Pain, Anesthesiology 134(2) (2021) 283-344.
[8] S. L. Orebaugh, A. Dewasurendra, Has the future arrived? Liposomal bupivacaine versus perineural catheters and additives for interscalene brachial plexus block, Current Opinion in Anesthesiology 33(5) (2020).
[9] Z. Wang, H. Huang, S. Yang, S. Huang, J. Guo, Q. Tang, F. Qi, Long-term effect of ropivacaine nanoparticles for sciatic nerve block on postoperative pain in rats, Int J Nanomedicine 11 (2016) 2081-90.
[10] K. Leasia, C. Ciarallo, J. T. H. Prins, C. Preslaski, E. Perkins-Pride, K. Hardin, A. Cralley, C. Cothren Burlew, J. J. Coleman, M. J. Cohen, R. Lawless, K. B. Platnick, E. E. Moore, F. M. Pieracci, A randomized clinical trial of single dose liposomal bupivacaine versus indwelling analgesic catheter in patients undergoing surgical stabilization of rib fractures, The journal of trauma and acute care surgery (2021).
[11] R. Padera, E. Bellas, J. Y. Tse, D. Hao, D. S. Kohane, Local myotoxicity from sustained release of bupivacaine from microparticles, Anesthesiology 108(5) (2008) 921-928.
[12] P. PERE, T. WAHLSTROM, H. WATANABE, P. H. ROSENBERG, M. PITKANEN, Local Myotoxicity of Bupivacaine in Rabbits After Continuous Supraclavicular Brachial Plexus Block, Regional Anesthesia and Pain Medicine 18(5) (1993) 304-307.
[13] P. S. Chard, D. Bleakman, J. R. Savidge, R. J. Miller, Capsaicin-induced neurotoxicity in cultured dorsal root ganglion neurons: involvement of calcium-activated proteases, Neuroscience 65(4) (1995) 1099-108.
[14] R. A. Gabriel, B. M. Ilfeld, Peripheral nerve blocks for postoperative analgesia: From traditional unencapsulated local anesthetic to liposomes, cryoneurolysis and peripheral nerve stimulation, Best practice & research. Clinical anaesthesiology 33(3) (2019) 293-302.
[15] P. Lirk, M. W. Hollmann, G. Strichartz, The Science of Local Anesthesia: Basic Research, Clinical Application, and Future Directions, Anesth Analg 126(4) (2018) 1381-1392.
[16] Y. Sun, M. Zabihi, Q. Li, X. Li, B. J. Kim, E. E. Ubogu, S. N. Raja, U. Wesselmann, C. Zhao, Drug Permeability: From the Blood-Brain Barrier to the Peripheral Nerve Barriers, Advanced Therapeutics 6(4) (2023) 2200150.
[17] N. Vijay, M. E. Morris, Role of monocarboxylate transporters in drug delivery to the brain, Current pharmaceutical design 20(10) (2014) 1487-98.
[18] V. N. Jackson, A. P. Halestrap, The kinetics, substrate, and inhibitor specificity of the monocarboxylate (lactate) transporter of rat liver cells determined using the fluorescent intracellular pH indicator, 2′,7′-bis(carboxyethyl)-5(6)-carboxyfluorescein, J Biol Chem 271(2) (1996) 861-8.
[19] A. P. Halestrap, D. Meredith, The SLC16 gene family-from monocarboxylate transporters (MCTs) to aromatic amino acid transporters and beyond, Pflugers Archiv: European journal of physiology 447(5) (2004) 619-28.
[20] G. Li, K. Shao, C. S. Umeshappa, 3—Recent progress in blood-brain barrier transportation research, in: H. Gao, X. Gao (Eds.), Brain Targeted Drug Delivery System, Academic Press2019, pp. 33-51.
[21] Y. Kang, T. Terasaki, A. Tsuji, ACIDIC DRUG TRANSPORT I N VIVO THROUGH THE BLOOD-BRAIN BARRIER. A ROLE O F THE TRANSPORT CARRIER FOR MONOCARBOXYLIC ACIDS, Journal of Pharmacobio-Dynamics 13(2) (1990) 158-163.
[22] A. Rawat, B. M. Morrison, Metabolic Transporters in the Peripheral Nerve-What, Where, and Why?, Neurotherapeutics: the journal of the American Society for Experimental NeuroTherapeutics 18(4) (2021) 2185-2199.
[23] B. Morrison, A. Tsingalia, S. Lengacher, L. Pellerin, P. Magistretti, J. Rothstein, Monocarboxylate Transporter 1 (MCT1) is Critical for Regeneration in Peripheral Nerves (I6-1.001), Neurology 82(10 Supplement) (2014) I6-1.001.
[24] M. K. Jha, X. H. Ament, F. Yang, Y. Liu, M. J. Polydefkis, L. Pellerin, B. M. Morrison, Reducing monocarboxylate transporter MCT1 worsens experimental diabetic peripheral neuropathy, Experimental neurology 333 (2020) 113415.
[25] B. M. Morrison, A. Tsingalia, S. Vidensky, Y. Lee, L. Jin, M. H. Farah, S. Lengacher, P. J. Magistretti, L. Pellerin, J. D. Rothstein, Deficiency in monocarboxylate transporter 1 (MCT1) in mice delays regeneration of peripheral nerves following sciatic nerve crush, Experimental neurology 263 (2015) 325-38.
[26] S. H. Moghadam, E. Saliaj, S. D. Wettig, C. Dong, M. V. Ivanova, J. T. Huzil, M. Foldvari, Effect of chemical permeation enhancers on stratum corneum barrier lipid organizational structure and interferon alpha permeability, Mol Pharm 10(6) (2013) 2248-60.
[27] C. M. Santamaria, C. Zhan, J. B. McAlvin, D. Zurakowski, D. S. Kohane, Tetrodotoxin, Epinephrine, and Chemical Permeation Enhancer Combinations in Peripheral Nerve Blockade, Anesth Analg 124(6) (2017) 1804-1812.
[28] H. Shin, S. Kim, J. Kim, S. Kong, Y. Lee, J.-C. Lee, Preparation of 3-pentadecylphenol-modified cellulose nanocrystal and its application as a filler to polypropylene nanocomposites having improved antibacterial and mechanical properties, Journal of Applied Polymer Science 139(13) (2022) 51848.
[29] K. T. Savjani, A. K. Gajjar, J. K. Savjani, Drug solubility: importance and enhancement techniques, ISRN pharmaceutics 2012 (2012) 195727.
[30] J. Xu, Z. Yang, J. Hua, Y. Lin, M. Bian, Y. Li, C. Liu, W. He, Z. Fang, K. Guo, The continuous-flow electrosynthesis of 4-(sulfonylmethyl)isoquinoline-1,3(2H,4H)-diones from N-alkyl-N-methacryloyl benzamides under metal-free and oxidant-free conditions, Organic Chemistry Frontiers 7(20) (2020) 3223-3228.
[31] X. Cai, Y. Liu, S. Ding, J. Fu, J. Li, D. Cheng, X. Xu, Visible light-induced radical cascade reaction of acryloylbenzamides with N-hydroxyphthalimide esters, Tetrahedron Letters 95 (2022) 153722.
[32] J. Fu, X. Cai, Y. Liu, J.-H. Li, D. Cheng, X. xu, Synthesis of Isoquinoline-1,3(2H,4H)-diones by Visible-light-mediated the Cyclization of Acryloylbenzamides with Alkyl Boronic Acids, Arylsulfonylhydrazides and Oxime Esters, Synthesis 0 (2022).
[33] Y. Ma, X. Yang, H. Han, Z. Wen, M. Yang, Y. Zhang, J. Fu, X. Wang, T. Yin, G.-H. Lu, J. qi, H. Lin, X. Wang, Y. Yang, Design, synthesis and biological evaluation of anilide (dicarboxylic acid) shikonin esters as antitumor agents through targeting PI3K/Akt/mTOR signaling pathway, Bioorganic Chemistry 111 (2021) 104872.
[34] C. V. Michael, O. Oswald, Borane-Methyl Sulfide Reductive Cyclization of o-Ester Alkylamides: A Convenient Synthesis of <i>N</i>-Substituted Cyclic Amines, Synthesis 1988(12) (1988) 985-988.
[35] A. Niethammer, G. Gaedicke, H. N. Lode, W. Wrasidlo, Synthesis and Preclinical Characterization of a Paclitaxel Prodrug with Improved Antitumor Activity and Water Solubility, Bioconjugate Chemistry 12(3) (2001) 414-420.
[36] M. Gynther, J. Ropponen, K. Laine, J. Leppanen, P. Haapakoski, L. Peura, T. Jarvinen, J. Rautio, Glucose promoiety enables glucose transporter mediated brain uptake of ketoprofen and indomethacin prodrugs in rats, J Med Chem 52(10) (2009) 3348-53.
[37] K. M. Mearow, M. E. Dodge, M. Rahimtula, C. Yegappan, Stress-mediated signaling in PC12 cells—the role of the small heat shock protein, Hsp27, and Akt in protecting cells from heat stress and nerve growth factor withdrawal, Journal of Neurochemistry 83(2) (2002) 452-462.
[38] H. Epstein-Barash, I. Shichor, A. H. Kwon, S. Hall, M. W. Lawlor, R. Langer, D. S. Kohane, Prolonged duration local anesthesia with minimal toxicity, Proc Natl Acad Sci USA 106(17) (2009) 7125-30.
[39] A. Mahmoodazdeh, S. M. Shafiee, M. Sisakht, Z. Khoshdel, M. A. Takhshid, Adrenomedullin protects rat dorsal root ganglion neurons against doxorubicin-induced toxicity by ameliorating oxidative stress, Iranian journal of basic medical sciences 23(9) (2020) 1197-1206.
[40] C. Zhao, A. Liu, C. M. Santamaria, A. Shomorony, T. Ji, T. Wei, A. Gordon, H. Elofsson, M. Mehta, R. Yang, D. S. Kohane, Polymer-tetrodotoxin conjugates to induce prolonged duration local anesthesia with minimal toxicity, Nature Communications 10(1) (2019) 2566.
[41] E. J. Bartley, R. B. Fillingim, Sex differences in pain: a brief review of clinical and experimental findings, British journal of anaesthesia 111(1) (2013) 52-58.
[42] Z. Wiesenfeld-Hallin, Sex differences in pain perception, Gender Medicine 2(3) (2005) 137-145.
[43] Y. Takasato, S. I. Rapoport, Q. R. Smith, An in situ brain perfusion technique to study cerebrovascular transport in the rat, American Journal of Physiology-Heart and Circulatory Physiology 247(3) (1984) H484-H493.
[44] M. Gynther, K. Laine, J. Ropponen, J. Leppanen, A. Mannila, T. Nevalainen, J. Savolainen, T. Järvinen, J. Rautio, Large Neutral Amino Acid Transporter Enables Brain Drug Delivery via Prodrugs, Journal of Medicinal Chemistry 51(4) (2008) 932-936.
[45] Y. Kido, I. Tamai, M. Okamoto, F. Suzuki, A. Tsuji, Functional Clarification of MCT1-Mediated Transport of Monocarboxylic Acids at the Blood-Brain Barrier Using In vitro Cultured Cells and In vivo BUI Studies, Pharmaceutical Research 17(1) (2000) 55-62.
[46] M. K. Jha, X. H. Ament, F. Yang, Y. Liu, M. J. Polydefkis, L. Pellerin, B. M. Morrison, Reducing monocarboxylate transporter MCT1 worsens experimental diabetic peripheral neuropathy, bioRxiv (2020) 2020.02.26.965152.
[47] N. Yosef, R. H. Xia, E. E. Ubogu, Development and characterization of a novel human in vitro blood-nerve barrier model using primary endoneurial endothelial cells, Journal of neuropathology and experimental neurology 69(1) (2010) 82-97.
[48] N. Yosef, E. E. Ubogu, An immortalized human blood-nerve barrier endothelial cell line for in vitro permeability studies, Cellular and molecular neurobiology 33(2) (2013) 175-86.
[49] S. P. Palladino, E. S. Helton, P. Jain, C. Dong, M. R. Crowley, D. K. Crossman, E. E. Ubogu, The Human Blood-Nerve Barrier Transcriptome, Scientific Reports 7(1) (2017) 17477.
[50] H. Gao, S. Ho, J. Williams, LC-MS Bioanalysis of Drugs in Tissue Samples, Handbook of LC—MS Bioanalysis 2013, pp. 297-306.
[51] H. Gao, J. Williams, Tissue Sample Preparation in LC-MS Bioanalysis, Sample Preparation in LC-MS Bioanalysis 2019, pp. 201-216.
[52] D. S. Kohane, J. Yieh, N. T. Lu, R. Langer, G. R. Strichartz, C. B. Berde, A re-examination of tetrodotoxin for prolonged duration local anesthesia, Anesthesiology 89(1) (1998) 119-31.
[53] A. Saito, M. Yamamoto, Acute oral toxicity of capsaicin in mice and rats, The Journal of toxicological sciences 21(3) (1996) 195-200.
[54] K. H. Diehl, R. Hull, D. Morton, R. Pfister, Y. Rabemampianina, D. Smith, J. M. Vidal, C. van de Vorstenbosch, A good practice guide to the administration of substances and removal of blood, including routes and volumes, Journal of applied toxicology: JAT 21(1) (2001) 15-23.
[55] P. V. Turner, T. Brabb, C. Pekow, M. A. Vasbinder, Administration of substances to laboratory animals: routes of administration and factors to consider, J Am Assoc Lab Anim Sci 50(5) (2011) 600-613.
[56] Q. Li, X. Li, E. Bury, K. Lackey, A. Koh, U. Wesselmann, T. Yaksh, C. Zhao, Hydration-Induced Void-Containing Hydrogels for Encapsulation and Sustained Release of Small Hydrophilic Molecules, Advanced Functional Materials n/a(n/a) 2301025.
[57] V.r. Kayser, G.l. Guilbaud, The analgesic effects of morphine, but not those of the enkephalinase inhibitor thiorphan, are enhanced in arthritic rats, Brain Res. 267(1) (1983) 131-138.
[58] V. Kayser, G. Guilbaud, Differential effects of various doses of morphine and naloxone on two nociceptive test thresholds in arthritic and normal rats, Pain 41(3) (1990) 353-363.
[59] D. Fletcher, V. Kayser, G. Guilbaud, Influence of timing of administration on the analgesic effect of bupivacaine infiltration in carrageenin-injected rats, Anesthesiology 84(5) (1996) 1129-1137.
[60] M. A. Khan, P. Gerner, G. K. Wang, Amitriptyline for prolonged cutaneous analgesia in the rat, Anesthesiology 96(1) (2002) 109-116.
[61] C. Stein, M. J. Millan, A. Herz, Unilateral inflammation of the hindpaw in rats as a model of prolonged noxious stimulation: alterations in behavior and nociceptive thresholds, Pharmacology, biochemistry, and behavior 31(2) (1988) 445-51.
[62] I. Decosterd, C. J. Woolf, Spared nerve injury: an animal model of persistent peripheral neuropathic pain, Pain 87(2) (2000) 149-158.
[63] K. Hargreaves, R. Dubner, F. Brown, C. Flores, J. Joris, A new and sensitive method for measuring thermal nociception in cutaneous hyperalgesia, Pain 32(1) (1988) 77-88.
[64] R. P. Bonin, C. Bories, Y. De Koninck, A simplified up-down method (SUDO) for measuring mechanical nociception in rodents using von Frey filaments, Molecular pain 10 (2014) 26.
[65] D. S. Brenner, J. P. Golden, R. W.t. Gereau, A novel behavioral assay for measuring cold sensation in mice, PloS one 7(6) (2012) e39765.
[66] Q. Liu, C. M. Santamaria, T. Wei, C. Zhao, T. Ji, T. Yang, A. Shomorony, B. Y. Wang, D. S. Kohane, Hollow Silica Nanoparticles Penetrate the Peripheral Nerve and Enhance the Nerve Blockade from Tetrodotoxin, Nano Letters 18(1) (2018) 32-37.
[67] A. Shomorony, C. M. Santamaria, C. Zhao, A. Y. Rwei, M. Mehta, D. Zurakowski, D. S. Kohane, Prolonged Duration Local Anesthesia by Combined Delivery of Capsaicin- and Tetrodotoxin-Loaded Liposomes, Anesthesia & Analgesia 129(3) (2019) 709-717.
[68] W. Zhang, T. Ji, Y. Li, Y. Zheng, M. Mehta, C. Zhao, A. Liu, D. S. Kohane, Light-triggered release of conventional local anesthetics from a macromolecular prodrug for on-demand local anesthesia, Nature Communications 11(1) (2020) 2323.
[69] C. Weldon, T. Ji, M.-T. Nguyen, A. Rwei, W. Wang, Y. Hao, C. Zhao, M. Mehta, B. Y. Wang, J. Tsui, R. P. Marini, D. S. Kohane, Nanoscale Bupivacaine Formulations To Enhance the Duration and Safety of Intravenous Regional Anesthesia, ACS Nano 13(1) (2019) 18-25.
[70] X. Li, Q. Li, S. Song, A. O. Stevens, Z. Broemmel, Y. He, U. Wesselmann, T. Yaksh, C. Zhao, Emulsion-Induced Polymersomes Taming Tetrodotoxin for Prolonged Duration Local Anesthesia, Advanced Therapeutics n/a(n/a) (2022) 2200199.
[71] T. Ji, Y. Li, X. Deng, A. Y. Rwei, A. Offen, S. Hall, W. Zhang, C. Zhao, M. Mehta, D. S. Kohane, Delivery of local anaesthetics by a self-assembled supramolecular system mimicking their interactions with a sodium channel, Nature Biomedical Engineering (2021).
[72] M. Mehta, C. Zhao, A. Liu, C. Innocent, D. S. Kohane, Prolonged Retrobulbar Local Anesthesia of the Cornea Does Not Cause Keratopathy in Mice, Translational Vision Science & Technology 11(1) (2022) 33-33.
[73] K. Sekimoto, M. Tobe, S. Saito, Local anesthetic toxicity: acute and chronic management, Acute medicine & surgery 4(2) (2017) 152-160.
[74] S. Yu, B. Wang, J. Zhang, K. Fang, The development of local anesthetics and their applications beyond anesthesia, 2019.
[75] Z. Lin, Y. Will, Evaluation of Drugs With Specific Organ Toxicities in Organ-Specific Cell Lines, Toxicological Sciences 126(1) (2011) 114-127.
[76] O. A. Almazroo, M. K. Miah, R. Venkataramanan, Drug Metabolism in the Liver, Clinics in liver disease 21(1) (2017) 1-20.
[77] K. E. Ibrahim, M. G. Al-Mutary, A. O. Bakhiet, H. A. Khan, Histopathology of the Liver, Kidney, and Spleen of Mice Exposed to Gold Nanoparticles, Molecules (Basel, Switzerland) 23(8) (2018).
[78] S. Sakamoto, W. Putalun, S. Vimolmangkang, W. Phoolcharoen, Y. Shoyama, H. Tanaka, S. Morimoto, Enzyme-linked immunosorbent assay for the quantitative/qualitative analysis of plant secondary metabolites, J Nat Med 72(1) (2018) 32-42.
[79] A. Y. Rwei, J.-J. Lee, C. Zhan, Q. Liu, M. T. Ok, S. A. Shankarappa, R. Langer, D. S. Kohane, Repeatable and adjustable on-demand sciatic nerve block with phototriggerable liposomes, Proceedings of the National Academy of Sciences 112(51) (2015) 15719-15724.
[80] D. S. Kohane, R. Langer, Biocompatibility and drug delivery systems, Chemical Science 1(4) (2010) 441-446.
[81] F. Yuan, N. Yosef, C. Lakshmana Reddy, A. Huang, S. C. Chiang, H. R. Tithi, E. E. Ubogu, CCR2 gene deletion and pharmacologic blockade ameliorate a severe murine experimental autoimmune neuritis model of Guillain-Barré syndrome, PloS one 9(3) (2014) e90463.
[82] C. Dong, S. P. Palladino, E. S. Helton, E. E. Ubogu, The pathogenic relevance of aM-integrin in Guillain-Barré syndrome, Acta Neuropathologica 132(5) (2016) 739-752.
[83] N. Percie du Sert, V. Hurst, A. Ahluwalia, S. Alam, M. T. Avey, M. Baker, W. J. Browne, A. Clark, I. C. Cuthill, U. Dirnagl, M. Emerson, P. Garner, S. T. Holgate, D. W. Howells, N. A. Karp, S. E. Lazic, K. Lidster, C. J. MacCallum, M. Macleod, E. J. Pearl, O. H. Petersen, F. Rawle, P. Reynolds, K. Rooney, E. S. Sena, S. D. Silberberg, T. Steckler, H. WUrbel, The ARRIVE guidelines 2.0: Updated guidelines for reporting animal research, BMC Veterinary Research 16(1) (2020) 242.
[84] S. V. Frye, M. R. Arkin, C. H. Arrowsmith, P. J. Conn, M. A. Glicksman, E. A. Hull-Ryde, B. S. Slusher, Tackling reproducibility in academic preclinical drug discovery, Nature reviews. Drug discovery 14(11) (2015) 733-4.
[85] S. P. Jones, X.-L. Tang, Y. Guo, C. Steenbergen, D. J. Lefer, R. C. Kukreja, M. Kong, Q. Li, S. Bhushan, X. Zhu, J. Du, Y. Nong, H. L. Stowers, K. Kondo, G. N. Hunt, T. T. Goodchild, A. Orr, C. C. Chang, R. Ockaili, F. N. Salloum, R. Bolli, The NHLBI-Sponsored Consortium for preclinicAl assESsment of cARdioprotective Therapies (CAESAR), Circulation Research 116(4) (2015) 572-586.
[86] S. Perrin, Preclinical research: Make mouse studies work, Nature 507(7493) (2014) 423-425.
[87] Q. Li, X. Li, C. Zhao, Strategies to Obtain Encapsulation and Controlled Release of Small Hydrophilic Molecules, Front Bioeng Biotechnol 8 (2020) 437-437.
[88] Y. Sun, M. Zabihi, Q. Li, X. Li, B. J. Kim, E. E. Ubogu, S. N. Raja, U. Wesselmann, C. Zhao, Drug Permeability: From the Blood-Brain Barrier to the Peripheral Nerve Barriers, Advanced Therapeutics n/a(n/a) 2200150.
[89] K. Ren, R. Dubner, Inflammatory Models of Pain and Hyperalgesia, ILAR Journal 40(3) (1999) 111-118.

1. A compound of Formula I:
Z-L-X (I)
or pharmaceutically acceptable salt thereof, wherein:
Z is selected from the group consisting of capsaicin, bupivacaine, tetracaine, lidocaine, benzocaine, procaine, prilocaine, cinchocaine, and ropivacaine;
L is an optional divalent linker; and
X is a monosaccharide;
wherein when L is present, L is covalently bonded to X by —O—, and L is covalently bonded to Z through —O— or —N—.

2. (canceled)

3. (canceled)

4. The compound of claim 1, wherein X is selected from the group consisting of glucose, galactose, mannose, and glucosamine.

5. The compound of claim 1, wherein X is glucose.

6. (canceled)

7. The compound of claim 1, wherein L is present and is a hydrolysable divalent linker.

8. The compound of claim 1, wherein L is present and is a linker selected from the group consisting of —SO₂, —SO₂R′; SO₂R′R″, —SO₂NR′R″; —SO₂NR′R″C(═O); —NR′SO₂R″; —R′SO₂NR′R′″; —C(═O); —C(═O)R′; —OC(═O)R′; —OC(═O)R″″C(═O)O—; —C(═O)R′C(═O)—; —C(═O)NR′R″; —NR′C(═O)R″; —NR′C(═O)R″″C(═O); —OR′; —NR′R″; -SR′; —N₃—C(═O)OR′; —O(CR′R″)ᵣC(═O)R′; —O(CR′R″)ᵣNR″C(═O)R′; —O(CR′R″)ᵣNR″SO₂R′; —OC(═O)NR′R″; —NR′C(═O)OR″; and substituted or unsubstituted C₁-C₆ aliphatic alkyl; wherein R′, R″, and R′″ are individually selected from hydrogen; substituted or unsubstituted alkyl; substituted or unsubstituted alkenyl; substituted or unsubstituted ether; substituted or unsubstituted cycloalkyl; substituted or unsubstituted heterocyclyl; substituted or unsubstituted cycloalkenyl, substituted or unsubstituted aryl, substituted or unsubstituted heteroaryl, substituted or unsubstituted arylalkyl, substituted or unsubstituted heteroalkyl, substituted or unsubstituted alkylheteroaryl, or substituted or unsubstituted amine; R″″ is selected from substituted or unsubstituted alkyl; substituted or unsubstituted alkenyl; substituted or unsubstituted ether; substituted or unsubstituted cycloalkyl; substituted or unsubstituted heterocyclyl; substituted or unsubstituted cycloalkenyl, substituted or unsubstituted aryl, substituted or unsubstituted heteroaryl, substituted or unsubstituted arylalkyl, substituted or unsubstituted heteroalkyl, substituted or unsubstituted alkylheteroaryl, or substituted or unsubstituted amine; and r is an integer from 1 to 6.

9. The compound of claim 8, wherein L is present and is —O—C(═O)—.

10. The compound of claim 8, wherein L is present and is —C(═O)R′C(═O)—, and R′ is C₁₋₄alkyl.

11. (canceled)

12. (canceled)

13. The compound of claim 1, wherein Z is capsaicin.

14. The compound of claim 1, wherein Z is bupivacaine.

15. The compound of claim 1, wherein Z has the formula:

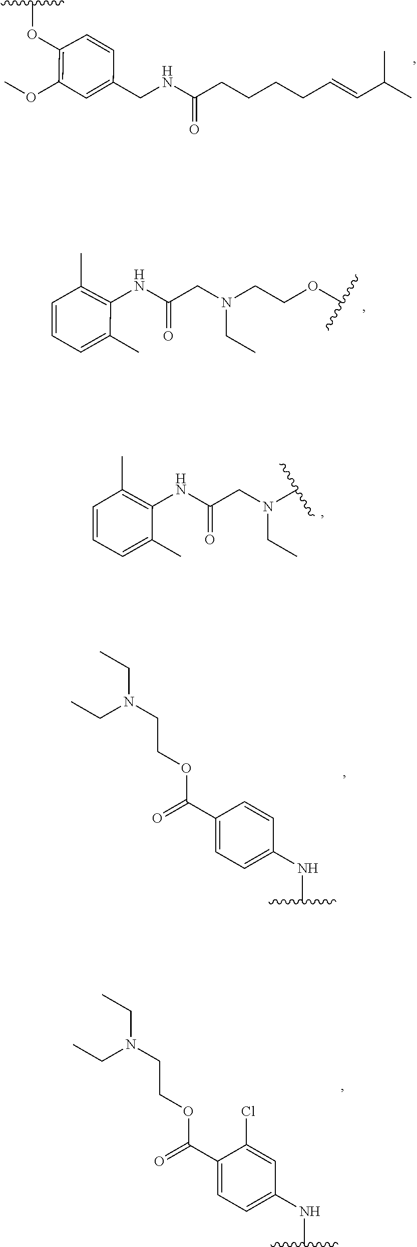

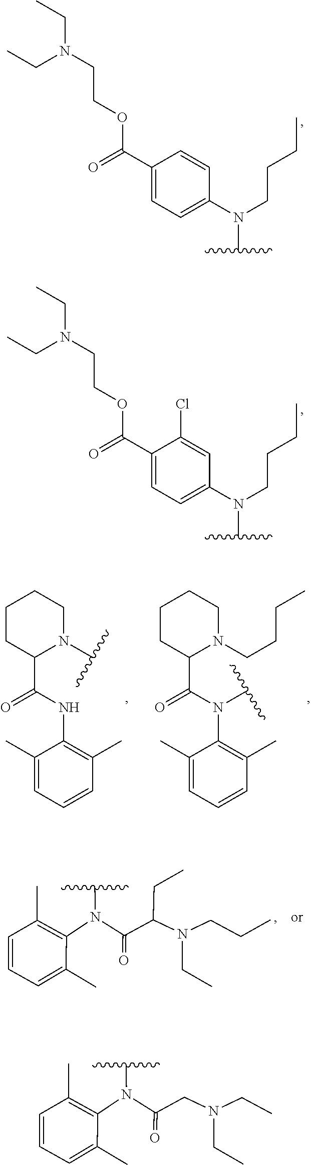

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. The compound of claim 1, wherein the compound is selected from: 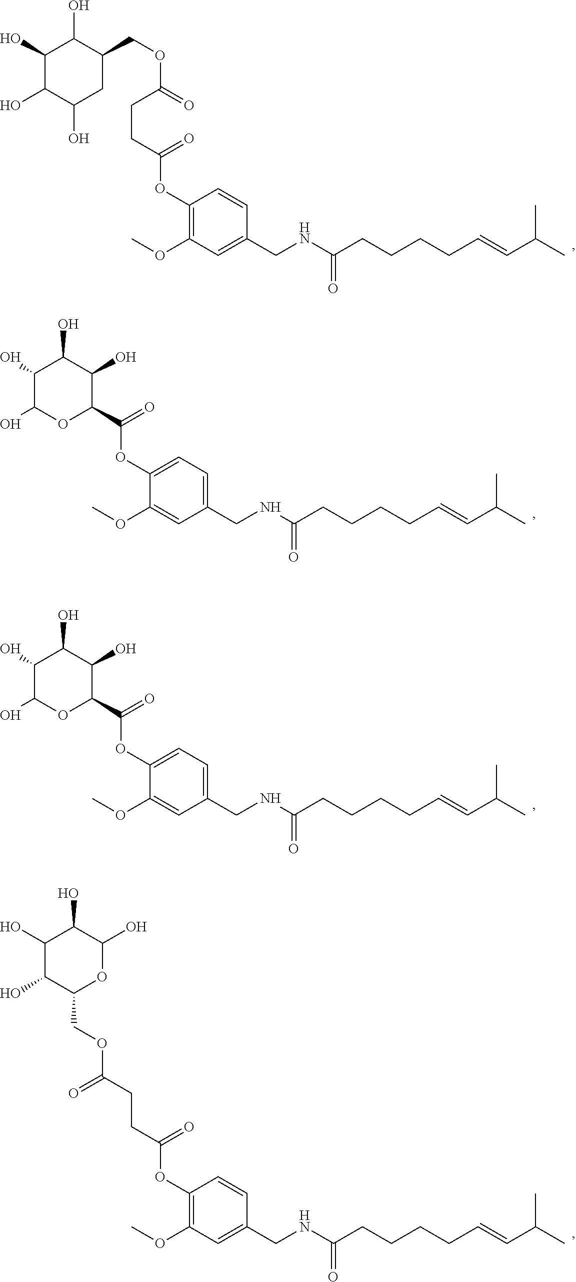 [SMILES: COc1cc(CNC(=O)CCCC/C=C/C(C)C)ccc1OC(=O)CCC(=O)OC[C@H]1CC(O)C(O)[C@@H](O)C1O ; COc1cc(CNC(=O)CCCC/C=C/C(C)C)ccc1OC(=O)[C@H]1OC(O)[C@H](O)[C@@H](O)[C@H]1O ; COc1cc(CNC(=O)CCCC/C=C/C(C)C)ccc1OC(=O)[C@H]1OC(O)[C@H](O)[C@@H](O)[C@H]1O ; COc1cc(CNC(=O)CCCC/C=C/C(C)C)ccc1OC(=O)CCC(=O)OC[C@H]1OC(O)[C@H](O)C(O)[C@H]1O] 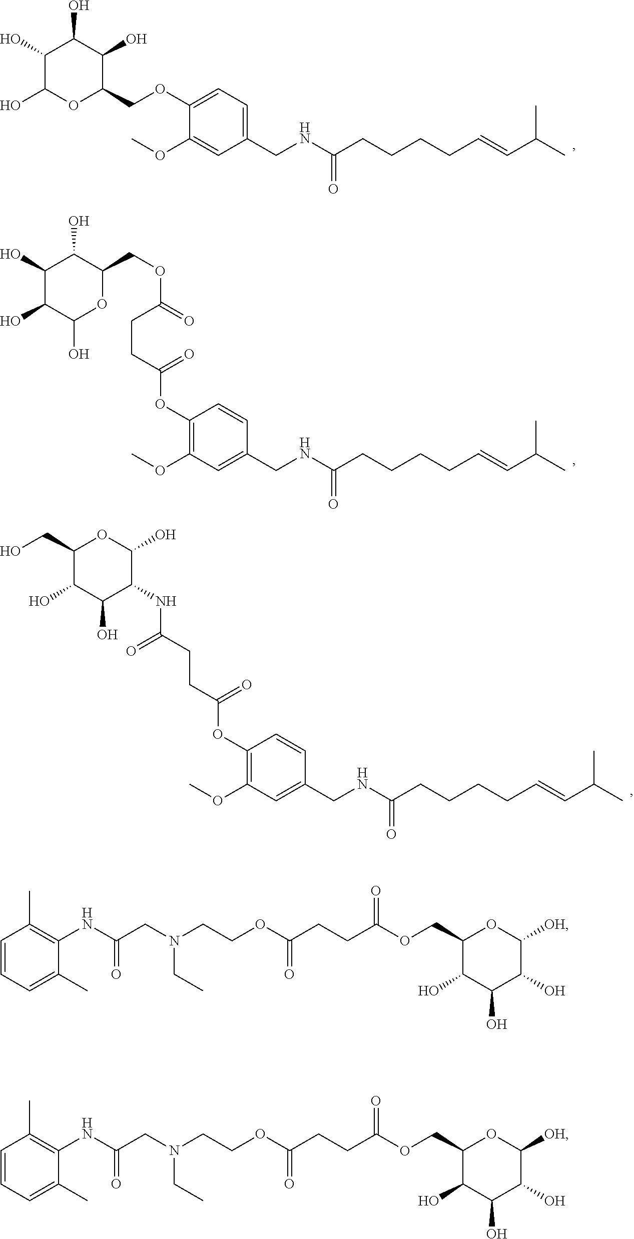 [SMILES: COc1cc(CNC(=O)CCCC/C=C/C(C)C)ccc1OC[C@H]1OC(O)[C@H](O)[C@@H](O)[C@H]1O ; COc1cc(CNC(=O)CCCC/C=C/C(C)C)ccc1OC(=O)CCC(=O)OC[C@H]1OC(O)[C@@H](O)[C@@H](O)[C@@H]1O ; COc1cc(CNC(=O)CCCC/C=C/C(C)C)ccc1OC(=O)CCC(=O)N[C@@H]1[C@@H](O)[C@H](O)[C@@H](CO)O[C@@H]1O ; CCN(CCOC(=O)CCC(=O)OC[C@H]1O[C@H](O)[C@H](O)[C@@H](O)[C@@H]1O)CC(=O)Nc1c(C)cccc1C ; CCN(CCOC(=O)CCC(=O)OC[C@H]1O[C@@H](O)[C@H](O)[C@@H](O)[C@H]1O)CC(=O)Nc1c(C)cccc1C] 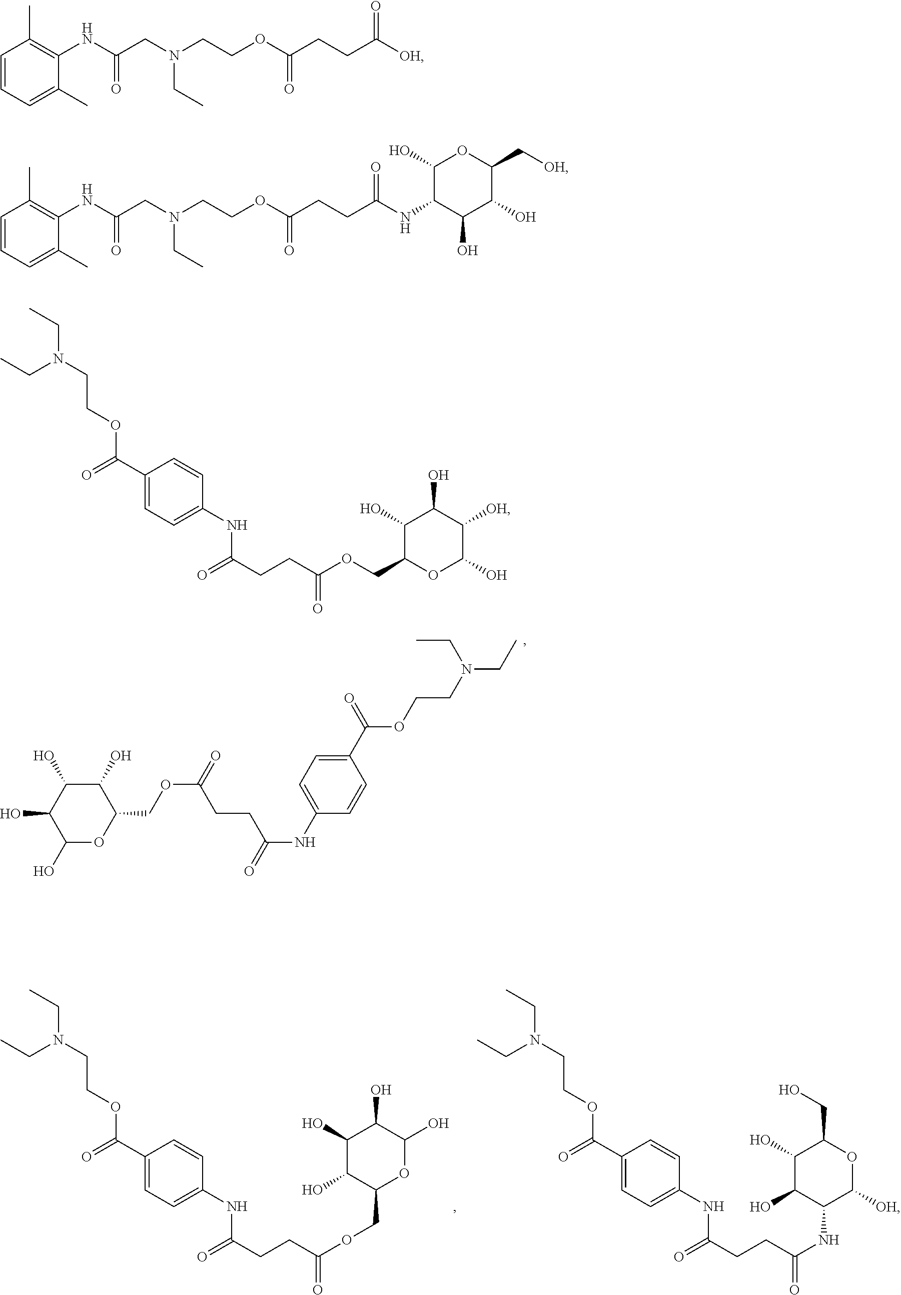 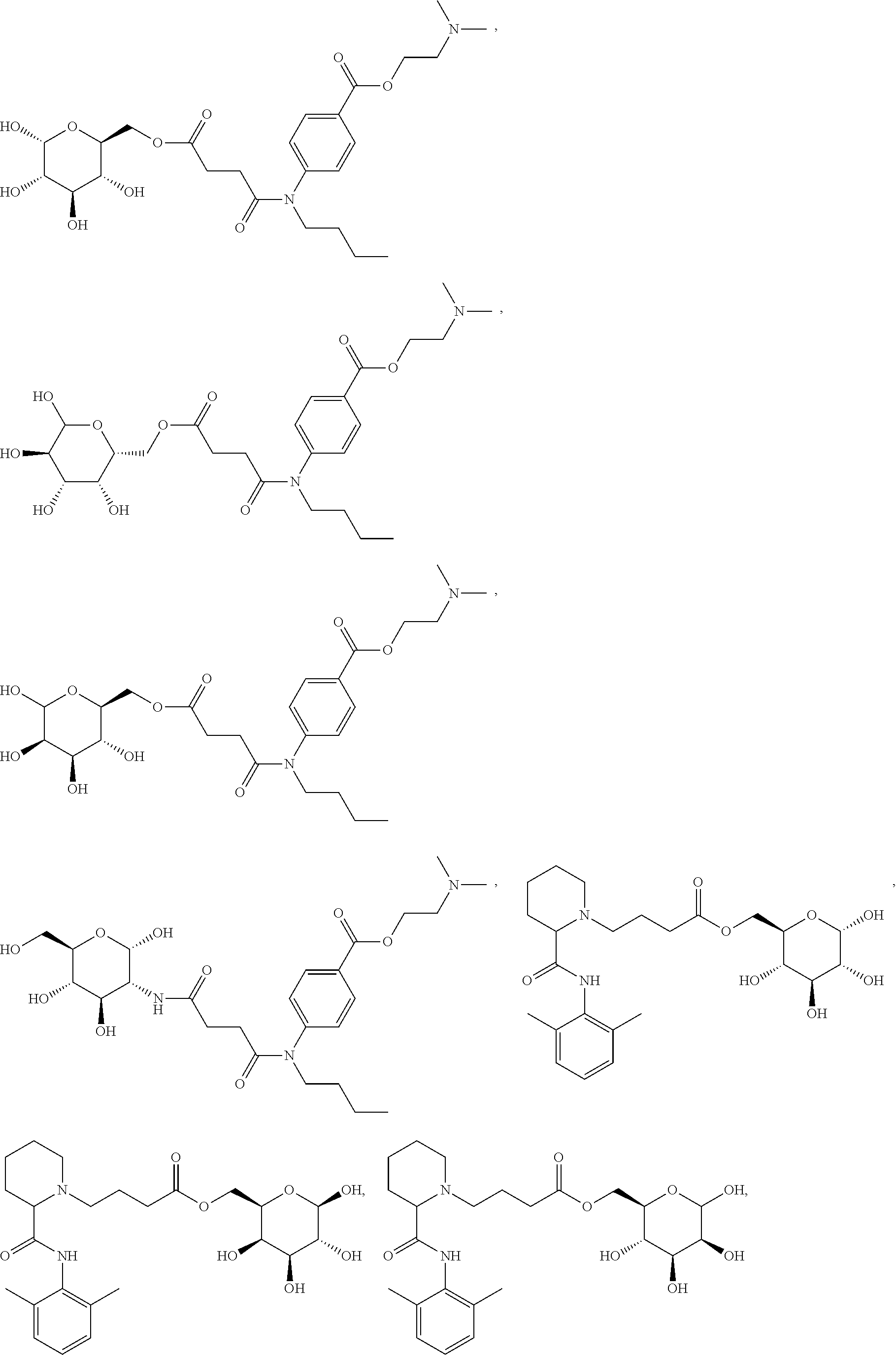 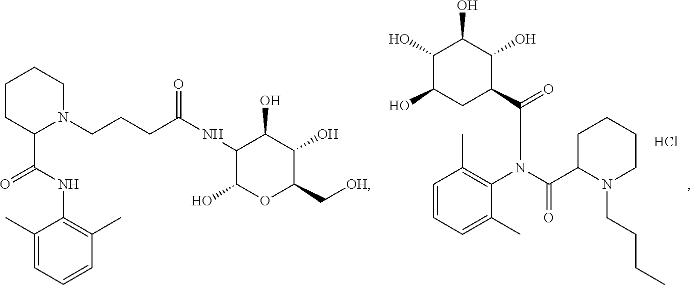 [SMILES: Cc1cccc(C)c1NC(=O)C1CCCCN1CCCC(=O)NC1[C@@H](O)[C@H](O)[C@@H](CO)O[C@@H]1O ; CCCCN1CCCCC1C(=O)N(C(=O)[C@H]1C[C@@H](O)[C@H](O)[C@@H](O)[C@@H]1O)c1c(C)cccc1C ; Cl ; ","]
or pharmaceutically acceptable salts thereof.

28. The compound of claim 27, wherein the compound is 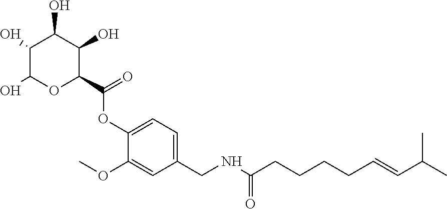 [SMILES: COc1cc(CNC(=O)CCCC/C=C/C(C)C)ccc1OC(=O)[C@H]1OC(O)[C@H](O)[C@@H](O)[C@H]1O]
or a pharmaceutically acceptable salt thereof.

29. The compound of claim 27, wherein the compound is 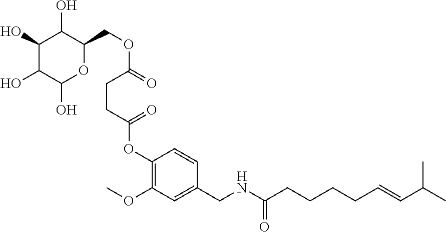 [SMILES: COc1cc(CNC(=O)CCCC/C=C/C(C)C)ccc1OC(=O)CCC(=O)OC[C@H]1OC(O)C(O)[C@@H](O)C1O]
or a pharmaceutically acceptable salt thereof.

30. (canceled)

31. (canceled)

32. (canceled)

33. (canceled)

34. A method of treating and preventing pain in a subject in need thereof by using a compound of claim 1.

35. (canceled)

36. (canceled)

37. A compound of Formula I:
Z-L-X (I)
or pharmaceutically acceptable salt thereof, wherein:
Z is selected from the group consisting of capsaicin, bupivacaine, tetracaine, lidocaine, benzocaine, procaine, prilocaine, cinchocaine, and ropivacaine;
L is —C(═O)R′C(═O)—, and R′ is C₁₋₄alkyl;
X is H;
L is covalently bonded to X by —O—; and
L is covalently bonded to Z through —O— or —N—.

38. The compound of claim 37, wherein Z is capsaicin.

39. The compound of claim 37, wherein Z is bupivacaine.

40. The compound of claim 37, wherein Z has the formula:

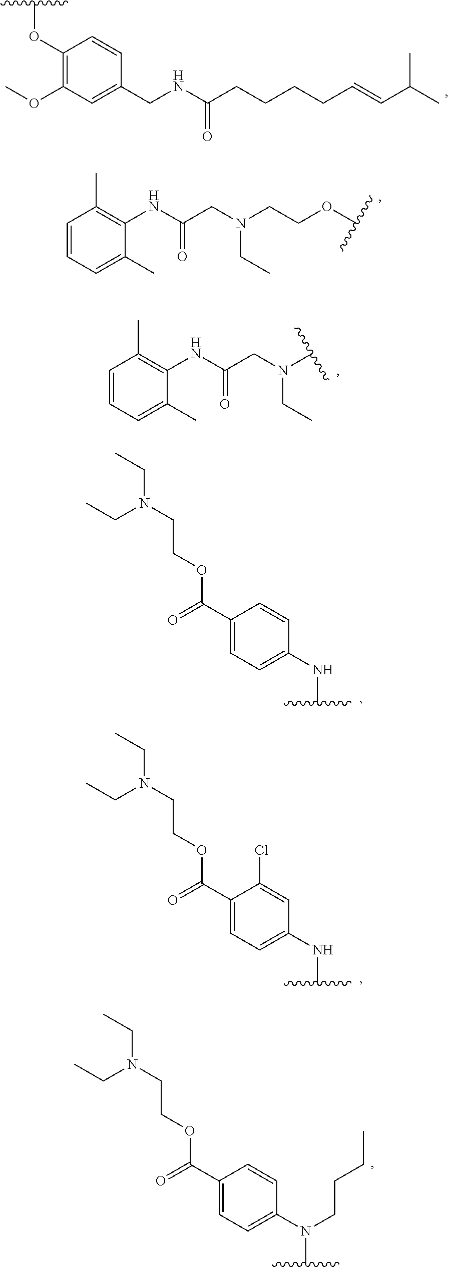

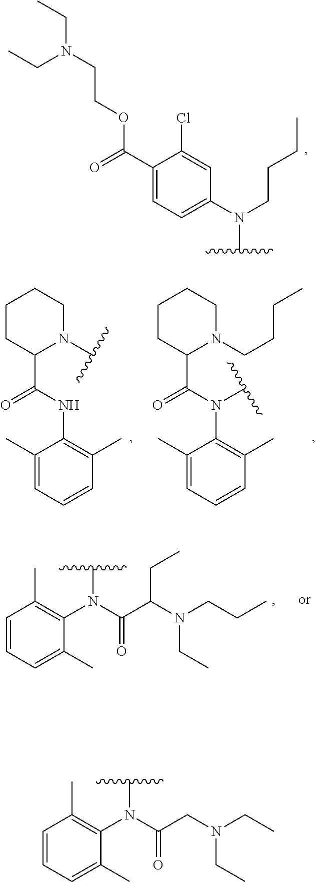

41. The compound of claim 37, wherein -L-X is —C(═O)[CH₂]ₙCOOH or —C(═O)[CH₂]ₙCONH₂, wherein n is 1-6.

42. The compound of claim 37, wherein the compound is selected from: 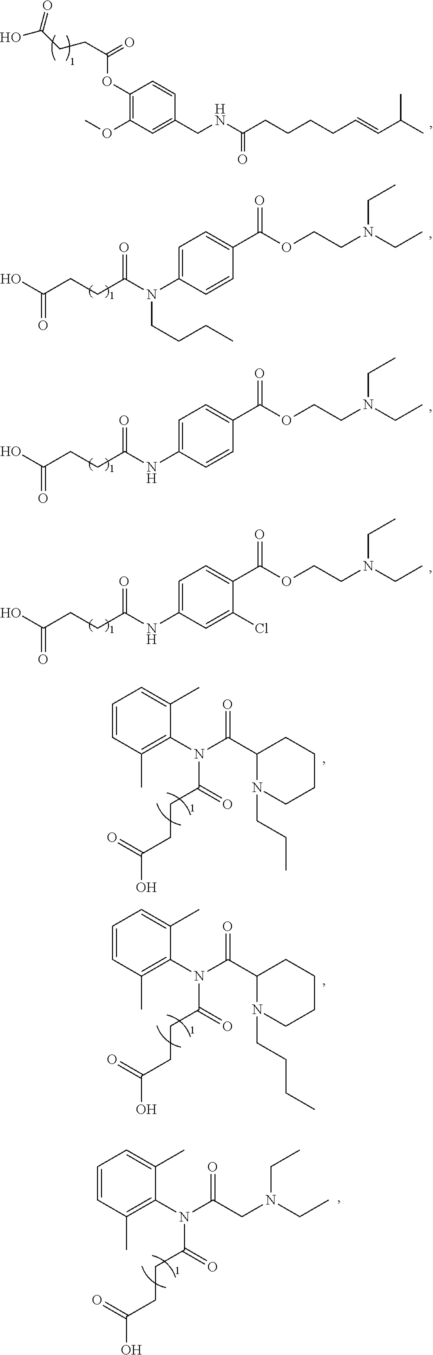 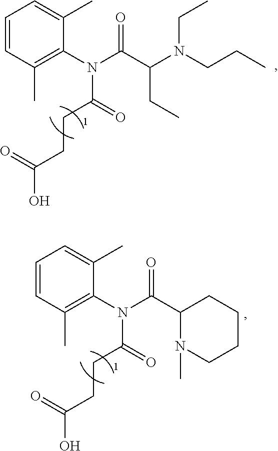
or pharmaceutically acceptable salts thereof.
